(19) United States
(12) Patent Application Publication
(10) Pub. No.: US 2025/0101062 A1
(43) Pub. Date: Mar. 27, 2025

(54) ANTIMICROBIAL PEPTIDES

(71) Applicant: Board of Regents of the University of Nebraska, Lincoln, NE (US)

(72) Inventors: Guangshun Wang, Omaha, NE (US); Abraham Mechesso, Omaha, NE (US)

(21) Appl. No.: 18/897,413

(22) Filed: Sep. 26, 2024

Related U.S. Application Data
(60) Provisional application No. 63/540,493, filed on Sep. 26, 2023.

(51) Int. Cl. C07K 7/06; A61K 38/00; A61P 31/04; C12Q 1/18

(52) U.S. Cl. CPC C07K 7/06; A61P 31/04; C12Q 1/18; A61K 38/00

(57) ABSTRACT

Antimicrobial peptides and methods of use are provided.

This application claims priority under 35 U.S.C. § 119(e) to U.S. Provisional Patent Application No. 63/540,493, filed on Sep. 26, 2023. The foregoing application is incorporated by reference herein.

This invention was made with government support under Grant Nos. R01 GM138552 and R56 AI175209 awarded by the National Institutes of Health. The government has certain rights in the invention.

Incorporated herein by reference in its entirety is the Sequence Listing submitted on Sep. 26, 2024 as a XML file named SeqList, created Sep. 25, 2024, and having a size of 51,694 bytes.

### FIELD OF THE INVENTION

The present invention relates to the field of antimicrobial peptides and the treatment of microbial infections. More specifically the invention provides anti-microbial peptides and methods of using such peptides for the inhibition, treatment, and/or prevention of microbial infections.

### BACKGROUND OF THE INVENTION

Identification of novel antibiotics is of top importance because of the threat of antibiotic-resistant pathogens. A British report projected the deaths of 10 million people by 2050 (O'Neill, J. (2014) Review on Antimicrobial Resistance: Tackling Drug-Resistant Infections Globally; CABI). Because of lasting potency, antimicrobial peptides (AMPs) are important candidates for developing future antibiotics (Harwig, et al. (1992) Blood 79:1532-1537; Overhage, et al. (2008) Infect. Immun., 76:4176-4182; Diamond, et al. (1993) Proc. Natl. Acad. Sci., 90:4596-4600; Gudmundsson, et al. (1996) Eur. J. Biochem., 238:325-332; Wang, G. (2014) Pharmaceuticals 7:545-594). According to the AMP database (aps.unmc.edu), over 3000 AMPs have been isolated and characterized from six life kingdoms, including bacteria, archaea, protists, fungi, plants, and animals (Wang, et al. (2016) Nucleic Acids Res., 44:D1087-D1093; Wang, et al. (2022) Methods Enzymol., 663:1-18). Like plants and insects, humans also deploy multiple AMPs in response to a variety of invading pathogens, including bacteria, fungi, viruses, and parasites. While there are numerous defensins in humans (Harwig, et al. (1992) Blood 79:1532-1537; Jia, et al. (1999) Infect. Immun., 67:4827-4833), a single cathelicidin gene is mapped in the human genome (Gudmundsson, et al. (1996) Eur. J. Biochem., 238:325-332; Johansson, J.; Gudmundsson, et al. (1998) J. Biol. Chem., 273:3718-3724). Different from human α- and β-defensins, which comprise three pairs of disulfide bonds, human cathelicidin LL-37 does not contain cysteine and belongs to the linear peptide class. LL-37 is the most widely investigated form of human cathelicidin peptides with different lengths.

Earlier studies learned that the structure and activity of LL-37 depend on environmental conditions (Johansson, et al. (1998) J. Biol. Chem., 273:3718-3724). In standard Mueller-Hinton Broth (MHB) medium, antibacterial activity for LL-37 was observed against Gram-negative pathogens but not Gram-positive pathogens, such as Staphylococcus aureus (Turner, et al. (1998) Antimicrob. Agents Chemother., 42:2206-2214). It was hypothesized that acidic components in the medium were responsible for masking the peptide activity. Hence, an easily accessible and cost-effective medium is required for the antimicrobial susceptibility assays of LL-37 and other antimicrobial agents.

Antimicrobial peptides activities are often compromised in the presence of physiological salts, pH, and serum. Furthermore, longer peptide sequences are cost prohibitive to manufacture and invite the development of antimicrobial resistance. Accordingly, there is a strong need for improved, short antimicrobial peptides.

### SUMMARY OF THE INVENTION

In accordance with one aspect of the instant invention, peptides, particularly antimicrobial peptides, are provided. In certain embodiments, the peptides comprise at least one D-amino acid. In certain embodiments, the peptides are amidated. In certain embodiments, the peptides have 12 or fewer amino acids.

Compositions comprising at least one peptide of the instant invention and at least one pharmaceutically acceptable carrier are also provided. The compositions may further comprise at least one other antimicrobial compound (e.g., antibiotic).

In accordance with another aspect of the instant invention, methods for inhibiting, treating, and/or preventing a microbial infection or biofilm are provided. The methods can be performed in vitro or in vivo. Generally, the methods comprise contacting the microbes with a peptide of the instant invention. In certain embodiments, the methods comprise administering (e.g., topically) to a subject at least one antimicrobial peptide of the instant invention, particularly as a composition with a carrier. In certain embodiments, the methods further comprise the administration at least one other antimicrobial treatment, such as the administration of at least one additional antibiotic.

In accordance with another aspect of the instant invention, methods of screening a compound for antibacterial activity are provided. The methods comprise contacting bacteria with a test compound in a diluted media and measuring the cytotoxicity of the compound. In certain embodiments, the diluted media is a diluted Mueller-Hinton Broth (MHB).

In accordance with another aspect of the instant invention, methods for generating a peptide with antimicrobial activity against bacteria, particularly Gram-negative bacteria, are provided. In certain embodiments, the method comprises designing peptides which satisfy the following criteria: a) the peptide is 8 to 12 amino acids in length; b) at least 50% of the N-terminal four amino acids of the peptide are R or K; c) the hydrophobicity of the peptide is at least 40%; and d) the peptide has a net charge of at least +3.

### BRIEF DESCRIPTIONS OF THE DRAWING

FIG. 1 provides a sequence relationship of human cathelicidin LL-37 (LLGDFFRKSKEKIGKEFKRIVQRIKDFLRNLVPRTES; SEQ ID NO: 1) with its antimicrobial fragments LL-31 (LLGDFFRKSKEKIGKEFKRIVQRIKDFLRNL; SEQ ID NO: 2), SK-24 (SKEKIGKEFKRIVQRIKDFLRNLV; SEQ ID NO: 3), FK-16 (FKRIVQRIKDFLRNLV; SEQ ID NO: 4), KR-12 (KRIVQRIKDFLR; SEQ ID NO: 5), and KR-8 (KRIVQRIK; SEQ ID NO: 6). The 3D structure of LL-37 (PDB: 2K6O) bound to membrane-mimetic lipids was determined by 3D triple-resonance NMR spectroscopy (Wang, G. (2008) J. Biol. Chem., 283:32637-32643). KR-8, the template for designing LL-37mini, is the smallest active peptide from LL-37 newly discovered in a sensitive medium.

FIGS. 2A-2D show biofilm disruption activity of human LL-37 (FIGS. 2A and 2B) and 17BIPHE2 (FIGS. 2C and 2D) against the 24 hour preformed biofilms of S. aureus USA300 LAC in different MHB media: 12.5% MHB (FIGS. 2A and 2C) and 100% MHB (FIGS. 2B and 2D). FIGS. 2E-2H show the treatment outcome of LL-37 (FIGS. 2E and 2F) and 17BIPHE2 (FIGS. 2G and 2H) against the 24-hour preformed biofilms of E. coli E423-17 in different MHB media: 12.5% MHB (FIGS. 2E and 2G) and 100% MHB (FIGS. 2F and 2H). FIG. 2I shows the effects of medium dilution on antibacterial activity (MIC in μM) of a panel of antibiotics. Except for nafcillin and amikacin with small changes in the averaged MIC value, essentially no changes in antibacterial activity was observed with the dilution of MHB from 100% to 12.5%. These results underscore the usefulness of the diluted MHB for antimicrobial screening. Similar screening results for LL-37 peptides in DMEM are shown in Table 5. Growth curves of S. aureus USA 300 LAC (FIGS. 2J-2K) and P. aeruginosa 152 (FIGS. 2L-2M) in 12.5%, 25%, 50% and 100% MHB or TSB medium (curves from bottom to top). These figures indicate that media dilution had a relatively small effect on the growth of S. aureus (FIGS. 2J-2K) but a more pronounced effect on the P. aeruginosa strain isolated from wound (FIGS. 2L-2M).

FIG. 3 shows antimicrobial screening of ultrashort LL-37 fragments in 12.5% MHB leading to the identification of KR-8 and RIK-10 (peptide activity in micromolar). EC12.5% means the anti-E. coli assay in 12.5% NMB, while SA12.5% stands for the anti-S. aureus assay in 12.5% NMB. Likewise, 100% means rich NMB without dilution. Amino acid sequences are aligned. LL-37 (SEQ ID NO: 1), KR-12 (SEQ ID NO: 5), LL-10 (SEQ ID NO: 7), KE-10 (SEQ ID NO: 8), LR-10 (SEQ ID NO: 9), RK-9 (SEQ ID NO: 10), RIK-10 (SEQ ID NO: 11), KR-8 (SEQ ID NO: 6), and FK-16 (SEQ ID NO: 4) are provided.

FIGS. 4A and 4B show the cytotoxicity of a series of peptides W538-W544 designed based on KR-8 (i.e., W538) by hydrophobic substitution. FIG. 4A shows the hemolysis of human red blood cells due to the treatment of W538-W544 at various peptide concentrations. FIG. 4B shows the viability of human skin HaCaT cells due to the treatment of W538-W544 at various peptide concentrations. Daptomycin was included as a control. The results were expressed as mean±standard deviation.

FIGS. 5A-5F show the killing kinetics and mechanism of action of LL-37 mini and bacterial resistance development. FIG. 5A: LL-37 mini (2×MIC, 16 μM) killed the exponential phase of S. aureus USA300 LAC in 120 minutes, similar to daptomycin. FIGS. 5B and 5C provide the permeabilization (FIG. 5B) and depolarization (FIG. 5C) of the membranes of S. aureus USA300 LAC by LL-37 mini treated at 1×MIC of W543 (8 μM), daptomycin (1 μM), and gentamicin (1 μM). RFU: relative fluorescence intensity. FIGS. 5D and 5E provide SEM images of untreated (FIG. 5D) and LL-37 mini-treated (FIG. 5E) S. aureus. FIG. 5F shows the resistance development of S. aureus USA300 LAC in a multiple passage experiment in the presence of sub-MIC levels of LL-37 mini (rectangle), nafcillin (diamond), and daptomycin (triangle). Results were expressed as mean±standard deviation. Resistance development is indicated by a substantial increase in MIC. FIGS. 5G and 5H show membrane permeabilization (FIG. 5G) and depolarization (FIG. 5H) of E. coli E423-17 by LL-37mini at 1×MIC of W543 (8 μM), colistin (2 μM), and gentamicin (1 μM). RFU: Relative fluorescence intensity.

FIGS. 6A-6F show the antibiofilm activity of LL-37mini against S. aureus USA300 in vitro and in vivo. Bacterial attachment (FIG. 6A), biofilm inhibition (FIG. 6B), and disruption of biofilms formed for 24 hours (FIG. 6C), 48 hours (FIG. 6D), and 72 hours (FIG. 6E) in 100% TSB media are provided. FIG. 6F shows antibiofilm efficacy of LL-37 in murine wounds. S. aureus USA300 biofilms were formed in the wounds created on the back of BALB/c mice for 24 hours, followed by treatment with water (untreated control), LL-37mini (4.4 mg/kg per wound), or daptomycin (10 mg/kg per wound) for 24 hours. Results were expressed as mean±standard deviation. FIG. 6G shows LL-37mini can also disrupt the 24 hour preformed biofilms of E. coli E423-17. Live bacteria left in the treated biofilms were determined by the XTT method. FIGS. 6H and 6I show antimicrobial activity (MIC, μM) of short LL-37 fragments in 12.5%, 25%, 50% and 100% NMB medium against S. aureus USA300 LAC (FIG. 6H) and S. aureus USA300 mprF disrupted transposon strain (FIG. 6I).

FIGS. 7A-7D show a peptide stability evaluation with and without proteases. In this assay, growth curves of S. aureus USA300 LAC were plotted with and without protease pretreatment of L- and D-forms of LL-37mini: without protease (no growth at 8 μM) (FIG. 7A), trypsin (FIG. 7B), chymotrypsin (FIG. 7C), and pepsin (FIG. 7D) at a peptide:protease molar ratio of 40:1. Peptides were incubated with proteases at 37° C. for 1 hour before mixing with the bacterial culture. Results were expressed as mean±standard deviation. This experiment provides for stability screening of peptides (Wang, et al. (2014) ACS Chem. Biol., 9:1997-2002).

FIGS. 8A-8C provide the LL-37 structure, active region discovery and peptide design strategies. FIG. 8A provides in membrane-bound state, human cathelicidin LL-37 adopts a helical structure spanning residues 1-31, while the C-terminal residues 32-37 are not structured (PDB ID: 2K6O) (Wang, G. (2008) J. Biol. Chem., 283(47):32637-32643). Identification of the antibacterial regions of LL-37 via structural studies and library approach (FIG. 8B) and direct use of short segments of LL-37 for peptide design (FIG. 8C). FIG. 8B provides LL-37 (SEQ ID NO: 1), LL-31 (SEQ ID NO: 2), SK-24 (SEQ ID NO: 3), FK-16 (SEQ ID NO: 4), KR-12 (SEQ ID NO: 5), and RIK-10 (SEQ ID NO: 11). FIG. 8C provides LL-37 (SEQ ID NO: 1), LL-10 (SEQ ID NO: 7), RK-9 (SEQ ID NO: 10), KR-8 (SEQ ID NO: 6), and RIK-10 (SEQ ID NO: 11).

FIG. 9 provides helical wheel plots for the peptides designed based on short segments of LL-37. Helical wheel plots of LL-10+, RK-9+, and RIK-10+ are presented at the top. The helical wheel plots for reversed sequences of LL-10+, RK-9+, and RIK-10+ are presented at the bottom.

FIGS. 10A-10H provide growth curves of S. aureus USA 300 (FIGS. 10A-10D) and P. aeruginosa E411-17 (FIGS. 10E-10H) treated with LL-10+, RK-9+, KR-8+, and RIK-10+ in MHB. Curves are 32 μM, 16 μM, 8 μM, 4 μM, 2 μM, or untreated from bottom to top. FIGS. 10I and 10J provide killing kinetics of RIK-10+ against S. aureus USA300 (FIG. 10I) and P. aeruginosa E411-17 (FIG. 10J). RIK-10+ (4×MIC) killed the exponential phase of S. aureus USA300 LAC and P. aeruginosa E411-17 in 90 and 60 minutes, respectively. Results were expressed as mean±standard deviation.

FIGS. 11A-11D show peptide dose-dependent cytotoxicity to human red blood cells (RBC) (FIG. 11A), human skin HaCaT cells (FIG. 11B), human HepG2 liver cells (FIG. 11C), and human A549 lung cells (FIG. 11D). Colistin and daptomycin were included as controls. The results were expressed as mean standard deviation.

FIGS. 12A-12F show designer peptides permeabilized the membranes of S. aureus USA300 (FIGS. 12A-12C) and P. aeruginosa E411-17 (FIGS. 12D-12F) as indicated by a dose-dependent fluorescence increase. RFU: Relative fluorescence intensity. Results were expressed as mean±standard deviation. FIGS. 12G and 12H provide membrane permeation of E. coli (FIG. 12G) and A. baumannii (FIG. 12H) treated with the four designed peptides LL-10+, RK-9+, KR-8+, and RIK-10+ and their parent peptide human LL-37. FIGS. 12I-12N show the designed peptides depolarized the membranes of S. aureus USA300 (FIGS. 12I-12K) and P. aeruginosa E411-17 (FIGS. 12L-12N). RFU: Relative fluorescence intensity. Results were expressed as mean±standard deviation.

FIGS. 13A-13D show the inhibition of bacterial biofilm formation and disruption of preformed biofilms in vitro. FIGS. 13A-13B show the surface attachment of S. aureus USA300 (FIG. 13A) and P. aeruginosa E411-17 (FIG. 13B). FIGS. 13C-13D show the metabolic activity of the 24-hour preformed biofilms of S. aureus USA300 (FIG. 13C) and P. aeruginosa E411-17 (FIG. 13D) after peptide treatment for 24 hours. The metabolic activity of the biofilm-associated bacteria was assessed using the formazan-based MTT assay on biofilms formed for 24 hours in TSB media.

FIG. 14 shows the anti-biofilm activity of the four designer peptides against S. aureus USA300 in vivo. S. aureus USA300 biofilms were formed in the wounds created on the back of BALB/c mice for 24 hours followed by treatment with water (untreated control), KR-8+ (7.7 mg/kg per wound), RK-9+ (7.6 mg/kg per wound), LL-10+ (9.4 mg/kg per wound), RIK-10+ (9 mg/kg per wound) or daptomycin (10.5 mg/kg per wound) for 24 hours. Results were expressed as mean±standard deviation.

FIGS. 15A-15D provide three-dimensional structures of RIK-10+, a peptide designed based on RIK-10 derived from the major antimicrobial region of human cathelicidin LL-37. The NMR structure was determined in a membrane-mimetic environment (peptide:DPC molar ratio: 1:60) at 25° C. and pH 4.5. FIG. 15A provides the peptide backbone; FIG. 15B provides a ribbon structure; FIG. 15C provides a view of the side chains from one end; and FIG. 15D provides the surface characteristics.

FIG. 16 provides charge density plots of LL-10+, RK-9+, KR-8+, and RIK-10+ and their reversed sequences. Amino acids are provided on x-axis.

FIG. 17 provides hydrophobic density plots of LL-10+. RK-9+, KR-8+, and RIK-10+ and their reversed sequences. Amino acids are provided on x-axis.

FIGS. 18A and 18B provide the charge (FIG. 18A) and hydrophobic (FIG. 18B) density plots of human LL-37.

FIGS. 19A-19L provide charge density plots of selected helical antimicrobial peptides with activity against Gram-negative bacteria. These peptides contain less than 50 amino acids and contain both arginine and lysine. These filtering criteria led to the identification of 11 natural AMPs (FIGS. 19B-19L), which are listed in Table 13. For comparison, the charge-density plot of part of LL-37 (AP00310) is provided in FIG. 19A (SEQ ID NO: 18). FIGS. 19B-19L provide SEQ ID NOs: 19-29, respectively.

### DETAILED DESCRIPTION OF THE INVENTION

Herein, convenient screen conditions are provided to increase the positive hits of the antimicrobial screen. Using these conditions, a library of ultrashort peptides (≤10 amino acids) was screened covering the entire LL-37 sequence. The screen identified an eight-residue fragment active against Escherichia coli. New potent peptides based on the newly identified minimal antimicrobial region of LL-37 are also provided.

Antimicrobial screening in Mueller-Hinton broth is frequently the first step in antimicrobial discovery. Although widely utilized, this medium is not ideal as it can mask activity of candidates such as human cathelicidin LL-37 against methicillin-resistant Staphylococcus aureus (MRSA). Herein, a sensitive medium is provided wherein LL-37 displayed excellent activity against numerous pathogens, including MRSA. The screen of ultrashort overlapping LL-37 peptides in this medium led to the identification of KR-8, a potent peptide four residues shorter than KR-12. Hence, the screening condition allows for increasing positive compound hits during antimicrobial screening.

KR-8 also provided a template by which further potent peptides were generated, including LL-37mini, a peptide which was potent against MRSA, Escherichia coli, and Pseudomonas aeruginosa but not toxic to mammalian cells. The peptides of the instant invention have increased antimicrobial potency, a shorter sequence with standard amino acids (thereby making them cheaper to produce), can be tailored for pathogen selectivity, and/or possess antibiofilm efficacy. LL-37mini also inhibited bacterial attachment and biofilm formation and disrupted preformed biofilms in vitro and killed MRSA in murine wound biofilms in vivo. Consistent with membrane targeting, MRSA failed to develop resistance to LL-37mini in a multiple-passage experiment. Notably, LL-37mini can be made cost effectively due to its length and used as a new antibiofilm and antimicrobial agent. The peptides of the instant invention can also serve as a template for engineering other peptide antibiotics such as, without limitation: Trp-cage peptides, cell-penetrating peptides, sidechain coupled stapled peptides, and head-to-tail macrocyclic peptides.

Human cathelicidin LL-37 was also used as a template to design other highly selective ultrashort peptides with similar length, net charge, and hydrophobic content. For example, LL-10+, RK-9+, KR-8+, and RIK-10+ demonstrate similar activity against methicillin-resistant Staphylococcus aureus (MRSA) in vitro and show comparable antibiofilm efficacy in a murine wound model. However, these peptides exhibited varying activity against Gram-negative pathogens, with RIK-10+ (also referred to herein as LL-37mini2) being the most potent and LL-10+ the least. Importantly, the peptides were not toxic to host cells at concentrations much higher than that required to kill bacteria. Notably, the antimicrobial activity of these peptides was influenced by salt (NaCl), pH, and serum. Without being bound by theory, the peptides may primarily kill bacteria by damaging their membranes. The helical structure of one of the most active peptides, LL-37mini2, was confirmed using two-dimensional NMR spectroscopy. Additionally, charge density plots of these peptides revealed that the N-terminal high cationicity is indicative of higher potency. Peptides having a reversed amino acid sequence were also produced. Sequence reversal weakened the activity of RIK-10+ but increased the activity of LL-10+ against resistant strains of Escherichia coli and Pseudomonas aeruginosa. A database search found numerous natural sequences that arrange basic amino acids primarily at the N-terminus. Thus, not only are novel peptide provided herein, but also useful strategies for designing novel antimicrobials (e.g., to control drug-resistant Gram-negative pathogens) are provided.

In accordance with the instant invention, peptides, particularly antimicrobial peptides, are provided. In certain embodiments, the peptide comprises one of SEQ ID NOs: 1-52. The peptides of the instant invention can be used to inhibit, treat, and/or prevent bacterial infections. The peptides of the instant invention can be used to inhibit, treat, and/or prevent bacterial biofilms and/or biofilm formation.

In certain embodiments, the peptide of the instant invention comprises the amino acid sequence X₁RX₂X₃X₄X₅X₆X₇(SEQ ID NO: 47), wherein X₁ is R or K, X₂ is W or I, X₃ is W or V, X₄ is R or Q, X₅ is W or R, X₆ is I or W, and X₇ is R, K, or L. In certain embodiments, SEQ ID NO:47 does not encompass KRIVQRIK (SEQ ID NO: 6). In certain embodiments, X₂ is W. In certain embodiments, X₃ is W. In certain embodiments, X₆ is W. In certain embodiments, X₇ is R or K. In certain embodiments, the peptide comprises KRIVQRIK (SEQ ID NO: 6), KRIWQRIK (SEQ ID NO: 12), KRIWQRWK (SEQ ID NO: 13), KRWWQRWK (SEQ ID NO: 14), KRWWQWWK (SEQ ID NO: 15), RRWWRWWR (SEQ ID NO: 16), or RRWWRWWL (SEQ ID NO: 17). In certain embodiments, the peptide comprises KRIWQRIK (SEQ ID NO: 12), KRIWQRWK (SEQ ID NO: 13), KRWWQRWK (SEQ ID NO: 14), KRWWQWWK (SEQ ID NO: 15), RRWWRWWR (SEQ ID NO: 16), or RRWWRWWL (SEQ ID NO: 17). In certain embodiments, the peptide comprises RRWWRWWR (SEQ ID NO: 16).

In certain embodiments, the peptide has 12 or fewer amino acids, 11 or fewer amino acids, 10 or fewer amino acids, 9 or fewer amino acids, or 8 or fewer amino acids. In certain embodiments, the peptide has 8 or more amino acids, 9 or more amino acids, or 10 or more amino acids. In certain embodiments, the peptide is about 8 to about 12 amino acids in length, about 8 to about 11 amino acids in length, about 8 to about 10 amino acids in length, about 8 or 9 amino acids in length, or 8 amino acids in length.

The amino acid sequence of the peptide of the instant invention may have at least 75%, at least 80%, at least 85%, at least 90%, at least 95%, at least 97%, at least 99%, or 100% homology or identity with any peptide described herein (e.g., SEQ ID NOs: 6, 12, 13, 14, 15, 16, 17, or 47) or any one of the above sequences, particularly at least 90% homology or identity (e.g., the sequence may contain additions, deletions, and/or substitutions). In a particular embodiment, the peptide of the instant invention may extend beyond any peptide described herein (e.g., SEQ ID NOs: 6, 12, 13, 14, 15, 16, 17, or 47) at the amino and/or carboxyl terminus by 1, 2, 3, 4, 5, 6, 7, 8, 9, or 10 amino acids, particularly by 1, 2, 3, 4, or 5 amino acids, by 1, 2, or 3 amino acids, by 1 or 2 amino acids, or by 1 amino acid (e.g., the extension sequence corresponds to the sequence of LL-37 (SEQ ID NO: 1)). In yet another embodiment, the peptides of the instant invention (e.g., SEQ ID NOs: 6, 12, 13, 14, 15, 16, 17, or 47) may also be in reverse orientation (i.e., the sequence from amino terminus to carboxyl terminus is reversed).

In certain embodiments, the peptide comprises the amino acid sequence X₁RWWX₄X₅WX₇ (SEQ ID NO: 48), wherein X₁ is R or K, X₄ is R or Q, X₅ is W or R, and X₇ is R or K. In certain embodiments, X₅ is W. In certain embodiments, X₄ is R. In certain embodiments, X₇ is R. In certain embodiments, X₅ is W and X₄ is R. In certain embodiments, the peptide comprises KRWWQRWK (SEQ ID NO: 14), KRWWQWWK (SEQ ID NO: 15), or RRWWRWWR (SEQ ID NO: 16). In certain embodiments, the peptide comprises RRWWRWWR (SEQ ID NO: 16).

In certain embodiments, the peptide has 12 or fewer amino acids, 11 or fewer amino acids, 10 or fewer amino acids, 9 or fewer amino acids, or 8 or fewer amino acids. In certain embodiments, the peptide has 8 or more amino acids, 9 or more amino acids, or 10 or more amino acids. In certain embodiments, the peptide is about 8 to about 12 amino acids in length, about 8 to about 11 amino acids in length, about 8 to about 10 amino acids in length, about 8 or 9 amino acids in length, or 8 amino acids in length.

The amino acid sequence of the peptide of the instant invention may have at least 75%, at least 80%, at least 85%, at least 90%, at least 95%, at least 97%, at least 99%, or 100% homology or identity with any peptide described herein (e.g., SEQ ID NOs: 14, 15, 16, or 48) or any one of the above sequences, particularly at least 90% homology or identity (e.g., the sequence may contain additions, deletions, and/or substitutions). In a particular embodiment, the peptide of the instant invention may extend beyond any peptide described herein (e.g., SEQ ID NOs: 14, 15, 16, or 48) at the amino and/or carboxyl terminus by 1, 2, 3, 4, 5, 6, 7, 8, 9, or 10 amino acids, particularly by 1, 2, 3, 4, or 5 amino acids, by 1, 2, or 3 amino acids, by 1 or 2 amino acids, or by 1 amino acid (e.g., the extension sequence corresponds to the sequence of LL-37 (SEQ ID NO: 1)). In yet another embodiment, the peptides of the instant invention (e.g., SEQ ID NOs: 14, 15, 16, or 48) may also be in reverse orientation (i.e., the sequence from amino terminus to carboxyl terminus is reversed).

In certain embodiments, the peptide comprises the amino acid sequence LX₁GX₂FFRKX₃K (SEQ ID NO: 49), wherein X₁ is L or W; X₂ is D or R; and X₃ is S or W. In certain embodiments, SEQ ID NO: 49 does not encompass LLGDFFRKSK (SEQ ID NO: 7). In certain embodiments, the peptide comprises LWGRFFRKWK (SEQ ID NO: 30).

In certain embodiments, the peptide has 14 or fewer amino acids, 13 or fewer amino acids, 12 or fewer amino acids, 11 or fewer amino acids, or 10 or fewer amino acids. In certain embodiments, the peptide has 10 or more amino acids, 11 or more amino acids, or 12 or more amino acids. In certain embodiments, the peptide is about to about 14 amino acids in length, about 10 to about 13 amino acids in length, about 10 to about 12 amino acids in length, about 10 or 11 amino acids in length, or amino acids in length.

The amino acid sequence of the peptide of the instant invention may have at least 75%, at least 80%, at least 85%, at least 90%, at least 95%, at least 97%, at least 99%, or 100% homology or identity with any peptide described herein (e.g., SEQ ID NOs: 30 or 49) or any one of the above sequences, particularly at least 90% homology or identity (e.g., the sequence may contain additions, deletions, and/or substitutions). In a particular embodiment, the peptide of the instant invention may extend beyond any peptide described herein (e.g., SEQ ID NOs: 30 or 49) at the amino and/or carboxyl terminus by 1, 2, 3, 4, 5, 6, 7, 8, 9, or 10 amino acids, particularly by 1, 2, 3, 4, or 5 amino acids, by 1, 2, or 3 amino acids, by 1 or 2 amino acids, or by 1 amino acid (e.g., the extension sequence corresponds to the sequence of LL-37 (SEQ ID NO: 1)). In yet another embodiment, the peptides of the instant invention (e.g., SEQ ID NOs: 30 or 49) may also be in reverse orientation (i.e., the sequence from amino terminus to carboxyl terminus is reversed). In certain embodiments, the peptide comprises KWKRFFRGWL (SEQ ID NO: 34), which is the reverse sequence of SEQ ID NO: 30.

In certain embodiments, the peptide comprises the amino acid sequence RX₁X₂KX₃X₄X₅GK (SEQ ID NO: 50), wherein X₁ is K or W; X₂ is S or W; X₃ is E or K; X₄ is K or W; and X₅ is I or W. In certain embodiments, SEQ ID NO: 50 does not encompass RKSKEKIGK (SEQ ID NO: 10). In certain embodiments, the peptide comprises RWWKKWWGK (SEQ ID NO: 31).

In certain embodiments, the peptide has 13 or fewer amino acids, 12 or fewer amino acids, 11 or fewer amino acids, 10 or fewer amino acids, or 9 or fewer amino acids. In certain embodiments, the peptide has 9 or more amino acids, 10 or more amino acids, or 11 or more amino acids. In certain embodiments, the peptide is about 9 to about 13 amino acids in length, about 9 to about 12 amino acids in length, about 9 to about 11 amino acids in length, about 9 or 10 amino acids in length, or 9 amino acids in length.

The amino acid sequence of the peptide of the instant invention may have at least 75%, at least 80%, at least 85%, at least 90%, at least 95%, at least 97%, at least 99%, or 100% homology or identity with any peptide described herein (e.g., SEQ ID NOs: 31 or 50) or any one of the above sequences, particularly at least 90% homology or identity (e.g., the sequence may contain additions, deletions, and/or substitutions). In a particular embodiment, the peptide of the instant invention may extend beyond any peptide described herein (e.g., SEQ ID NOs: 31 or 50) at the amino and/or carboxyl terminus by 1, 2, 3, 4, 5, 6, 7, 8, 9, or 10 amino acids, particularly by 1, 2, 3, 4, or 5 amino acids, by 1, 2, or 3 amino acids, by 1 or 2 amino acids, or by 1 amino acid (e.g., the extension sequence corresponds to the sequence of LL-37 (SEQ ID NO: 1)). In yet another embodiment, the peptides of the instant invention (e.g., SEQ ID NOs: 31 or 50) may also be in reverse orientation (i.e., the sequence from amino terminus to carboxyl terminus is reversed).

In certain embodiments, the peptide comprises the amino acid sequence KRX₁X₂QX₃X₄K (SEQ ID NO: 51), wherein X₁ is I or W; X₂ is V or W; X₃ is R or W; and X₄ is I or W. In certain embodiments, SEQ ID NO: 51 does not encompass KRIVQRIK (SEQ ID NO: 6). In certain embodiments, the peptide comprises KRWWQWWK (SEQ ID NO: 32).

In certain embodiments, the peptide has 12 or fewer amino acids, 11 or fewer amino acids, 10 or fewer amino acids, 9 or fewer amino acids, or 8 or fewer amino acids. In certain embodiments, the peptide has 8 or more amino acids, 9 or more amino acids, or 10 or more amino acids. In certain embodiments, the peptide is about 8 to about 12 amino acids in length, about 8 to about 11 amino acids in length, about 8 to about 10 amino acids in length, about 8 or 9 amino acids in length, or 8 amino acids in length.

The amino acid sequence of the peptide of the instant invention may have at least 75%, at least 80%, at least 85%, at least 90%, at least 95%, at least 97%, at least 99%, or 100% homology or identity with any peptide described herein (e.g., SEQ ID NOs: 32 or 51) or any one of the above sequences, particularly at least 90% homology or identity (e.g., the sequence may contain additions, deletions, and/or substitutions). In a particular embodiment, the peptide of the instant invention may extend beyond any peptide described herein (e.g., SEQ ID NOs: 32 or 51) at the amino and/or carboxyl terminus by 1, 2, 3, 4, 5, 6, 7, 8, 9, or 10 amino acids, particularly by 1, 2, 3, 4, or 5 amino acids, by 1, 2, or 3 amino acids, by 1 or 2 amino acids, or by 1 amino acid (e.g., the extension sequence corresponds to the sequence of LL-37 (SEQ ID NO: 1)). In yet another embodiment, the peptides of the instant invention (e.g., SEQ ID NOs: 32 or 51) may also be in reverse orientation (i.e., the sequence from amino terminus to carboxyl terminus is reversed).

In certain embodiments, the peptide comprises the amino acid sequence RX₁KX₂FLRNX₃V (SEQ ID NO: 52), wherein X₁ is I or W; X₂ is D or R; and X₃ is L or W. In certain embodiments, SEQ ID NO: 52 does not encompass RIKDFLRNLV (SEQ ID NO: 11). In certain embodiments, the peptide comprises RWKRFLRNWV (SEQ ID NO: 33).

In certain embodiments, the peptide has 14 or fewer amino acids, 13 or fewer amino acids, 12 or fewer amino acids, 11 or fewer amino acids, or 10 or fewer amino acids. In certain embodiments, the peptide has 10 or more amino acids, 11 or more amino acids, or 12 or more amino acids. In certain embodiments, the peptide is about 10 to about 14 amino acids in length, about 10 to about 13 amino acids in length, about 10 to about 12 amino acids in length, about 10 or 11 amino acids in length, or 10 amino acids in length.

The amino acid sequence of the peptide of the instant invention may have at least 75%, at least 80%, at least 85%, at least 90%, at least 95%, at least 97%, at least 99%, or 100% homology or identity with any peptide described herein (e.g., SEQ ID NOs: 33 or 52) or any one of the above sequences, particularly at least 90% homology or identity (e.g., the sequence may contain additions, deletions, and/or substitutions). In a particular embodiment, the peptide of the instant invention may extend beyond any peptide described herein (e.g., SEQ ID NOs: 33 or 52) at the amino and/or carboxyl terminus by 1, 2, 3, 4, 5, 6, 7, 8, 9, or 10 amino acids, particularly by 1, 2, 3, 4, or 5 amino acids, by 1, 2, or 3 amino acids, by 1 or 2 amino acids, or by 1 amino acid (e.g., the extension sequence corresponds to the sequence of LL-37 (SEQ ID NO: 1)). In yet another embodiment, the peptides of the instant invention (e.g., SEQ ID NOs: 33 or 52) may also be in reverse orientation (i.e., the sequence from amino terminus to carboxyl terminus is reversed). In certain embodiments, the peptide comprises VWNRLFRKWR (SEQ ID NO: 35), which is the reverse sequence of SEQ ID NO: 32.

The peptides of the instant invention (e.g., SEQ ID NOs: 1-52) may have one or more of the following modifications.

In certain embodiments, the peptide of the instant invention comprises any peptide described herein (e.g., SEQ ID NOs: 1-52) and a terminal cysteine. In a particular embodiment, the cysteine is at the amino terminus. In a particular embodiment, the cysteine is at the carboxyl terminus. The presence of the cysteine provides functionality to allow the covalent linkage of the peptide to, for example, a solid surface (e.g., a medical implant).

As stated hereinabove, the peptide of the instant invention may contain substitutions for the amino acids of the provided sequence. These substitutions may be similar to the amino acid (i.e., a conservative change) present in the provided sequence (e.g., an acidic amino acid in place of another acidic amino acid, a basic amino acid in place of a basic amino acid, a hydrophobic amino acid in place of a hydrophobic amino acid, a polar amino acid for a polar amino acid, etc.). The substitutions may also comprise amino acid analogs and mimetics (e.g., biphenylalanine (e.g., as a substitution for a hydrophobic amino acid)). In certain embodiments, the substitutions are predicted to promote and/or retain helicity or helix formation.

The peptide of the instant invention may have capping, protecting and/or stabilizing moieties at the C-terminus and/or N-terminus. Such moieties are well known in the art and include, without limitation, amidation and acetylation. In a particular embodiment, the peptides of the instant invention are amidated (e.g., at the C-terminus). The peptide template may also be glycosylated at any amino acid (i.e., a glycopeptide). In particular, these peptides may be PEGylated to improve druggability. The number of the PEG units (NH₂(CH₂CH₂O)CH₂CH₂CO) may vary, for example, from 1 to about 50.

The peptide of the instant invention may also comprise at least one D-amino acid instead of the native L-amino acid. In certain embodiments, at least half of the amino acids of the peptide are D-amino acids. The peptide may comprise only D-amino acids. In certain embodiments, the peptides comprise D-amino acids which are spaced apart by about 1, 2, 3, and/or 4 (e.g., 3) consecutive L-amino acids. The inclusion of D-amino acids increases the peptides resistance to proteases.

The peptides of the instant invention may contain at least one derivative of standard amino acids, such as, without limitation, fluorinated residues or nonstandard amino acids (e.g., beta-amino acids).

The peptides of the instant invention may form different structures or forms. Examples of such structures are provided in Lakshmaiah Narayana, et al. (Antibiotics (2024) 13:816; incorporated herein by reference, particularly for peptide structures provided therein). In certain embodiments, the peptide is linear. In certain embodiments, the peptide is circulated head to tail. In certain embodiments, the peptide comprises a loop involving a few residues. In certain embodiments, the peptide is a macrocyclic peptide. In certain embodiments, the peptide is a stapled peptide (e.g., to stabilize helical structure, such as by conjugating or joining i and i+4 or i and i+7).

In certain embodiments, the peptides of the instant invention are lipidated. In certain embodiments, the peptides are conjugated to a lipid (e.g., fatty acids, triglyceride, diglyceride, monoglyceride, phospholipid, and the like), either directly or via a linker. In certain embodiments, the peptides are conjugated to a fatty acid, either directly or via a linker. In certain embodiments, the lipid (e.g., fatty acid) is conjugated to the N-terminus and/or C-terminus of the peptide. In certain embodiments, the lipid (e.g., fatty acid) is conjugated to the N-terminus of the peptide. In certain embodiments, the lipid (e.g., fatty acid) is conjugated to the peptide via an amide bond (e.g., via the reaction of the amine group at the N-terminus of the peptide and the carboxylic acid of the fatty acid). In certain embodiments, the lipid is conjugated via an amine group of the side chain of a lysine (e.g., via an amide bond). The fatty acid can be saturated or unsaturated. In certain embodiments, the fatty acid is saturated. In certain embodiments, the fatty acid is a saturated or unsaturated C4-C20 fatty acid, a C4-C18 fatty acid, a C4-C16 fatty acid, a C6-C14 fatty acid, a C7-C14 fatty acid, a C7-C13 fatty acid, a C8-C12 fatty acid, a C7-C12 fatty acid, a C7-C11 fatty acid, a C8-C11 fatty acid, a C8-C10 fatty acid, a C8 fatty acid, a C9 fatty acid, or a C10 fatty acid. In certain embodiments, the fatty acid is at least 4, 5, 6, 7, 8, 9, 10, 11, 12, 13, or 14 carbons in length. In certain embodiments, the fatty acid is fewer than 14, 13, 12, 11, 10, 9, 8, 7, 6, 5, or 4 carbons in length. In certain embodiments, the fatty acid is a C8 or C10 fatty acid.

The present invention also encompasses compositions comprising at least one peptide of the instant invention and at least one carrier (e.g., a pharmaceutically acceptable carrier). In a particular embodiment, the composition comprises at least two peptides of the instant invention. The present invention also encompasses methods for preventing, inhibiting, and/or treating microbial infections, including microbial/bacterial biofilms. The compositions of the instant invention can be administered to an animal, in particular a mammal (e.g., a human) in order to treat, inhibit, and/or prevent a microbial (e.g., bacterial such as by E. coli, S. aureus, any of the ESKAPE pathogen (including Enterococcus faecium, Staphylococcus aureus, Klebsiella pneumoniae, Acinetobacter baumannii, Pseudomonas aeruginosa, and Enterobacter species), MRSA, etc.) infection (e.g., the composition may be administered before, during, and/or after a microbial infection). The compositions of the instant invention may also comprise at least one other antimicrobial agent (e.g., an antibiotic). The additional antimicrobial agent may also be administered in a separate composition from the peptides of the instant invention. The compositions may be administered at the same time and/or at different times (e.g., sequentially). The composition(s) comprising at least one peptide of the instant invention and the composition(s) comprising at least one additional antimicrobial agent (e.g., an antibiotic) may be contained within a kit.

The peptides of the present invention may be prepared in a variety of ways, according to known methods. In a particular embodiment, the peptides of the instant invention are chemically synthesized. For example, the peptides may be synthesized using a liquid-phase method or solid-phase method. The chemically synthesized peptides may then be purified (e.g., by HPLC).

The peptides may also be purified from appropriate sources (e.g., bacterial, fungal, plant, or animal cultured cells or tissues, optionally transformed) by immunoaffinity purification. The availability of nucleic acid molecules encoding the peptides enables production of the protein using in vitro expression methods and cell-free expression systems known in the art. Peptides may be produced by expression in a suitable prokaryotic or eukaryotic system. For example, a DNA molecule encoding for a peptide may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences. Peptides produced by gene expression in a recombinant prokaryotic or eukaryotic system may be purified according to methods known in the art. A commercially available expression/secretion system can be used, whereby the recombinant protein is expressed and thereafter secreted from the host cell, and readily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemaglutinin epitope. Such methods are commonly used by skilled practitioners.

Peptides of the invention, such as those prepared by the aforementioned methods, may be analyzed and verified according to standard procedures. For example, such peptides may be subjected to amino acid sequence analysis, according to known methods.

Surface coating with antimicrobial peptides is the best option to prevent biofilm formation. Antimicrobial peptides have the advantage of being potent against drug-resistant superbugs and will not leak into the surrounding tissues due to covalent immobilization. The peptides of the instant invention can be covalently immobilized to different surfaces ranging from plastics (e.g., polyethylene terephthalate) to metals (e.g., titanium).

Medical devices or implants comprising at least one peptide of the instant invention are provided, along with methods of making the same. As used herein, the term “medical device” or “medical implant” includes devices, implants, and materials that are permanently implanted and those that are temporarily or transiently present in the patient. In a particular embodiment, at least part of the exposed surface of the medical device or implant is coated with at least one peptide of the instant invention. In a particular embodiment, the medical device or implant comprises a plastic (e.g., polyethylene terephthalate) or a metal (e.g., titanium). In a particular embodiment, the peptide is covalently attached to the surface of the medical implant or device. The peptide may be linked directly (e.g., via a bond) to the surface of the medical device or implant or covalently attached via a linker (e.g., a crosslinker). In certain embodiments, the medical device or implant is a nanofiber or comprises nanofibers to which the peptide is attached.

The instant invention also encompasses methods of synthesizing the coated medical device or medical implant of the instant invention. In a particular embodiment, the method comprises linking a peptide of the instant invention comprising a terminal cysteine to the medical device or medical implant with a sulfhydryl reactive crosslinker (e.g., a maleimide crosslinker). The crosslinker may be reacted with the peptide first, with the medical device or medical implant first, or with both simultaneously. In a particular embodiment, the peptide is linked to a biocompatible polymer (e.g., chitosan) with a sulfhydryl reactive crosslinker (e.g., a maleimide crosslinker), wherein the biocompatible polymer is attached to the medical device or medical implant.

The term “crosslinker” refers to a molecule capable of forming a covalent linkage between compounds. In a particular embodiment, the crosslinker forms a covalent linkage (e.g., a sulfide bond) via the sulfhydryl group of the terminal cysteine of the peptide. Crosslinkers are well known in the art. The cross-linker may be a bifunctional, trifunctional, or multifunctional crosslinking reagent. In a particular embodiment, the crosslinker is non-biodegradable or uncleavable under physiological conditions. In a particular embodiment, the crosslinker is a maleimide crosslinker.

The instant invention also encompasses nucleic acid molecules encoding the peptides of the instant invention. Nucleic acid molecules encoding the peptides of the invention may be prepared by any method known in the art such as, without limitation: synthesis from appropriate nucleotide triphosphates or isolation and/or amplification from biological sources. The availability of nucleotide sequence information enables preparation of an isolated nucleic acid molecule of the invention by oligonucleotide synthesis. Indeed, knowledge of the amino sequence is sufficient to determine an encoding nucleic acid molecule. Synthetic oligonucleotides may be prepared by the phosphoramidite method employed in the Applied Biosystems 38A DNA Synthesizer or similar devices. The resultant construct may be purified according to methods known in the art, such as gel electrophoresis or high-performance liquid chromatography (HPLC).

Nucleic acids of the present invention may be maintained in any convenient vector, particularly an expression vector. Different promoters may be utilized to drive expression of the nucleic acid sequences based on the cell in which it is to be expressed. Antibiotic resistance markers are also included in these vectors to enable selection of transformed cells. Antimicrobial peptide encoding nucleic acid molecules of the invention include cDNA, genomic DNA, RNA, and fragments thereof which may be single- or double-stranded. Thus, this invention provides oligonucleotides having sequences capable of hybridizing with at least one sequence of a nucleic acid molecule of the present invention.

The present invention also encompasses compositions comprising at least one nucleic acid encoding a peptide of the instant invention and at least one carrier (e.g., pharmaceutically acceptable carrier).

As stated hereinabove, the present invention also encompasses compositions comprising at least one peptide of the instant invention and at least one carrier (e.g., pharmaceutically acceptable carrier). The compositions comprising the peptides of the instant invention may be conveniently formulated for administration with any pharmaceutically acceptable carrier(s). For example, the peptides may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents, or suitable mixtures thereof, particularly an aqueous solution. The concentration of the peptide in the chosen medium may be varied and the medium may be chosen based on the desired route of administration of the pharmaceutical composition. Except insofar as any conventional media or agent is incompatible with the peptide to be administered, its use in the pharmaceutical composition is contemplated.

The present invention also encompasses methods for preventing, inhibiting, and/or treating microbial infections (e.g., viral or bacterial). Methods for preventing, inhibiting, and/or treating a biofilm are also encompassed by the instant invention. The method comprises administering at least one peptide of the instant invention (optionally within a composition with a carrier) to the subject and/or biofilm. The method may further comprise administering at least one additional antimicrobial (e.g., antibiotic). In certain embodiments, the microbe is a Gram-negative pathogen. In a particular embodiment, the microbe is an antibiotic-resistant bacteria or an ESKAPE pathogen. In a particular embodiment, the microbe is in a biofilm. In a particular embodiment, the microbe is planktonic. In a particular embodiment, the microbe is selected from the group consisting of Enterococcus faecium, Staphylococcus aureus, Klebsiella pneumoniae, Acinetobacter baumannii, Pseudomonas aeruginosa, and Enterobacter species. In certain embodiments, the microbial infection is a S. aureus infection (e.g., MRSA). The compositions of the instant invention can be administered to an animal, in particular a mammal, more particularly a human, in order to treat, inhibit, and/or prevent a microbial infection (e.g., the composition may be administered before, during, and/or after a microbial infection). The pharmaceutical compositions of the instant invention may also comprise at least one other antimicrobial agent, particularly at least one other antibiotic. The additional antimicrobial agent may also be administered in a separate composition from the antimicrobial peptides of the instant invention. The compositions may be administered at the same time and/or at different times (e.g., sequentially).

In a particular embodiment, the peptides of the instant invention are administered to the subject as a coating on a medical device or implant.

Bacterial infections that may be treated using the present methods include Gram-positive bacterial infections and Gram-negative bacterial infections. In a particular embodiment, the bacteria is a Gram-positive bacteria. In a particular embodiment, the bacteria is a Gram-negative bacteria. In a particular embodiment, the bacteria is a staphylococcal strain. In yet another embodiment, the bacteria is Staphylococcus aureus. More particularly, the bacteria is MRSA. In a particular embodiment, the bacteria is an antibiotic-resistant bacteria or an ESKAPE pathogen. In a particular embodiment, the bacteria is selected from the group consisting of Enterococcus faecium, Staphylococcus aureus, Klebsiella pneumoniae, Acinetobacter baumannii, Pseudomonas aeruginosa, and Enterobacter species.

The peptides described herein will generally be administered to a patient as a pharmaceutical composition. The term “patient” as used herein refers to human or animal subjects. These antimicrobial peptides may be employed therapeutically, under the guidance of a physician.

The dose and dosage regimen of antimicrobial peptides according to the invention that are suitable for administration to a particular patient may be determined by a physician considering the patient's age, sex, weight, general medical condition, and the specific condition for which the antimicrobial peptides are being administered and the severity thereof. The physician may also take into account the route of administration, the pharmaceutical carrier, and the antimicrobial peptide's biological activity.

Selection of a suitable pharmaceutical composition will also depend upon the mode of administration chosen. For example, the antimicrobial peptides of the invention may be administered by direct injection or intravenously. In this instance, a pharmaceutical composition comprises the peptide dispersed in a medium that is compatible with the site of injection.

Peptides of the instant invention may be administered by any method. For example, the peptides of the instant invention can be administered, without limitation by injection, parenterally, subcutaneously, orally, nasally, topically, pulmonarily, rectally, vaginally, intravenously, intraperitoneally, intrathecally, intracerbrally, epidurally, intramuscularly, intradermally, intracarotidly, or other modes of administration such as controlled release devices. In a particular embodiment, the peptides (or compositions) are administered intramuscularly, subcutaneously, or to the bloodstream (e.g., intravenously). In a particular embodiment, the peptides (or compositions) are administered by injection. In a particular embodiment, the peptides (or compositions) are administered topically. The peptides (or compositions) may be directly administered (e.g., by injection) to the site of microbial infection. In general, pharmaceutical compositions and carriers of the present invention comprise, among other things, pharmaceutically acceptable diluents, preservatives, stabilizing agents, solubilizers, emulsifiers, adjuvants and/or carriers. Such compositions can include diluents of various buffer content (e.g., saline, Tris HCl, acetate, phosphate), pH and ionic strength; and additives such as detergents and solubilizing agents (e.g., Tween™ 80, polysorbate 80), anti-oxidants (e.g., ascorbic acid, sodium metabisulfite), preservatives (e.g., Thimersol, benzyl alcohol) and bulking substances (e.g., lactose, mannitol). The compositions can be incorporated into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. Exemplary pharmaceutical compositions and carriers are provided, e.g., in “Remington's Pharmaceutical Sciences” by E. W. Martin (Mack Pub. Co., Easton, Pa.) and “Remington: The Science and Practice Of Pharmacy” by Alfonso R. Gennaro (Lippincott Williams & Wilkins) which are herein incorporated by reference. The compositions of the present invention can be prepared, for example, in liquid form, or can be in pill or dried powder form (e.g., lyophilized).

Pharmaceutical compositions for injection are known in the art. If injection is selected as a method for administering the peptide, steps must be taken to ensure that sufficient amounts of the molecules or cells reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing a peptide of the present invention as the active ingredient in intimate admixture with a pharmaceutically acceptable carrier can be prepared according to conventional pharmaceutical compounding techniques. The carrier may take a wide variety of forms depending on the form of pharmaceutical composition desired for administration, e.g., intravenous, topical, oral, direct injection, intracranial, and intravitreal.

In yet another embodiment, the compositions of the present invention can be delivered in a controlled release system, such as using an intravenous infusion, an implantable osmotic pump, a transdermal patch, liposomes, or other modes of administration. In a particular embodiment, a pump may be used.

A pharmaceutical composition of the invention may be formulated in dosage unit form for ease of administration and uniformity of dosage. Dosage unit form, as used herein, refers to a physically discrete unit of the pharmaceutical composition appropriate for the patient undergoing treatment. Each dosage should contain a quantity of active ingredient calculated to produce the desired effect (e.g., curing, relieving, treating, and/or preventing the microbial infection, the symptoms of it, or the predisposition towards it) in association with the selected pharmaceutical carrier. The dosage should not be so large as to cause adverse side effects, such as unwanted cross-reactions, anaphylactic reactions, and the like. Procedures for determining the appropriate dosage unit are well known to those skilled in the art. Generally, the dosage will vary with the age, weight, condition, sex and extent of the disease in the patient and can be determined by one of skill in the art. Appropriate concentrations for alleviation of a particular pathological condition may be determined by dosage concentration curve calculations, as known in the art. The dosage can be adjusted by the individual physician in the event of any counter indications.

The appropriate dosage unit for the administration of peptides may be determined by evaluating the toxicity of the molecules or cells in animal models. Various concentrations of peptides in pharmaceutical composition may be administered to mice, and the minimal and maximal dosages may be determined based on the beneficial results and side effects observed as a result of the treatment. Appropriate dosage unit may also be determined by assessing the efficacy of the antimicrobial peptide treatment in combination with other standard drugs. The dosage units of antimicrobial peptide may be determined individually or in combination with each treatment according to the effect detected.

The pharmaceutical composition comprising the peptides may be administered at appropriate intervals until the pathological symptoms are reduced or alleviated, after which the dosage may be reduced to a maintenance level. The appropriate interval in a particular case would normally depend on the condition of the patient. In a particular embodiment, the method comprises at least two administrations of the peptides or compositions.

The instant also encompasses delivering the peptides of the instant invention to a cell in vitro (e.g., in culture). The peptide may be delivered to the cell in at least one carrier.

In accordance with another aspect of the instant invention, methods of screening a compound for antibacterial activity are provided. The compound can be any compound such as a nucleic acid, protein or peptide, or small molecule. In certain embodiments, the compound is a peptide. The methods comprise contacting the bacteria with the compound in a diluted media (e.g., diluted culture media) and measuring the cytotoxicity of the compound (e.g., by measuring bacterial growth (e.g., lack thereof) and/or bacterial killing (e.g., compared to a control without the compound)). Prior to the assay with the test compound, the bacteria may be cultured in rich or undiluted media. The media can be any media or tissue culture media that allows for growth of the bacteria. In certain embodiments, the media is selected from the group consisting of Mueller-Hinton Broth (MHB), tryptic soy broth (TSB), and Luria-Bertani (LB; lysogeny broth). In certain embodiments, the media is Mueller-Hinton Broth (MHB). In certain embodiments, the diluted media is 50% or less, 40% or less, 30% or less, 25% or less, 20% or less, 15% or less, 12.5% or less, 10% or less, 7.5% or less, or 5% or less of the undiluted media. In certain embodiments, the diluted media is 10% to 15% of the undiluted media. In certain embodiments, the media is diluted with water.

In accordance with another aspect of the instant invention, methods of designing and/or generating a peptide with antimicrobial activity against Gram-negative bacteria. The method comprises designing a peptide with basic amino acids (R or K) at the N-terminus and a hydrophobicity of at least 40%. In certain embodiments, the peptide has 50 or fewer amino acids, 40 or fewer amino acids, 30 or fewer amino acids, 25 or fewer amino acids, 20 or fewer amino acids, 15 or fewer amino acids, or 10 or fewer amino acids. In certain embodiments, the peptide has 6 or more amino acids, 8 or more amino acids, 10 or more amino acids, or 12 or more amino acids. In certain embodiments, the peptide is about 8 to about 50 amino acids in length, about 8 to about 25 amino acids in length, about 8 to about 20 amino acids in length, about 8 to about 15 amino acids in length, about 8 to about 12 amino acids in length, or about 8 to about 10 amino acids in length. In certain embodiments, at least 50% of the N-terminal four amino acids are charged basic amino acids (R or K, particularly R), particularly at least 75% of the N-terminal four amino acids are charged basic amino acids (R or K, particularly R). In certain embodiments, the hydrophobicity of the peptide (e.g., the percentage of amino acids that are hydrophobic amino acids (A, V, I, L, M, F, W and C)) is at least 50%, at least 60%, or at least 70%. In certain embodiments, the peptide is helical in shape. In certain embodiments, the peptide has a net charge of at least +3 or at least +4.

In certain embodiments, the peptide is designed de novo. In certain embodiments, the peptide is designed from a known antimicrobial peptide (e.g., SEQ ID NOs: 36-46). In certain embodiments, the method comprises inserting and/or substituting charged basic amino acids (R or K, particularly R) into the peptide to satisfy the N-terminal requirement and the overall net charge and inserting and/or substituting hydrophobic amino acids (e.g., W) to satisfy the hydrophobic requirement (e.g., by replacing amino acids (e.g., charged or aliphatic amino acids) with hydrophobic amino acids.

The methods may further comprise synthesizing the peptides. The methods may further comprise testing the synthesized peptide for antimicrobial activity against the Gram-negative bacteria.

### Definitions

The following definitions are provided to facilitate an understanding of the present invention.

The singular forms “a,” “an,” and “the” include plural referents unless the context clearly dictates otherwise.

The term “isolated” is not meant to exclude artificial or synthetic mixtures with other compounds or materials, or the presence of impurities that do not interfere with the fundamental activity, and that may be present, for example, due to incomplete purification, or the addition of stabilizers.

The term “substantially pure” refers to a preparation comprising at least 50-60% by weight of a given material (e.g., nucleic acid, oligonucleotide, protein, etc.). Particularly, the preparation comprises at least 75% by weight, at least 80% by weight, at least 90% by weight, or at least 95% or more by weight of the given compound. Purity may be measured by methods appropriate for the given compound (e.g. chromatographic methods, agarose or polyacrylamide gel electrophoresis, HPLC analysis, and the like).

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween™ 80, polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., microbial (e.g., bacterial) infection) resulting in a decrease in the probability that the subject will develop the condition.

A “therapeutically effective amount” of a compound or a pharmaceutical composition refers to an amount effective to prevent, inhibit, treat, and/or lessen the symptoms of a particular disorder or disease. The treatment of a microbial infection (e.g., a bacterial infection such as a S. aureus infection) herein may refer to an amount sufficient to inhibit microbial growth or kill the microbe and/or curing, relieving, and/or preventing the microbial infection, the symptom of it, or the predisposition towards it.

As used herein, the term “antibiotic” refers to antimicrobial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, the term “subject” refers to an animal, particularly a mammal, particularly a human.

The term “promoter” as used herein refers to a DNA sequence which directs transcription of a polynucleotide sequence operatively linked thereto (e.g., in a cell). The promoter may also comprise enhancer elements which stimulate transcription from the linked promoter. The term “enhancer” refers to a DNA sequence which binds to the transcription initiation complex and facilitates the initiation of transcription at the associated promoter.

A “vector” is a nucleic acid molecule such as a plasmid, cosmid, bacmid, phage, or virus, to which another genetic sequence or element (either DNA or RNA) may be attached/inserted so as to bring about the replication and/or expression of the sequence or element (e.g., under the control of a promoter contained within the vector).

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. An isolated nucleic acid (either DNA or RNA) may further represent a molecule produced directly by biological or synthetic means and separated from other components present during its production.

“Linker” refers to a chemical moiety comprising a covalent bond or a chain of atoms that covalently attach at least two compounds. The linker can be linked to any synthetically feasible position of the compounds, but preferably in such a manner as to avoid blocking the compounds desired activity. Linkers are generally known in the art. In a particular embodiment, the linker may contain from 0 (i.e., a bond) to about 50 atoms, from 0 to about 10 atoms, or from about 1 to about 5 atoms.

The term “antimicrobials” as used herein indicates a substance that kills or inhibits the growth of microorganisms such as bacteria, fungi, viruses, or protozoans, particularly bacteria.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The following examples describe illustrative methods of practicing the instant invention and are not intended to limit the scope of the invention in any way.

### Example 1

### Materials and Methods

### Chemicals and Peptides

All of the chemicals were purchased from established vendors such as Fisher and Sigma. Peptides were made by Genemed Synthesis, Inc. (San Antonio, TX). All of the peptides were highly purified and reached over 95% purity based on high-performance liquid chromatography (HPLC). The correct mass of each peptide was validated by mass spectrometry. Peptides were quantitated by measuring UV absorbances at 280 nm (with W) or at 215 and 225 nm (without W) based on the Waddell's method (Waddell, W. J. (1956) J. Lab. Clin. Med., 48:311-314).

### Antibacterial Assay

The following resistant bacterial clinical strains were used in this study: S. aureus USA300 LAC, S. epidermidis 1457, E. coli E423-17, P. aeruginosa E411-17, K. pneumoniae E406-17, and A. baumannii B28-16. While the S. aureus USA300 strain is methicillin-resistant, most of the Gram-negative bacteria contain extended-spectrum beta-lactamase and are resistant to beta-lactam antibiotics. Peptide activity against bacteria was tested using an established lab protocol (Mishra, et al. (2017) Acta Biomater., 49:316-328). In brief, a peptide concentration gradient with twofold dilution was made in the 96-well polystyrene microplate at 10 μL per well. Bacteria were grown to the exponential phase (i.e., optical density at 600 nm≈0.5), diluted to ˜10⁵ CFU/mL in different percentages of MHB, TSB, or LB, and partitioned into the 96-well microplate at 90 μL per well. Rich media prepared as suggested by the vendors are defined as 100% in this study. They were diluted with autoclaved distilled water to obtain different percentages. The microplates were incubated at 37° C. overnight and read on a ChroMate™ 4300 microplate reader at 600 nm (GMI, Ramsey, MN). The peptide concentration in the wells without bacterial growth is the MIC. As is the convention, MIC values were represented as ranges.

For pH effects on peptide activity, the pH of the medium was adjusted to a targeted pH for the autoclave and then remeasured at room temperature. For salt and serum effects on peptide activity, stock NaCl solution or human serum was added to the media.

### Peptide Protease Stability

Protease stability of the peptides was tested in the presence and absence of mammalian trypsin, chymotrypsin, and pepsin (Thermo Fisher Scientific, MA) based on modified antimicrobial assays, which give a similar conclusion as HPLC-based stability assays (Decker, (2023) Mol. Pharmaceutics 20:738-749). A solution of peptide/protease (40:1 molar ratio) was made in phosphate-buffered saline (PBS; pH 7.1, Gibco, NY) and incubated at 37° C. for 1 hour. As controls, peptides without proteases were also incubated in the same manner. Then, aliquots of 10 μL were transferred to a 96-well polystyrene microplate and mixed with the exponential-phase MRSA USA300 culture as performed in the antibacterial activity assays above. If the peptide is stable, then no growth is anticipated at and above the MIC.

### Killing Kinetics

MRSA USA300 was grown to the exponential phase (OD₆₀₀˜0.3) in MHB. The culture was diluted to OD₆₀₀ 0.001 (˜10⁵ CFU/mL). One milliliter of bacterial suspension was then mixed with LL-37mini or daptomycin at 2×MIC and incubated at 37° C. After 15, 30, 60, 90, and 120 minutes of incubation, 50 μL was taken and serially diluted with 1×PBS. Then, 50 μL of the diluted suspension was plated on mannitol salt agar plates (NEOGEN, MI). The plates were incubated overnight at 37° C. for bacterial CFU determination the next day.

### Hemolytic Assay

The hemolytic effects of peptides were evaluated using a method with minor modifications (Lakshmaiah Narayana, et al. (2021) ACS Infect. Dis., 7:1795-1808). Human red blood cells (hRBCs, UNMC Blood Bank) were washed (3×) with blood bank saline (isotonic solution 0.90% w/v, Fisher). Cells were diluted to 2% using the saline solution, and aliquots of 90 μL were added to a 96-well polystyrene microplate containing 10 μL of serially diluted peptide solutions. After incubation at 37° C. for 1 hour, plates were centrifuged at 500 g for 5 minutes. Aliquots of the supernatant were transferred to a 96-well microplate. The amount of hemoglobin released was measured at 545 nm by using a ChroMate™ microplate reader. To calculate the percent lysis, it was assumed that 100% of the hemoglobin was released when hRBCs were treated with 1% Triton® X-100, while incubation with water resulted in 0% release. HC₅₀ is defined as the concentration of the peptide that caused 50% lysis.

### Cell Viability Assay

Human keratinocyte HaCaT cells were grown in DMEM/high glucose (Hyclone, UT) containing 10% fetal bovine serum (Mediatech, Corning, Manassas, VA). Cells were grown at 37° C. in 5% CO₂, and the medium was changed every other day. Peptides were diluted in a 96-well polystyrene microplate (10 μL each well) and mixed with 90 μL of cell suspensions (10⁵ cells/mL) in their respective media. The mixture was incubated at 37° C. in 5% CO₂ for 24 hours. Then, 20 μL of MTS reagent (MTS cell proliferation assay kit, Promega, WI) was added to each well and incubated at 37° C. for 2 hours. The absorbance was determined at 492 nm using a microplate reader. The percentage viability was determined using the following formula: percentage viability=(OD value of treated cells/OD value of untreated cells)×100, where OD is the optical density.

### Membrane Permeabilization

The peptide ladder was prepared as described for the antibacterial assay with 10 μL of solution in a black COSTAR® 96-well plate. PI (MP Biomedicals, Solon, OH) was prepared in the dark and dissolved in dimethylsulfoxide (Thermo Fisher Scientific, NY) to 20 mM. This PI stock solution was further diluted to 1 mM with water, and 2 μL of 1 mM PI was added to each well. Exponential-phase MRSA USA300 was diluted to OD₆₀₀ 0.11 with TSB, and 88 μL was added to each well. The plate was incubated at 37° C. with continuous shaking in a FLUOstar® Omega (BMG LABTECH, NC) microplate reader. The sample was read every 5 minutes for 24 cycles with excitation and emission wavelengths of 584 and 620 nm, respectively.

### Membrane Depolarization

An overnight culture of MRSA USA300 was inoculated into TSB and grown to an exponential phase. Bacteria were washed with 1×PBS, resuspended in twice the volume of 1×PBS containing 25 mM glucose, and incubated at 37° C. for 15 minutes. Then, 500 nM (final concentration) of DiBAC4 (3) bis(1, 3-dibutylbarbituric acid) trimethine oxonol (ANASPEC, CA) was added, and the mixture was vortexed gently. Aliquots of 90 μL of the energized bacteria solution were loaded into the 96-well plates (Corning COSTAR, AZ) and placed in a FLUOstar® Omega microplate reader (BMG LABTECH, NC). Fluorescence was read for 20 minutes at excitation and emission wavelengths of 485 and 520 nm, respectively. Then, 10 μL of peptide solutions was added, and fluorescence readings were recorded for 40 minutes. Triton® X-100 (0.1%) was used as the positive control.

### Scanning Electron Microscopy

The exponential-phase S. aureus USA300 LAC was treated with a 2×MIC of LL-37mini for 30 minutes. Samples were washed three times with 1×PBS and fixed with 2% paraformaldehyde and 2% glutaraldehyde in 0.1 M PBS. Next, the samples were washed with Sorensen's buffer and postfixed in 1% osmium tetroxide in water for 30 minutes. All samples were then washed in Sorensen's buffer and dehydrated through a graded ethanol series (50, 70, 90, 95, and 100% with ×3 changes). Subsequently, samples were placed in bis(trimethylsilyl) amide (HIDS) 100% for 10 minutes with three changes and left in HMDS in open dishes in the fume hood overnight to allow the HMDS to evaporate. The following day, the samples were mounted on 25 mm aluminum SEM stubs with carbon adhesive tabs. Silver paste was placed around the edges of the samples. Samples were sputter coated with 50 nm of gold/palladium in a Hummer VI sputter coater (Anatech Ltd., Battle Creek, MI) and examined in an FEI Quanta 200 SEM at the UNMC Electron Microscopy Core Facility.

### Bacterial Resistance Development

The experiment was conducted as described (Lakshmaiah et al. (2020) Proc. Natl. Acad. Sci., 117:19446-19454). Briefly, MRSA USA300 was grown to the exponential phase in MHB and diluted to OD₆₀₀ 0.001 (˜10⁵ CFU/mL). Aliquots of 90 μL were added to a 96-well polystyrene microplate (Costar, Corning, NY) containing 10 μL of serially diluted LL-37mini, nafcillin, or daptomycin. After overnight incubation at 37° C., the plates were read at 600 nm using a ChroMate™ microplate reader (GMI, Ramsey, MN). Untreated bacterial culture and medium were included as untreated control and blank, respectively. The MIC is the lowest peptide concentration that inhibited the bacterial growth. The wells with sub-MIC levels of the peptides that retained growth approximately half the growth of the control wells were reinoculated in fresh MHB with sub-MIC concertation of peptides or antibiotics to attain exponential phase for MIC determination. In total, 17 passages of the bacterial cultures were conducted. A rapid increase in MIC is an indication of antibiotic resistance.

### In Vitro Antibiofilm Activity of LL-37mini

Inhibition of Bacterial Attachment. Bacterial attachment is the initial step for biofilm formation. For this experiment, an overnight culture of MRSA USA300 was grown overnight in TSB media to an optical density (OD₆₀₀) of ˜1.0. Then, 180 μL of this culture was added to each well of the microtiter plates containing 20 μL of various MIC folds (1×, 2×, 4×, and 8×MIC) of LL-37mini or daptomycin solutions. The plates were then incubated at 37° C. for 1 hour. Next, the medium was aspirated and washed with 1×PBS, and 200 μL of TSB containing XTT 10% [2,3-bis(2-methyloxy-4-nitro-5-sulfophenyl)-2H-tertazolium-5-carboxanilide](ATCC, VA) solution was added. After incubation at 37° C. for 2 hours, absorbance was read at 450 nm using a ChroMate™ microtiter plate reader. TSB containing 10% XTT served as a blank, while bacterial culture treated with water served as a positive control. Percentages of viable cells in biofilms were plotted by assuming 100% growth in water treated control.

Biofilm Inhibition. MRSA USA300 (10⁵ CFU/mL) was prepared from the exponential phase. Aliquots of 180 μL of bacterial suspension in TSB were added to each well of the microtiter plates containing 20 μL of various MIC folds (0.5 to 2×MIC) of LL-37mini or daptomycin solutions and incubated at 37° C. for 24 hours. Media were then pipetted out, and the wells were washed with 1×PBS to remove planktonic cells. Live cells in biofilms were quantitated by XTT as described above.

Effects of Peptides on Established Biofilms in Vitro. Bacteria (S. aureus USA300 LAC and E. coli E423-17) were grown in rich TSB media overnight. A second inoculation was made the next day to reach an exponential phase (OD₆₀₀˜0.4). Microtiter plates (96 wells, Corning Costar Cat no. 3595), after aliquoting with 180 μL of the culture to each well, were incubated at 37° C. for 24 hours to form biofilms in rich TSB. Media were aspirated post incubation, and the attached biofilms were washed with 1×PBS to remove the planktonic bacteria. Each well was aliquoted with 20 μL of 10× peptide solution and 180 μL of 10% fresh MHB, and plates were further incubated at 37° C. for 24 hours. Biofilms treated with water served as the positive control, while media without bacterial inoculation served as the negative control. Live cells in the biofilms were quantitated using XTT as described above. Absorbance was read at 450 nm (only media with XTT containing wells served as the blank) using a ChroMate™ microtiter plate reader. Percentage biofilm growth for the peptide was plotted by assuming 100% biofilm growth in bacterial control alone. The data were represented as mean±SD; plots were generated using GraphPad prism 7, where * indicates p<0.05, **p<0.01, and ***P<0.001, and ****p<0.0001 (one-way analyses of variance).

### In Vivo Antibiofilm Activity of LL-37mini

Preparation of Inoculum. An overnight culture of S. aureus USA300 LAC was inoculated in fresh MHB and incubated at 37° C. for 3 hours. The suspension was centrifuged at 3000 g for 5 minutes, and the supernatant was discarded. The bacteria were resuspended in 1×PBS, and the OD₆₀₀ was adjusted to 0.6 (˜3×10⁷ CFU/mL).

Experimental Animals. An alternative murine wound model has been established which is slightly different from other studies (Su, et al. (2019) Mol. Pharmaceutics 16:2011-2020; Su, et al. (2023) J. Controlled Release 356:131-141). Female BALB/c mice (3-4 weeks, ˜20 g) were fed with standardized food (Teklad Laboratory diet for rodents) and water (Hydropac Alternative Watering System) ad libitum. Mice were kept in ventilated cages (IVCs) at a temperature of 20-24° C., a humidity of 50-60%, 60 air exchanges per hour, and a 12/12 hour light/dark cycle. All materials, including IVCs, lids, feeders, bottles, bedding, and water, were autoclaved before use. All animal manipulations were performed in a class II laminar flow biological safety cabinet. The study was approved by the Institutional Animal Care and Use Committee (IACUC) of UNMC (Protocol no. 22-015-08-FC).

In Vivo Biofilm Assay. Mice were anesthetized by intraperitoneal injection of ketamine-xylazine (100 mg/kg+10 mg/kg). The dorsal back hair was removed with a clipper and depilatory cream, followed by cleaning the shaved area with isopropyl alcohol (70%) and povidone iodine swabs. Two full-thickness skin wounds with a diameter of 6 mm were then created by using a disposable biopsy punch (Integra Miltex, MA). The wounds were immediately inoculated with MRSA USA300 (10 μL of ˜3×10⁷ CFU/mL), and wounds were covered with a transparent film dressing (3 M Tegaderm, Deutschland GmbH, Neuss, Germany). Buprenorphine (0.5 mg/kg, SC) was injected, and mice were kept in individual cages. After 24 hours of inoculation and biofilm formation, treatment groups received 3×10 μL of LL-37mini or daptomycin dissolved in sterile water. Treatment was applied topically on the wound, and wounds were covered with the transparent film dressing. The infected control group received an equal volume of sterile water. After 24 hours from the time of treatment, mice were euthanized under CO₂ and specimens were collected using sterile 8 mm biopsy punch (Integra Miltex, MA) into sterilized tubes containing 2 mL of PBS. Tissues were blended using a homogenizer, diluted further in 1×PBS, and plated on mannitol salt agar plates. Finally, the plates were incubated at 37° C. for 20 hours and the CFUs were counted.

Statistical Analysis. Data were analyzed using GraphPad Prism 7 (GraphPad Software, Inc., San Diego, CA), and values were expressed as the mean±standard deviation. One-way analyses of variance was used to compare the mean values among treatment groups. P<0.05 was considered statistically significant.

### Results

Identification of a Medium Condition to Reveal the Antibacterial Activity of LL-37 Against S. aureus USA300

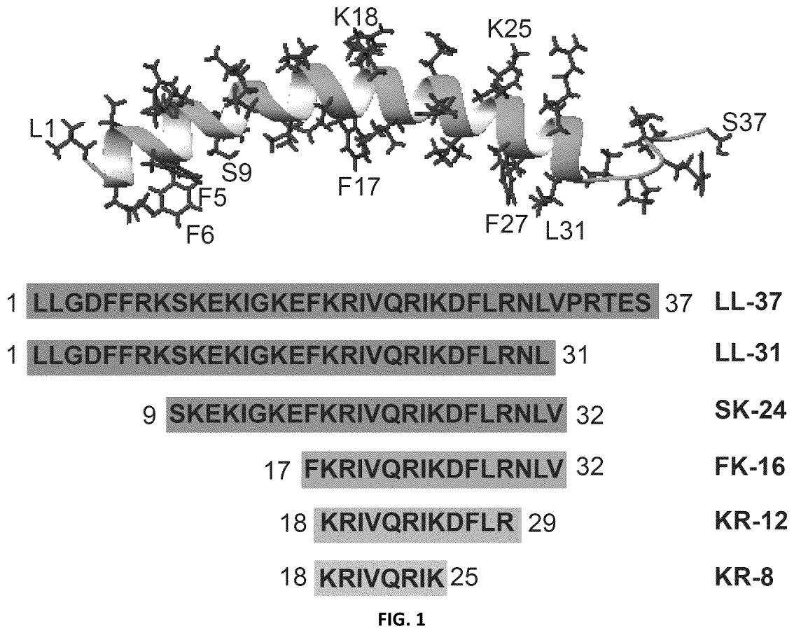

To identify a useful medium condition, the antibacterial activity of LL-37 was compared in several frequently used rich media: NMB, tryptic soy broth (TSB), and Luria-Bertani (LB). These rich media were made as recommended and were defined as 100% in this study. As anticipated, the inhibition of methicillin-resistant S. aureus (MRSA) USA300 LAC by LL-37 was not observed in all these media until 32 μM (Table 1). As a positive control, bacterial killing was observed for 17BIPHE2, a peptide engineered based on the core major AMP FK-16 of human LL-37 (amino acid sequences in FIG. 1) by changing 120, 124, and L28 into D-leucine and F17 and F27 to biphenylalanine (Wang, et al. (2014) ACS Chem. Biol., 9:1997-2002).

TABLE 1
Antimicrobial activity (micromolar) of LL-37
and 17BIPHE2 in different media.

| | | S. aureus USA300 | | E. coli E423-17 | |
|---|---|---|---|---|---|
| | | LL-37 | 17BIPHE2 | LL-37 | 17BIPHE2 |
| MHB | 100% | >32 | 4 | 8 | 2-4 |
| | 50% | 32 | 4 | 4-8 | 2 |
| | 25% | 8 | 4 | 4 | 4 |
| | 12.5% | 4 | ≤2 | 2-4 | 2-4 |
| TSB | 100% | >32 | 2-4 | 8 | 4-8 |
| | 50% | >32 | ≤2 | 4-8 | 4 |
| | 25% | >32 | ≤2 | 4 | 2 |
| | 12.5% | >32 | ≤2 | ≤2 | <2 |
| LB | 100% | >32 | ≤2 | 4 | 2-4 |
| | 50% | >32 | ≤2 | 4-8 | 2-4 |
| | 25% | >32 | 4 | 4 | 2 |
| | 12.5% | >32 | 4 | <2 | ≤2 |

The anti-MRSA activity of LL-37 was then tested in diluted media. Interestingly, LL-37 inhibited the growth of MRSA with the dilution of MHB but not the dilution of TSB nor LB (Table 1). As a positive control, the minimal inhibitory concentration (MIC) of 17BIPHE2 was barely compromised from 100 to 12.5% (2-4 μM). Importantly, the MIC (4 μM) value for LL-37 in 12.5% MHB was comparable to that of 17BIPHE2. Further antimicrobial assays revealed that such an enhanced activity in diluted media was also observed for Staphylococcus epidermidis, Pseudomonas aeruginosa, and Klebsiella pneumoniae (Table 2). Notably, media dilution had only a subtle effect on the antibacterial activity of LL-37 against E. coli (Table 1) and A. baumannii (Table 2). In all of the cases, LL-37 displayed an optimal activity in 12.5% NMB. Remarkably, 17BIPHE2 displayed consistent MIC values under all of the conditions tested (media, dilution, and bacteria), indicative of its antimicrobial robustness (Tables 1 and 2).

TABLE 2
Media dilution effect on antimicrobial activity (micromolar)
of LL-37 and 17BIPHE2 against other bacteria.

| Media | S. epidermidis 1457 | |
|---|---|---|
| MHB | LL-37 | 17BIPHE2 |
| 100% | >32 | 4 |
| 10% | 2-4 | 4 |
| | P. aeruginosa 152 | |
| MHB | LL-37 | 17BIPHE2 |
| 100% | 32 | 4 |
| 10% | 4 | ≤2 |
| | K. pneumonia E406-17 | |
| MHB | LL-37 | 17BIPHE2 |
| 100% | 16-32 | 2-4 |
| 10% | 4 | 4 |
| | A. baumannii B28-16 | |
| MHB | LL-37 | 17BIPHE2 |
| 100% | 8 | ≤2 |
| 10% | 4 | 4 |

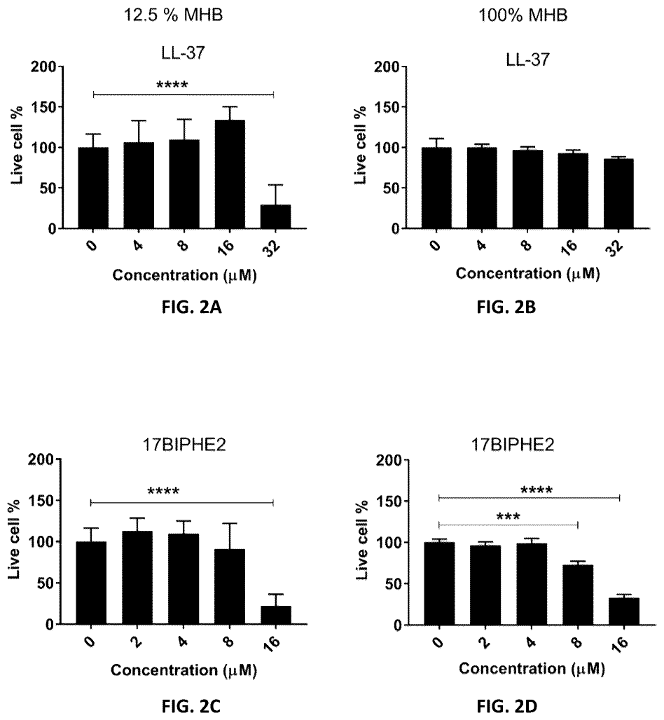

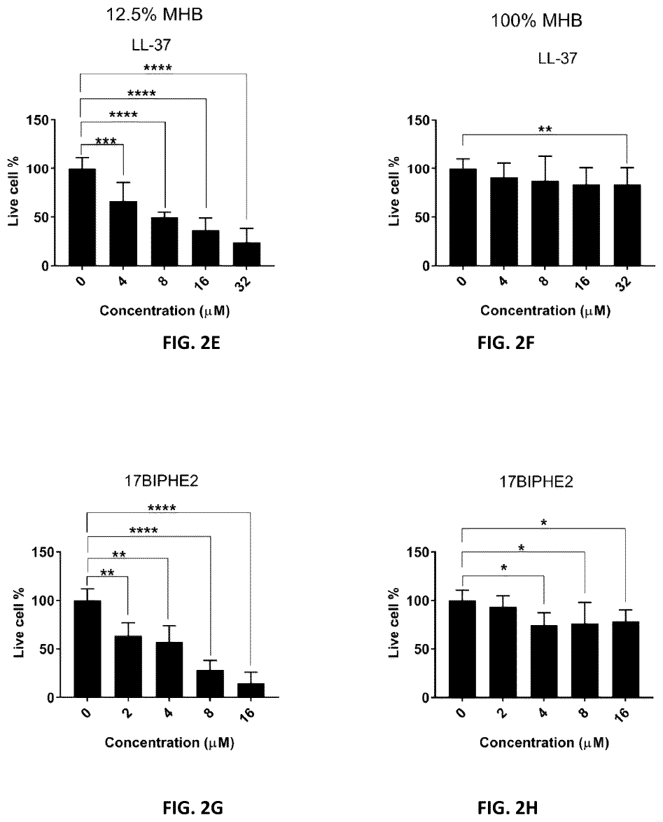

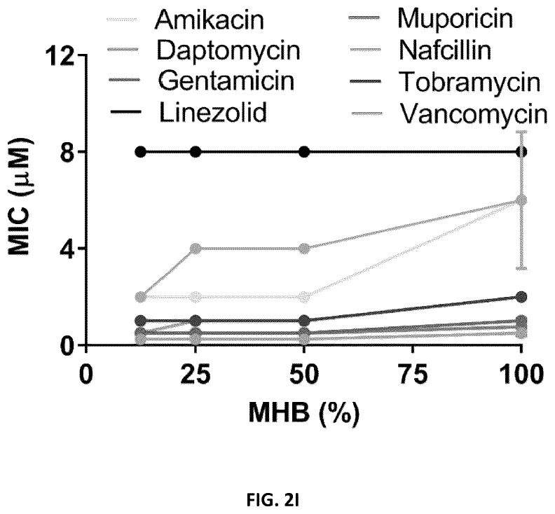

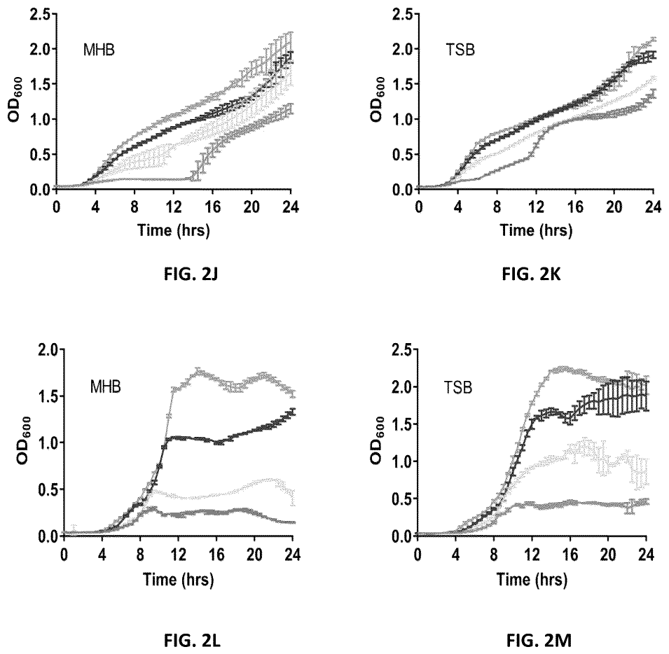

LL-37 is known to inhibit bacterial biofilms (Overhage, et al. (2008) Infect. Immun., 76:4176-4182). However, it was unable to disrupt preformed MRSA biofilms in rich media (Mishra, et al. (2016) ACS Med. Chem. Lett., 7:117-121). Next, it was determined whether the diluted MHB medium could be useful to detect biofilm-disrupting activity for LL-37 against MRSA. An established procedure (Mishra, et al. (2016) ACS Med. Chem. Lett., 7:117-121; Duplantier, et al. (2013) Front. Immunol., 4:143) was used wherein biofilms were formed and washed as usual to remove planktonic cells. The biofilms were then treated with peptides in 12.5% MHB for 24 hours. Finally, live cells were quantified with XTT (ATCC). FIG. 2 compares the levels of live cells of MRSA in the preformed biofilms after 24 hour treatment. In 100% MHB, LL-37 showed essentially no antibiofilm activity, while 17BIPHE2 started to work at 8 μM and only ˜30% live bacteria remained in the biofilms when treated at 16 μM. Of note is a similar pattern for both peptides in 12.5% MHB treated at various concentrations. LL-37 worked at 32 μM, while 17BIPHE2 showed clear biofilm disruption at 16 μM. Hence, similar antibiofilm activity for LL-37 and 17BIPHE2 was observed in this medium as well.

The antibiofilm activity of LL-37 and 17BIPHE2 against clinical E. coli strain E423-17 was compared. In 100% MHB, both LL-37 and 17BIPHE2 showed a subtle effect on biofilms with the increase in peptide concentration. However, a dose-dependent antibiofilm effect is clear in 12.5% MHB (FIGS. 2E-2H). These results indicate that 12.5% MHB (used only during treatment) is also useful for antibiofilm assays to avoid activity masking. This media dilution benefit appears to be media-dependent as a similar trend was not observed for LL-37 when TSB- and LB-rich media was diluted in the same manner (Table 1).

The effects of MHB dilution on the antimicrobial activity of a panel of antibiotics was also compared (FIG. 2I). For nafcillin and amikacin, small changes in MIC values were observed with the dilution of MHB from 100 to 12.5%, implying an effect of media on their activity. However, there were essentially no changes in the antibacterial activity for daptomycin, gentamicin, linezolid, muporicin, tobramycin, and vancomycin with the dilution of MHB in the same manner (FIG. 2I). Since the majority of antibiotics (75%) showed similar MIC values with MHB dilution, these results underscore the usefulness of the diluted MHB for antimicrobial screening. Since bacterial mass will reduce with the dilution of the MHB media (FIGS. 2J-2M), it is important not to dilute the media to the extent where bacteria do not grow or differences in bacterial growth are no longer clear.

### Application of the Diluted MHB Medium for Antimicrobial Peptide Screen

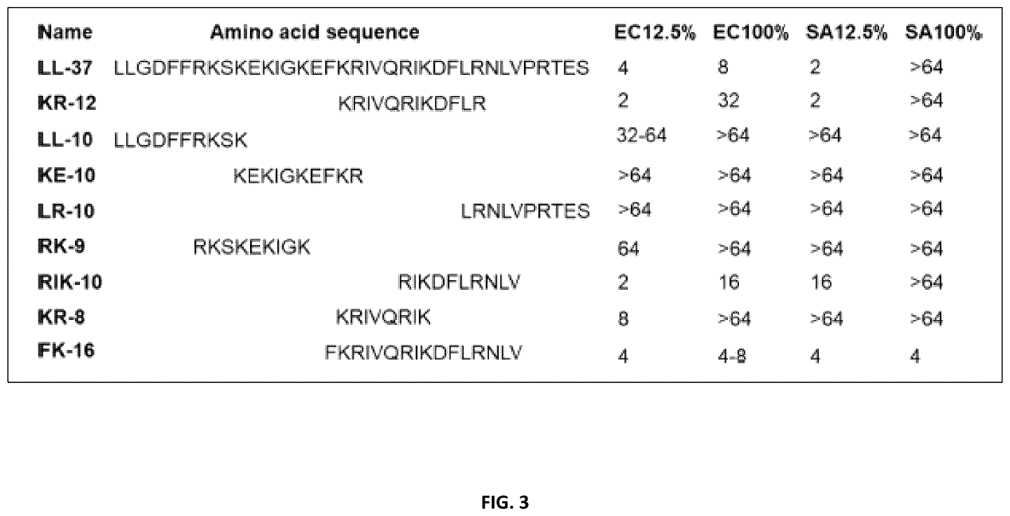

It was hypothesized that antimicrobial agents could better display their activity in the diluted NMB medium, thereby increasing positive hits during the antimicrobial screen. To illustrate this, a small library of overlapping LL-37 peptides with 8-10 amino acids was synthesized, covering the entire length of LL-37 (FIG. 3). These fragments are all shorter than KR-12 (12 residues), the smallest antibacterial peptide against E. coli identified in rich (100%) media (Wang, G. (2008) J. Biol. Chem., 283:32637-32643). In 100% MHB, all of these shorter peptides, including LL-37 and KR-12, did not display any activity against S. aureus USA300 (FIG. 3). In the case of E. coli, LL-37 and KR-12 were active. Most of the ultrashort peptides were inactive in rich media (MIC>64 μM). RIK-10, however, emerged as a new peptide that inhibited the growth of E. coli in rich media (MIC 16 μM). RIK-10 is a 10-residue peptide corresponding to residues 23-32 of LL-37 in FIG. 3. It is named RIK-10 here to distinguish it from the inactive RI-10 peptide (corresponding to residues 19-28 of LL-37). In 12.5% MHB, however, LL-37, KR-12, and RIK-10 all became active against S. aureus USA300. In the same diluted medium, KR-8 (an eight-residue LL-37 peptide starting with KR) became active against E. coli (MIC 8 μM) but not against S. aureus (MIC>64 μM). In addition, very weak activity for LL-10 and RK-9 was observed against E. coli (MIC=32-64 μM) (FIG. 3). To further validate the screening condition, the antimicrobial activity of these LL-37 peptides in RPMI and Dulbecco's modified Eagle medium (DMEM) was compared (Table 5). LL-10, KE-10, LR-10, and RK-9 were not active (MIC>64 μM). In DMEM, RIK-10 was active against E. coli (MIC=8 μM), although it showed poor activity in RPMI or against S. aureus. KR-8 showed only poor activity against E. coli in DMEM. LL-37 and KR-12 remained active in both RPMI and DMEM against S. aureus and E. coli. Collectively, similar trends were obtained for these LL-37 peptides in diluted MHB or cell-culturing media. Such a picture is consistent with the finding that the C-terminus of LL-37 is disordered and not involved in bacterial killing, while the N-terminus of LL-37 is weakly active (Li, et al. (2006) J. Am. Chem. Soc., 128:5776-5785). The major antimicrobial region is located in the central region of LL-37.

Interestingly, KR-8 and RIK-10 are both derived from the major antimicrobial region of human LL-37 identified via NMR studies (FK-16, FIG. 3) (Li, et al. (2006) J. Am. Chem. Soc., 128:5776-5785). An N-terminally glycine-appended FK-16 called GF-17 is active against both E. coli and S. aureus in both rich and diluted NMB media (FIG. 3). The addition of a glycine at the N-terminus has a minimal impact on the peptide since FK-16 and GF-17 have similar antimicrobial potency (Wang, G., et al. Advances in Experimental Medicine and Biology; Springer, 2019; Vol. 1117, pp 215-240). Like the activity spectrum of KR-12 in rich media, KR-8 was also active against E. coli but not against S. aureus in 12.5% MHIB, identifying an even shorter anti-E. coli region within LL-37 (FIG. 3). This KR-8 peptide (residues 18-25 of LL-37) identified in a more sensitive medium is four residues shorter than KR-12 (residues 18-29 of LL-37).

### Ultrashort Peptide KR-8 Provides a Template for Engineering LL-37 Mini

Since KR-8 (i.e., W538 in Table 3) is the shortest active template, peptide activity was sought to be increased. A series of peptides (W538-W544) were designed (Table 3) by replacing aliphatic amino acids with fused aromatic residues. Select peptide parameters such as net charge and hydrophobic percentage are provided in Table 3, and additional parameters are listed in Table 4. Antibacterial activity of these peptides is also listed in Table 3. After the deployment of three tryptophan (W) residues, activity for W541 was first observed against E. coli (MIC 8 μM). When one more W was used to substitute R23 (as named in LL-37, FIG. 1), the resulting peptide W542 gained activity against S. aureus and P. aeruginosa. Due to a preferred association between W and R in Trp-rich peptides (Mishra, et al. (2017) Acta Biomater., 49:316-328), an arginine-rich peptide was generated by changing Q22 to R22 and two K to R. These changes had only a subtle effect on peptide activity. Also, the peptide became active against additional strains K. pneumoniae and A. baumannii when the C-terminal residue was replaced with a leucine residue. Since the net charges of these peptides are relatively constant, it is the increase in hydrophobic amino acids of the KR-8 template to 50% that was critical to enhance peptide antibacterial activity against S. aureus USA300, E. coli 423-17, and P. aeruginosa 423-17 (Table 3). In addition, the increase in peptide hydrophobic ratio from W541 (38%, only E. coli), W543 (50%, three bacteria), to W544 (63%, five bacteria) proportionally increased the activity spectrum of the peptides (Table 3).

TABLE 3
Enhancement of the activity (micromolar) of KR-8 into LL-37mini with
eight amino acids. All the peptides are C-terminally amidated. Q: net charge;
Pho: hydrophobic content; KR-8: W538 in this series; and W543: LL-37mini.

| | amino acid | | | | |
|---|---|---|---|---|---|
| peptide | sequence | SEQ ID NO: | Q | Pho | S. aureus |
| W538 | KRIVQRIK | 6 | +5 | 38% | >32 |
| W539 | KRIWQRIK | 12 | +5 | 38% | >32 |
| W540 | KRIWQRWK | 13 | +5 | 38% | >32 |
| W541 | KRWWQRWK | 14 | +5 | 38% | >32 |
| W542 | KRWWQWWK | 15 | +4 | 50% | 8 |
| W543 | RRWWRWWR | 16 | +5 | 50% | 4-8 |
| W544 | RRWWRWWL | 17 | +4 | 63% | 4 |

| peptide | E.coli | P. aeruginosa | K. pneumoniae | A. baumannii |
|---|---|---|---|---|
| W538 | >32 | >32 | >32 | >32 |
| W539 | >32 | >32 | >32 | >32 |
| W540 | >32 | >32 | >32 | >32 |
| W541 | 8 | >32 | >32 | >32 |
| W542 | 4 | 4-8 | >32 | 32 |
| W543 | 8 | 8 | >32 | >32 |
| W544 | 4 | 4 | 8-16 | 8-16 |

TABLE 4
Physical parameters of KR-8 derived peptides. WWH: Wimley-White
whole-residue hydrophobicity (i.e., the sum of whole-residue free energy of transfer
of the peptide from water to POPC interface); GRAVY: the grand average hydropathy
value of the peptide. Calculations were conducted using the tool in the Antimicrobial
Peptide Database (aps.unmc.edu/prediction).

| | amino acid | | | | Boman index |
|---|---|---|---|---|---|
| peptide | sequence | SEQ ID NO: | WWH | GRAVY | (kcal/mol) |
| W538 | KRIVQRIK | 6 | 3.63 | −0.8875 | 4.07 |
| W539 | KRIWQRIK | 12 | 1.71 | −1.525 | 4.28 |
| W540 | KRIWQRWK | 13 | 0.17 | −2.2 | 4.61 |
| W541 | KRWWQRWK | 14 | −1.37 | −2.875 | 4.93 |
| W542 | KRWWQWWK | 15 | −4.03 | −2.425 | 2.78 |
| W543 | RRWWRWWR | 16 | −4.16 | −2.7 | 6.29 |
| W544 | RRWWRWWL | 17 | −5.53 | −1.6625 | 3.81 |
| | | molar | | | |
| | molecular | extinction | molecular | | |
| peptide | formula | coefficient | weight | | |
| W538 | C₄₆H₉₁N₁₈O9 | 0 | 1040.315 | | |
| W539 | C₅₂H₉₂N₁₉O9 | 5550 | 1127.395 | | |
| W540 | C₅₇H₉₂N₂₀O9 | 11100 | 1200.448 | | |
| W541 | C₆₂H₉₂N₂₁O9 | 16650 | 1273.501 | | |
| W542 | C₆₇H₉₁N₁₉O9 | 22200 | 1303.526 | | |
| W543 | C₆₈H₉₅N₂₅O8 | 22200 | 1387.619 | | |
| W544 | C₆₈H₉₄N₂₂O8 | 22200 | 1344.591 | | |

TABLE 5
Antimicrobial activity (MIC, μM) of LL-37 and
its fragments in Roswell Park Memorial Institute (RPMI)
1640 and Dulbecco's Modification of Eagle's
Medium (DMEM) medium against E. coli and S. aureus.

| | S. aureus USA300 | | E. coli E423-17 | |
|---|---|---|---|---|
| Peptides | RPMI | DMEM | RPMI | DMEM |
| LL-37 | ≤4 | ≤4 | ≤4 | 32 |
| KR-12 | 4-8 | 4 | 8 | 2-4 |
| LL-10 | >64 | >64 | >64 | 16-32 |
| KE-10 | >64 | >64 | >64 | >64 |
| LR-10 | >64 | >64 | >64 | >64 |
| RK-9 | >64 | >64 | >64 | >64 |
| RIK-10 | 32 | 64 | 32-64 | 8 |
| KR-8 | >64 | >64 | >64 | 32-64 |

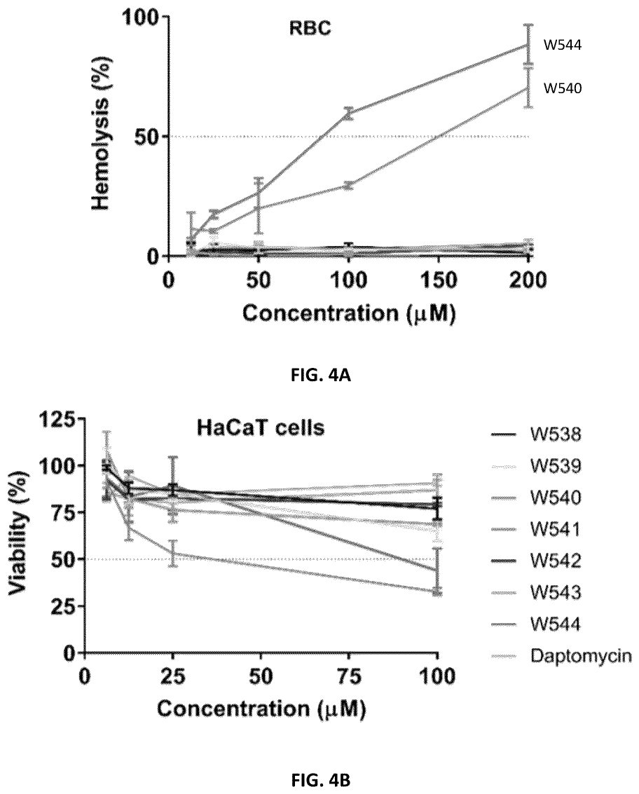

The hemolytic activity of this series of KR-8-derived peptides to human red blood cells was then evaluated (FIG. 4A). Among the seven peptides, only two peptides (W540 and W544) displayed peptide-dose-dependent hemolysis until 200 μM. W544 caused 50% hemolysis (HC₅₀) at about 80 μM, while it took twice the amount of W540 at ˜150 μM to reach 50% hemolysis. Hence, W544 was more hemolytic than W540, consistent with a high hydrophobic content of W544 of 63% (Table 3). Different from W540, peptides W541-W543 were not hemolytic at least until 200 μM, implying that isoleucine at position 3 played an important role in the hemolytic ability of W540 (FIG. 4A). The peptide toxicity to human HaCaT skin cells was also tested (FIG. 4B). Similar to the finding in hemolytic assays (FIG. 4A), W540 and W544 were more toxic than the other peptides (FIG. 4B). W543 was least toxic to HaCaT cells, similar to daptomycin. As a consequence, W543 was selected as a candidate for additional studies. Because W543 is engineered based on the shortest active peptide KR-8 of human LL-37, W543 is referred to hereinafter as LL-37mini, a miniature version of LL-37. LL-37mini is composed of four aromatic Trp amino acids, closely mimicking the four phenylalanine residues of human LL-37 that all bind bacterial anionic lipid (Wang, G. (2008) J. Biol. Chem., 283:32637-32643).

The effects of salts, pH, and human serum on the antibacterial activity of LL-37mini against MRSA in MHB was evaluated. A decrease of pH from 7.2 to 6.3 increased MIC by twofold. The addition of 150 mM NaCl did not alter the MIC. It appeared that both acidic pH and physiological salts had a minimal impact on peptide activity (Table 6). However, 10% human serum reduced the activity of LL-37mini by eightfold. Likewise, serum also influenced the activity of 17BIPHE2. Among the antibiotics tested, only the MIC values of amikacin and muporicin increased fourfold in 10% human serum (Table 6). Multiple human serum proteins can associate with C10-KR8 where KR-8 is conjugated with a C10 fatty acid at the N-terminus (Lakshmaiah, et al. (2021) ACS Infect. Dis., 7:1795-1808). Since serum binding is an inherent property of human LL-37 (Wang, et al. (1998) J. Biol. Chem., 273:33115-33118), all the above peptides retained this property.

TABLE 6
Comparison of antimicrobial robustness of LL-37mini
with antibiotics in MHB. Antimicrobial activity
values are in micromolar. ND: not determined.

| | | | NaCl | 10% | 20% |
|---|---|---|---|---|---|
| Antibiotic | PH 7.2 | pH 6.3 | (150 mM) | serum | serum |
| LL-37 mini | 8 | 16 | 8 | 64 | ND |
| 17BIPHE2 | 4 | 16 | 4 | >64 | ND |
| Linezolid | 8 | 8 | 8 | 8 | 8-16 |
| Amikacin | 8 | 32 | 32 | 32 | 32-64 |
| Gentamicin | 0.5 | 1-2 | 1 | 0.5-1 | 0.5 |
| Tobramycin | 2 | 4 | 4 | 2-4 | 2 |
| Muporicin | 1 | 0.5 | 1 | 4 | 4 |
| Daptomycin | 1 | 1 | 1 | 1 | 1 |
| Nafcillin | 4-8 | 16 | 32 | 16 | 16-32 |
| Vancomycin | 0.5 | 0.5 | 0.5-1 | 0.5 | 0.5-1 |

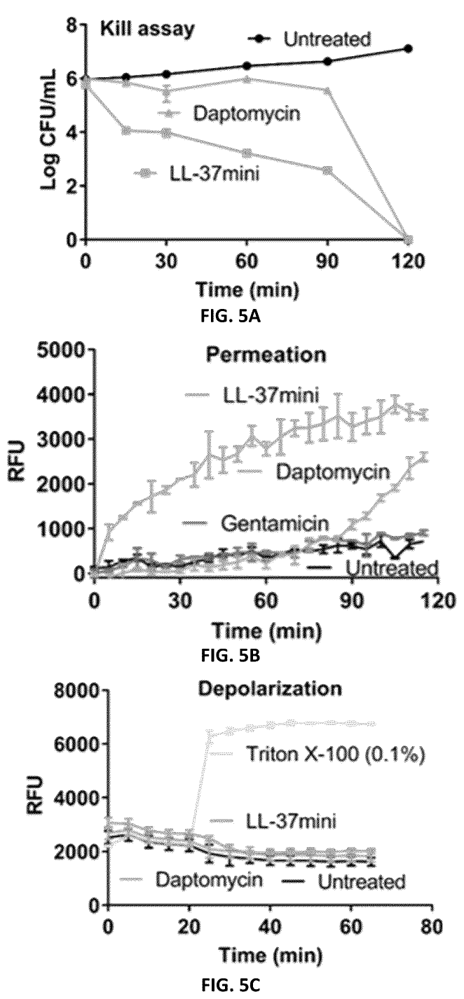

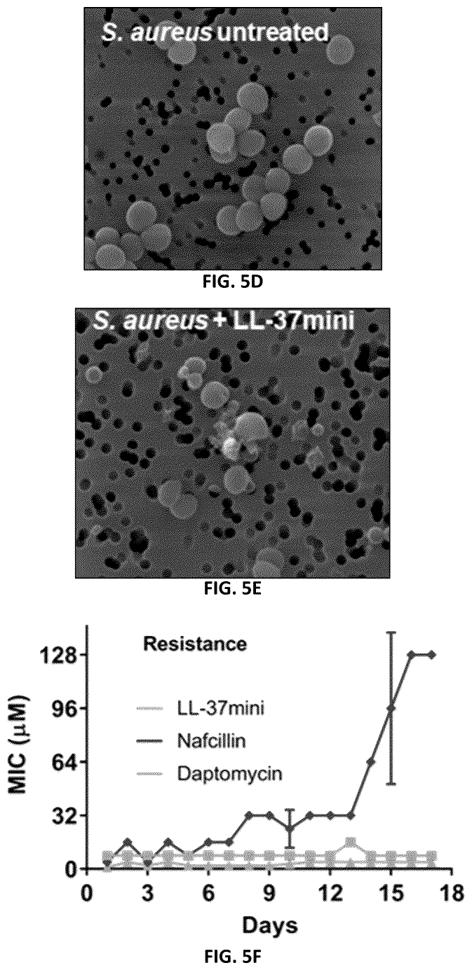

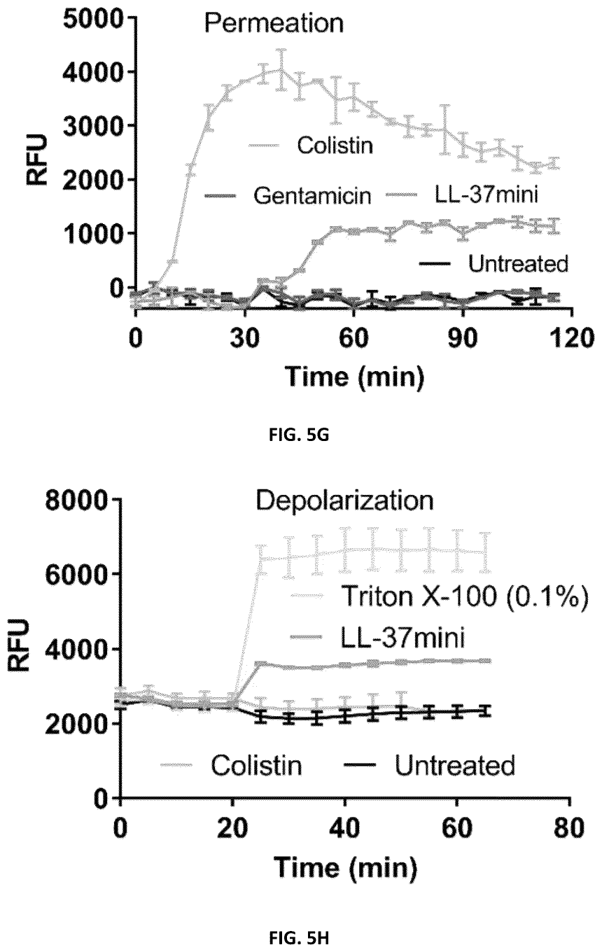

The bacterial killing kinetics of LL-37mini against S. aureus USA300 was also evaluated. While bacterial colony-forming unit (CFU) increased in the untreated culture, it decreased with time and was not detected at 120 minutes due to LL-37mini treatment at 2×MIC (FIG. 5A). Likewise, S. aureus was also killed in 2 hours by daptomycin despite at a slower rate compared to LL-37 mini. LL-37 (FIG. 1) is known to target bacterial membranes (Oren, et al. (1999) Biochem. J., 341(3):501-513; Henzler-Wildman, et al. (2004) Biochemistry 43:8459-8469). To verify this, membrane permeabilization experiments were conducted for LL-37mini in the presence of propidium iodide (PI). In this experiment, one does not anticipate fluorescence increase when S. aureus USA300 was not treated, indicating an intact membrane (FIG. 5B). As a negative control, no fluorescence increase was observed for gentamicin-treated MRSA, either, since this antibiotic inhibits protein synthesis. However, there was a clear fluorescence increase upon treatment with LL-37mini, indicative of membrane damage. As a positive control, daptomycin only showed membrane permeabilization after a delay (Mishra, et al. (2019) Proc. Natl. Acad. Sci., 116:13517-13522). The membrane permeation of E. coli by LL-37 mini was also investigated. Like in the case of MRSA, gentamicin did not permeate the E. coli membrane. However, colistin was a powerful membrane permeator. In the case of LL-37 mini, there appeared to be a 30 minute delay in membrane permeabilization (FIG. 5G). Cationic AMPs may also depolarize bacterial membranes (Lakshmaiah, et al. (2021) ACS Infect. Dis., 7:1795-1808). Triton® X-100 as a positive control showed a strong membrane depolarization of S. aureus USA300. However, LL-37mini did not show a clear effect at the highest concentration that was tested (FIG. 5C). In the case of E. coli, LL-37mini showed some depolarization (FIG. 5H). The cells of S. aureus USA300 were observed with and without peptide treatment using scanning electron microscopy (SEM). While the cells were smooth and spherical without treatment (FIG. 5D), cell damages were evident after treatment with LL-37mini at 2×MIC (FIG. 5E).

It is proposed that membrane damage makes it difficult for bacteria to develop resistance (Lakshmaiah, et al. (2021) ACS Infect. Dis., 7:1795-1808). To verify this, a multiple passage experiment was conducted (FIG. 5F). After 17 days, there was essentially no change in the MIC values of LL-37mini. In the same experiment, MRSA did not develop resistance to daptomycin, either. However, the MIC of nafcillin increased to 128 μM (32-fold), indicating rapid resistance development.

### Antibiofilm Activity of LL-37Mini In Vitro and In Vivo

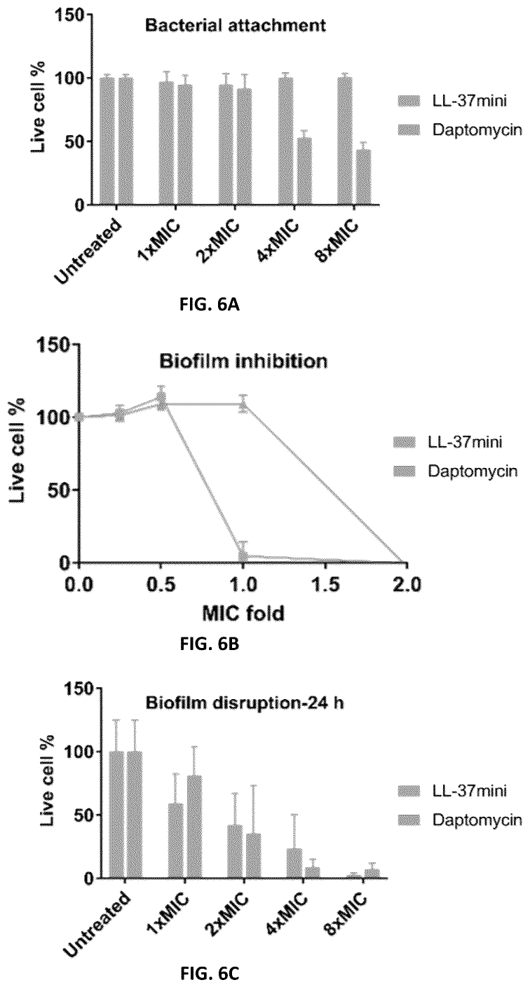

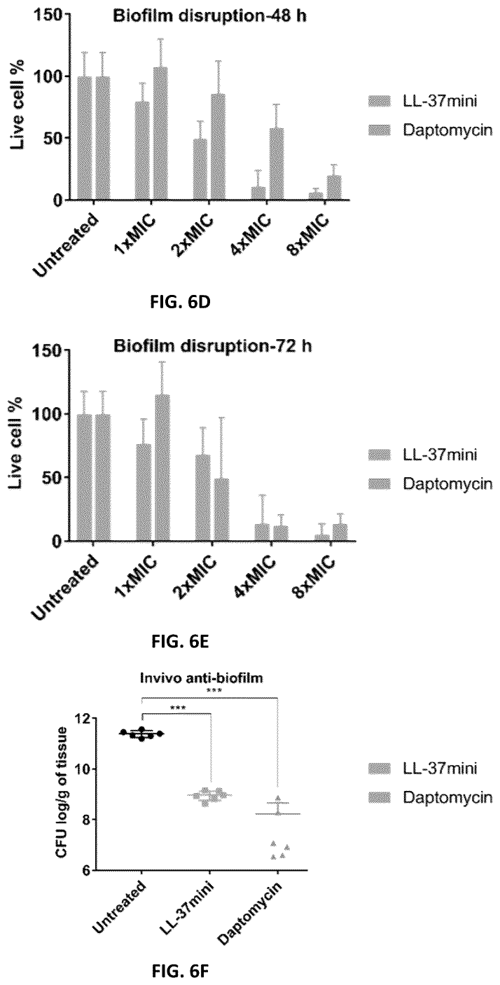

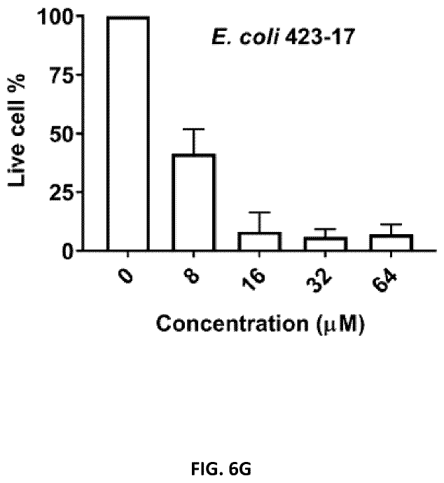

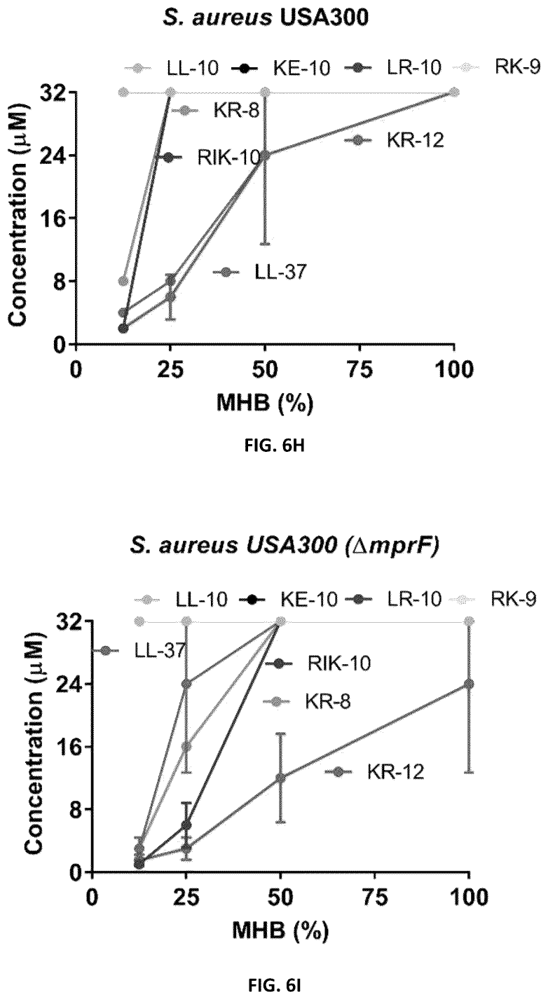

Since biofilms are resistant to antibiotics, the capability of LL-37mini was tested in inhibiting bacterial attachment and biofilm formation and disrupting the preformed biofilms. Surface attachment is the initial step for biofilm formation. In a peptide dose-dependent assay, LL-37mini did not show anti-attachment ability at 4× and 8×MIC. However, daptomycin did (FIG. 6A). In a different experiment, however, LL-37mini was more effective than daptomycin in inhibiting biofilm formation (FIG. 6B). Moreover, a dose-dependent effect was observed against the 24 hours preformed biofilms of S. aureus USA300 (FIG. 6C). At 32 μM, the MRSA biofilms were essentially eliminated by LL-37mini. Better disruptive effects than daptomycin were also observed for LL-37mini against 48 or 72 hour preformed biofilms of S. aureus (FIG. 6D, 6E). The antibiofilm activity of LL-37mini was tested against E. coli E423-17 treated at varying doses. The peptide appeared to be very effective against 24 hour preformed biofilms as it reduced live bacteria by ˜40% when treated at 8 μM (FIG. 6G). At 16 μM, live E. coli cells were almost eliminated. These experiments proved the antibiofilm efficacy of LL-37mini in vitro.

Since LL-37mini is potent and not toxic, its antibiofilm efficacy was tested in vivo using a murine wound model. Wounds were created and infected with S. aureus USA300, and biofilms were allowed to form for 24 hours. Without treatment, the MRSA count reached a CFU below 10¹². This time suffices for biofilm formation (Su, et al. (2019) Mol. Pharmaceutics 16:2011-2020). In vitro, S. aureus can form biofilms in a couple of hours (Decker, et al. (2023) Mol. Pharmaceutics 20:738-749). When the biofilms were treated with daptomycin (10 mg/kg per wound), the bacterial burden was reduced by ˜3 logs. When treated with LL-37mini at 4.4 mg/kg per wound, a bacterial CFU decrease by 2 log was observed (FIG. 6F). Hence, LL-37mini could eliminate MRSA in wound biofilms similar to daptomycin (positive control).

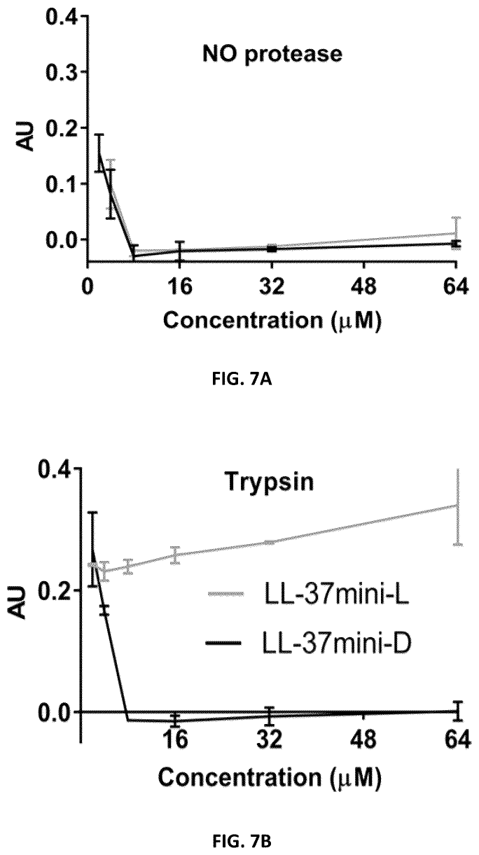

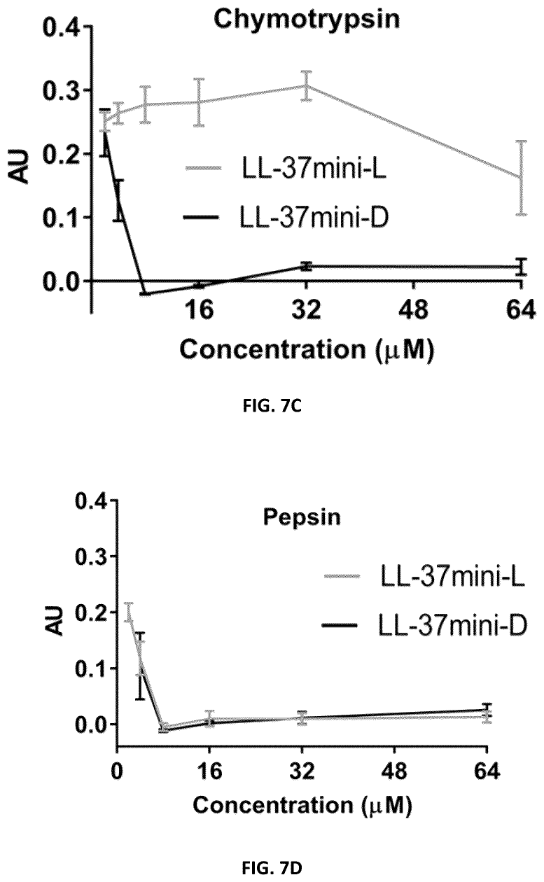

Finally, the antibacterial activity and stability of the above LL-37mini form (L-form) was compared with that made of D-amino acids. The D-form showed the same MIC as the L-form (made of L-amino acids), consistent with membrane targeting (Mishra, et al. (2019) Proc. Natl. Acad. Sci., 116:13517-13522). However, the L-form started to lose activity in the presence of proteases, such as trypsin and chymotrypsin. In contrast, the D-form remained active even after incubation with these two proteases (FIG. 7). Interestingly, both L- and D-forms of LL-37mini retained antibacterial activity against S. aureus USA300 after incubation with pepsin (FIG. 7). It seems that LL-37mini (L-form) is more stable than GF-17 (L-form), which was degraded by pepsin under the same condition (Decker, et al. (2023) Mol. Pharmaceutics 20:738-749).

LL-37 is an important human host defense peptide known to inhibit various pathogens, ranging from viruses and fungi to bacteria (Wang, G. et al. (2019) Advances in Experimental Medicine and Biology; Springer, 2019; Vol. 1117, pp 215-240). Selection of the proper medium is important during antimicrobial assays. This study revealed that the standard MHB medium can be used for antimicrobial screening against E. coli and A. baumannii. This is because the MIC values for E. coli and A. baumannii are least influenced by media dilution (Tables 1 and 2). In rich media, KR-12 was identified as the smallest antibacterial peptide of human cathelicidin LL-37 against E. coli but not S. aureus USA300 (Wang, G. (2008) J. Biol. Chem., 283:32637-32643). Using overlapping synthetic peptides with 12 residues, KR-12 was found to be active (Saporito, et al. (2018) J. Pept. Sci., 24:e3080). This study screened a library of even smaller peptides (8-10 residues) covering the entire length of LL-37 and uncovered the activity of RIK-10 against E. coli in rich media. RIK-10 is the newly identified smallest active peptide of human LL-37 discovered to date in rich media.

However, others frequently failed to observe antimicrobial activity of LL-37 against S. aureus due to the interaction of LL-37 with anionic components in rich media (Turner, et al. (1998) Antimicrob. Agents Chemother., 42:2206-2214; Mishra, et al. (2016) ACS Med. Chem. Lett., 7:117-121). In this study, a diluted MHB for detecting the activity of LL-37 against S. aureus was identified (Table 1). Because this diluted medium is utilized only at the last treatment stage, bacterial culturing and biofilm growth in 100% MHB are as efficient as usual. In 12.5% MHB, LL-37 was found to be as effective against MRSA as the engineered peptide 17BIPHE2. Also, KR-12 was as active as its parent peptide LL-37 (FIG. 3). In addition, the majority of antibiotics retain the same MIC with the dilution of MHB (FIG. 2I). In diluted MHB, an even shorter peptide KR-8 (eight amino acids) is identified as the minimal antibacterial peptide against E. coli (FIG. 2). This medium is also useful for screening activity against S. epidermidis, P. aeruginosa, and K. pneumoniae owing to increased antimicrobial susceptibility (Table 2). Similar screening results were obtained for LL-37 ultrashort peptides in DMEM and RPMI media (Table 5). It is conceivable that the screening sensitivity can be further increased if the antimicrobial assay is conducted using a sensitive bacterial strain such as S. aureus mprF knockout strain (Fey, et al. (2013) mBio 4:e00537-12). The loss of a functional mprF gene disabled the lysine coating on the membrane surface of MRSA (Golla, et al. (2020) ACS Infect. Dis., 6:1866-1881), making the strain more susceptible to killing by cationic peptides such as LL-37 (FIGS. 6H and 6I). Like KR-12 and RIK-10 in rich media, KR-8 retained activity only against Gram-negative E. coli in diluted MHB. It is interesting to note that both KR-8 and RIK-10 are fragments of the core antimicrobial region (FK-16) of LL-37 (FIG. 3). Both ultrashort peptides remain active against E. coli in the DMEM medium used to culture mammalian cells (Table 5). Remarkably, FK-16 is active against both E. coli and S. aureus in rich and diluted MHB. These new results justified the initial selection of the glycine-capped FK-16 template (i.e., GF-17) to engineer 17BIPHE2 into a potent, stable, and selective peptide to eliminate the ESKAPE pathogens (Wang, et al. (2014) ACS Chem. Biol., 9:1997-2002). Taken together, this study demonstrates the sequence-activity relationship of human LL-37 since the two even shorter active peptides KR-8 and RIK-10 discovered herein in a more sensitive medium correspond to the core antimicrobial region (Li, et al. (2006) J. Am. Chem. Soc., 128:5776-5785).

There has been a strong desire to develop human cathelicidin into a new antibiotic. Both library and structure-based design have been utilized (Wang, et al. (2019) Advances in Experimental Medicine and Biology; Springer, Vol. 1117, pp 215-240). It is important to search for the smallest antimicrobial regions from LL-37 to bring down the production cost. Based on the 3D structure of LL-37 (FIG. 1), one would obtain LL-31 if the disordered C-terminus is omitted. Indeed, LL-31 showed potent activity against multiple bacterial pathogens (den Hertog, et al. (2006) Biol. Chem., 387:1495-1502; Wongkaewkhiaw, et al. (2020) PLoS One 15:e0243315). LL-31, corresponding to the long helix in the 3D structure of LL-37 (FIG. 1) (Wang, et al. (2008) J. Biol. Chem., 283:32637-32643), consists of two hydrophobic domains split by serine 9. When the N-terminal hydrophobic domain is removed, SK-24 is obtained (FIG. 1) (Zhang, et al. (2021) Pharmaceuticals 14:1245). Interestingly, this peptide was found to be most helical in a phosphate buffer. However, it is the least toxic antibacterial peptide compared to other LL-37 peptides investigated therein. Based on NMR studies, the major anticancer and antibacterial region, FK-16/GF-17, initially called LL-37 (17-32), was identified (FIG. 1) (Li, et al. (2006) J. Am. Chem. Soc., 128:5776-5785). 17BIPHE2, engineered based on GF-17, is stable, selective, and potent against the ESKAPE pathogens (Wang, et al. (2014) ACS Chem. Biol., 9:1997-2002; Su et al. (2019) Mol. Pharmaceutics 16:2011-2020), Ebola viruses (Yu, et al. (2020) iScience 23:100999), and sperms to avoid unwanted pregnancy (Lee, et al. (2022) Hum. Reprod., 37:2503-2517). KR-12, the smallest antibacterial peptide of LL-37 identified in rich media (FIG. 1) (Wang, G. (2008) J. Biol. Chem., 283:32637-32643), has been used as a template for designing various antimicrobial candidates, ranging from linear to cyclic peptides (Kim, et al. (2017) Eur. J. Med. Chem., 136:428-441; Muhammad, et al. (2023) Biomedicines 11:504). KR-12 has also been lipidated to become lipopeptides (Lakshmaia, et al. (2021) ACS Infect. Dis., 7:1795-1808; Kamysz, et al. (2023) Int. J. Mol. Sci., 24:5505). It was notable that C10-KR8, consisting of KR-8 in conjugation with a capric acid at the N-terminus, was identified as the optimal antimicrobial via screening a lipopeptide library generated from systematically altering both the peptide and fatty acid chain lengths (Lakshmaia, et al. (2021) ACS Infect. Dis., 7:1795-1808). This study has advanced the design of LL-37 by identifying ultrashort active peptide templates such as KR-8 (residues 18-25 of LL-37, FIG. 1) under more sensitive screening condition. Considering the cost advantage of ultrashort peptides, KR-8 was successfully engineered into LL-37mini, a potent, selective LL-37-derived AMP with the shortest length and without conjugation with other molecular moieties. LL-37mini also gains stability to proteases when synthesized using D-amino acids (FIG. 7). The design of LL-37mini was guided by structural knowledge. The deployment of four fused aromatic residues was inspired by the observation that all the four phenylalanine residues of LL-37 directly interdigitate into bacterial membranes to interact with anionic phosphatidylglycerol (Wang, G. (2008) J. Biol. Chem., 283:32637-32643). LL-37mini has the desired activity spectrum against both S. aureus and P. aeruginosa (Table 3), two major pathogens in chronic wounds. The antimicrobial and wound-healing properties of LL-37mini can be incorporated into nanofibers. When formulated, even the L-form of LL-37mini will work well (Decker, et al. (2023) Mol. Pharmaceutics 20:738-749). Collectively, this study has expanded the knowledge of the structure-activity relationships of human LL-37 and enriched the reservoir of LL37-derived antimicrobials to combat drug-resistant pathogens.

A caveat in antimicrobial screening is the failure to detect active candidates. This study has identified a diluted MHB medium that enabled the observation of antibacterial and antibiofilm activity of human cathelicidin LL-37 against S. aureus USA300. By screening a small library of ultrashort peptides in 12.5% MHB, two active peptides (KR-8 and RIK-10) within LL-37 were identified. Notably, KR-8 and RIK-10 correspond to the core antimicrobial region of LL-37 initially discovered from NMR structural studies (Li, et al. (2006) J. Am. Chem. Soc., 128:5776-5785). Shorter peptides are desired for antimicrobial development due to reduced production cost. The antimicrobial activity of the KR-8 template was enhanced via peptide engineering. The engineered peptide LL-37mini is both potent and selective. It also showed antibiofilm efficacy in vitro and in vivo. LL-37mini constitutes a novel lead for developing new antimicrobial and antibiofilm agents. The diluted medium obtained here is useful for initial antimicrobial screen to identify antimicrobials from natural sources or artificial libraries. Antibacterial assays in such a medium may increase positive hits against numerous bacteria, including S. aureus, S. epidermidis, P. aeruginosa, and K. pneumoniae.

### Example 2

Antibiotic resistance constitutes a serious concern as it may put us back to the pre-antibiotic age. In particular, Gram-negative pathogens are difficult to eliminate due to the double membrane structure. It is estimated that 10 million people could die by 2050 (O'Neill, J. (2016) Tackling Drug-Resistant Infections Globally: Final Report and Recommendations. Review on Antimicrobial Resistance. HM Government, pages 1-84). While a comprehensive strategy, ranging from controlled use of existing antibiotics to education of the public, is required, the search of potent antimicrobials is always an option.

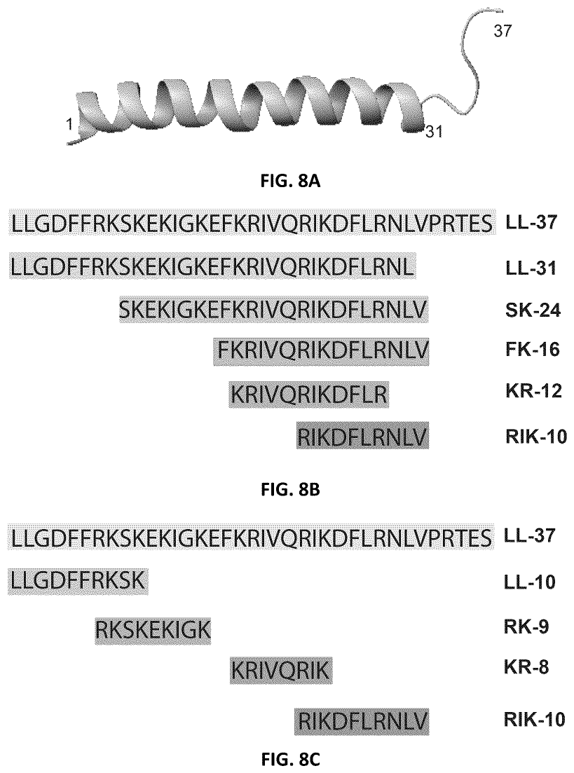

Most of the current drugs are inspired by or derived from nature. Antimicrobial peptides (AMPs) are natural compounds that play an essential role in a variety of organisms, ranging from plants to animals (Eliopoulos, et al. (1986) Antimicrob. Agents Chemother., 30(4):532-535; Schittek, et al. (2001) Nat. Immunol., 2(12):1133-1137; Wang, G. (2014) Pharmaceuticals 7: 545-594). As of Apr. 5, 2024, the antimicrobial peptide database (APD) documented 3215 natural AMPs with 20 reported in 2024 (Wang, G. (2020) Protein Sci., 29:8-18; Wang, et al. (2016) Nucleic Acids Res., 44(D1):1087-1093; Wang, G. (2023) Protein Sci., 32(10): e4778). Among them, 400 originated from bacteria, 257 from plants, and 2508 from animals. Of the 148 human AMPs, the major classes are defensins, cathelicidin, ribonucleases, dermcidin, histatins, and antimicrobial cytokines (Wang, G. (2014) Pharmaceuticals 7: 545-594). Most of these peptides are cationic, but dermcidin has a net charge of −2 (Schittek, et al. (2001) Nat. Immunol., 2(12):1133-1137). While defensins are known to form a β-sheet structure stabilized by three pairs of disulfide bonds, such a bond is absent in human cathelicidin, which forms an α-helical structure with the C-terminal tail disordered (FIG. 8A) (Johansson, et al. (1998) J. Biol. Chem., 273(6):3718-24; Wang, G, (2008) J. Biol. Chem., 283(47):32637-32643). The most common mature human cathelicidin peptide, LL-37, are able to eliminate viruses, bacteria, fungi, and parasites (Xhindoli, et al. (2016) Biochim. Biophys. Acta 1858(3): 546-566; Wang, et al. (2019) Adv. Exper. Med. Biol., 1117:215-240). Significantly, it can inhibit SARS-CoV-2, Zika, and Ebola viruses (Wang, et al. (2021) ACS Infect. Dis., 7(6):1545-1554; He, et al. (2018) Front. Immunol., 9:722; Ripperda, et al. (2022) Pharmaceuticals 15(5):521). While conventional antibiotics are not effective against multi-drug resistant bacteria and biofilms, LL-37 remains potent (Overhage, et al. (2008) Infect. Immun., 76(9):4176-4182; Mishra, et al. (2016) ACS Med. Chem. Lett., 77(1):117-121). In addition, human LL-37 plays a role in chemotaxis, lipopolysaccharide (endotoxin) neutralization, and wound healing (Duplantier, et al. (2013) Front. Immunol., 4:143; Scott, et al. (2002) J. Immun., 169(7):3883-3891). The wide-spectrum activity of LL-37 has stimulated a high interest in developing it into novel antimicrobials. To reduce the cost for chemical synthesis, both structural biology and library approaches have been utilized to map the active regions (Braff, et al. (2005) J. Immun., 174(7):4271-4278; Molhoek, et al. (2009) Biol. Chem., 390(4):295-303; Nagant, et al. (2012) Antimicrob. Agents Chemother., 56(11):5698-5708; Li, et al. (2006) J. Am. Chem. Soc., 128(17):5776-5785; De Breij, et al. (2018) Sci. Transl. Med., 10(423):eaan4044). Based on NMR structural studies of LL-37 fragments, the major antimicrobial region corresponding to residues 17-32 (GF-17/FK-16) was identified as well as the antimicrobial core peptide LL-37(17-29) (i.e., FK-13) (Li, et al. (2006) J. Am. Chem. Soc., 128(17): 5776-5785). GF-17 was successfully converted to a selective, potent, and stable peptide to eliminate the ESKAPE pathogens (Wang, et al. (2014) ACS Chem. Biol., 9(9):1997-2002). Subsequent synthesis of additional short peptides led to the discovery of the first minimal antibacterial peptide KR-12 (Wang, G. (2008) J. Biol. Chem., 283(47):32637-32643). This peptide template has been utilized to engineer both linear and cyclic peptides, including lipopeptides (Kim, et al. (2017) Eur. J. Med. Chem., 136:428-441; Muhammad, et al. (2023) Biomedicines 11(2):504; Kamysz, et al. (2023) Int. J. Mol. Sci., 24(6): 5505; Lakshmaiah, et al. (2021) ACS Infect Dis 77 (6):1795-1808). The study here of ultrashort peptides (≤10 amino acids) led to the discovery of another small antibacterial peptide (RIK-10) that inhibits E. coli but not MRSA. The major LL-37 peptides derived from the structural studies are depicted in FIG. 8B. LL-31 corresponds exactly to the helical region of LL-37, while SK-24 corresponds to the helical region without the N-terminal hydrophobic segment (residues 1-8) separated by the hydrophilic S9 on the hydrophobic surface of LL-37 (Wang, G. (2008) J. Biol. Chem., 283(47):32637-32643).

This study took a different avenue to peptide discovery. Since small peptides are cost effective to make, it was hypothesized that potent and selective peptides could be designed by directly cutting the helical region of LL-37 (FIG. 8A) into four small overlapping ultrashort segments (≤10 amino acids) (FIG. 8C). Before the design, three of these natural segments, LL-10, RK-9, and KR-8, are not antibacterial and only RIK-10 shows a moderate activity in standard MHB. The activity of all these segments was enhanced via knowledge-based design. The knowledge-based design was conducted based on the statistical differences between natural and synthetic peptides. Due to optimization, synthetic peptides tend to have higher basic amino acids and hydrophobic amino acids (Wang, G. (2023) Protein Sci., 32(10):e4778). The bulky aromatic tryptophan is especially powerful in membrane binding and conferring activity to short peptides (Hilpert, et al. (2005) Nat. Biotechnol., 23(8):1008-12; Schibli, et al. (2002) Biochem. Cell Biol., 80(5):667-77; Mishra, et al. (2017) Acta Biomater., 49:316-328). Antibacterial and antibiofilm activity of the designed peptides were tested both in vitro and in vivo. The mechanism of action of these peptides was also investigated and the three-dimensional structure of the most potent peptide was determined. The similar activity against Gram-positive bacteria but different activity against Gram-negative pathogens allowed for the determination that the distribution of cationic amino acids along the sequence plays a role because these peptides possess similar net charge and hydrophobic ratios. To view the charge distribution, charge density plots for these designed peptides were generated. The cationic amino acids of three peptides with good activity against Gram-negative bacteria were found mostly at the N-termini. Next, this finding was validated by making sequence reversed peptides. Finally, the antimicrobial peptide database was searched and numerous anti-Gram-negative peptides were identified with such a charge distribution in the sequences, thereby uncovering a useful strategy for designing effective AMPs to combat Gram-negative pathogens.

### Materials and Methods

### Chemicals and Peptides

All the chemicals were purchased from established vendors such as Fisher and Sigma. Peptides were made by Genemed Synthesis, Inc. (San Antonio, TX). All the peptides were >95% pure by HPLC. The correct mass of each peptide was validated by Mass Spectrometry. Peptides were quantitated by measuring UV absorbances at 280 nm (with W) or at 215 and 225 nm (without W) based on the Waddell's method (Waddell, W. J. (1956) J. Lab. Clin. Med., 48(2):311-314).

### Antibacterial Assay

Peptide activity against bacteria were tested using the CLSI standard procedure with minor modifications (Clinical and Laboratory Standards Institute (2018) Performance standards for antimicrobial susceptibility testing, 9th ed.; Mishra, et al. (2019) Proc. Natl. Acad. Sci., 116(27):13517-13522). In brief, a peptide concentration gradient with twofold dilution was made in the 96-well polystyrene microplate at 10 μL per well. Bacteria were grown to the exponential phase (i.e., optical density at 600 nm≈0.5), diluted to ˜10⁵ CFU/mL in MHB, and partitioned into the 96-well microplate at 90 μL per well. The microplates were incubated at 37° C. overnight and read on a ChroMate® 4300 Microplate Reader at 600 nm (GMI, Ramsey, MN). The peptide concentration in the wells without bacterial growth is the minimal inhibitory concentration (MIC). As the convention, MIC values were represented as ranges.

For pH effects on peptide activity, the pH of the medium was adjusted to a targeted pH for autoclave and then remeasured at room temperature. For salt and serum effects on peptide activity, stock NaCl solution or human serum were added to the media.

### Killing Kinetics

For bacterial killing kinetics study, MRSA USA300 and P. aeruginosa E411-17 was grown to the exponential phase (OD₆₀₀˜0.3) in MHB. The culture was diluted to OD₆₀₀ 0.001 (˜10⁵ CFU/mL). One milliliter of bacterial suspension was then mixed with the peptide, colistin or daptomycin at 4×MIC and incubated at 37° C. After 15, 30, 60, 90, and 120 minutes incubation, 50 μL was taken and serially diluted with 1×PBS. Then, 50 μL of the diluted suspension was plated on Mannitol Salt Agar (MRSA USA300) or Cetrimide Agar (P. aeruginosa E411-17) plates (NEOGEN, MI). The plates were incubated overnight at 37° C. for bacterial CFU count the next day.

### Hemolytic Assay

The hemolytic effects of peptides were evaluated (Narayana, et al. (2020) Proc. Natl. Acad. Sci., 117(32):19446-9454). In brief, human red blood cells (UNMC Blood Bank) were washed (3×) and diluted to 2% using the saline solution. Aliquots of 90 μL were mixed with 10 μL of serially diluted peptide solutions. After incubation at 37° C. for 1 hour, plates were centrifuged at 500 g for 5 minutes and aliquots of the supernatant were transferred to a new 96-well microplate to measure the amount of hemoglobin released at 545 nm (relative to 1% Triton® X-100) using a ChroMate® Microplate Reader.

### Cytotoxicity Assay

Human keratinocytes HaCaT cells were grown in Dulbecco's Modification of Eagles's Medium/High Glucose (DMEM, Hyclone, UT) containing 10% fetal bovine serum (FBS) (Mediatech, Corning, Manassas, VA). HepG2 cells were cultured in ATCC-formulated Eagle's Minimum Essential Medium with 10% FBS. A549 cells were grown in ATCC-formulated F-12K Medium containing 10% FBS. Cells were grown at 37° C. in 5% CO₂, and the medium was changed every other day. Peptides were diluted in a 96-well polystyrene microplate (10 μL each well) and mixed with 90 μL of cell suspensions (10⁵ cells/mL) in their respective media. The mixture was incubated at 37° C. in a 5% CO₂ for 24 hours. Then, 20 μL of MTS reagent (MTS cell proliferation assay kit, Promega, WI) was added to each well and incubated at 37° C. for 2 hours. The absorbance was determined at 492 nm using a microplate reader. The percentage viability was determined using the following formula: Percentage viability=(OD value of treated cells/OD value of untreated cells)×100, where OD is the optical density.

### Bacterial Membrane Permeabilization

Bacterial membrane permeation was conducted (Yeaman, et al. (1998) J. Clin. Invest., 101(1):178-87). In brief, the peptide ladder was prepared as described for the antibacterial assay with 10 μL in a black COSTAR 96-well plate. Propidium iodide (PI) (MP Biomedicals, Solon, OH) was prepared in the dark and dissolved in DMSO (Thermo Fisher Scientific, NY) to 20 mM. This PI stock solution was further diluted to 1 mM with water and 2 μL of 1 mM PI was added to each well. Exponential phase MRSA USA300 or P. aeruginosa E411-17 was diluted to OD₆₀₀ 0.11 with tryptic soy broth (TSB) and 88 μL was added to each well. The plate was incubated at 37° C. with continuous shaking in a FLUOstar® Omega (BMG LABTECH, NC) microplate reader. The sample was read every 5 minutes for 24 cycles with excitation and emission wavelengths at 584 nm and 620 nm, respectively.

### Bacterial Membrane Depolarization

Bacterial membrane depolarization was conducted (Marks, et al. (2013) PLoS One 8(5):e63158). Briefly, an overnight culture of MRSA USA300 or P. aeruginosa E411-17 was inoculated into TSB and grown to exponential phase. Bacteria were washed with 1×PBS, re-suspended in twice the volume of 1×PBS containing 25 mM glucose, and incubated at 37° C. for 15 minutes. Then, 500 nM (final concentration) of the DiBAC4 (3) bis-(1, 3-dibutylbarbituric acid) trimethine oxonol (ANASPEC, CA) was added and vortexed gently. Aliquots of 90 μL of the energized bacteria solution were loaded to the 96 well plates (Corning COSTAR, AZ) and placed in a FLUOstar® Omega microplate reader (BMG LABTECH, NC). Fluorescence was read for 20 minutes at excitation and emission wavelengths of 485 nm and 520 nm, respectively. Then, 10 μL of peptide solutions was added and fluorescence readings were recorded for 40 minutes. Triton® X-100 (0.1%) was used as a positive control.

In vitro Antibiofilm Activity

### Inhibition of Bacterial Attachment

Bacterial attachment was conducted (Mishra, et al. (2016) ACS Med. Chem. Lett., 77(1):117-121; Duplantier, et al. (2013) Front. Immunol., 4: 143). In brief, an overnight culture of MRSA USA300 or P. aeruginosa E411-17 was grown overnight in TSB media to an optical density (OD₆₀₀) of ˜1.0. Then, 180 μL of this culture were added to each well of the microtiter plates containing 20 μL of various MIC folds (1×, 2×, 4×, and 8×MIC) of peptides or antibiotics. The plates were then incubated at 37° C. for 1 hour. Next, media was aspirated, washed with 1×PBS, and 200 μL of TSB containing XTT 10% [2, 3-bis(2-methyloxy-4-nitro-5-sulfophenyl)-2H-tertazolium-5-carboxanilide](ATCC, VA) solution was added. After incubation at 37° C. for 2 hours, absorbance was read at 450 nm using a ChroMate® microtiter plate reader. TSB containing 10% XTT served as blank, while bacterial culture treated with water served as a positive control. Percentages of viable cells in biofilms were plotted by assuming 100% growth in water treated control.

### Effects of Peptides on Established Biofilms in Vitro

Bacteria (S. aureus USA300 LAC or P. aeruginosa E411-17) were grown up in TSB media overnight. A second inoculation was made the next day to reach an exponential phase (OD₆₀₀˜0.4). Microtiter plates (96 wells, Corning Costar Cat No. 3595), after aliquoting with 180 μL of the culture to each well, were incubated at 37° C. for 24 hours to form biofilms in rich TSB. Media were aspirated post incubation and the attached biofilms were washed with 1×PBS to remove the planktonic bacteria. Each well was aliquoted with 20 μL of 10× peptide solution and 180 μL of 10% fresh MHB, and plates were further incubated at 37° C. for 24 hours. Biofilms treated with water served as the positive control while media without bacterial inoculation served as the negative control. Live cells in the biofilms were quantitated using the XTT as described above. Absorbance was read at 450 nm (only media with XTT containing wells served as the blank) using a ChroMate® microtiter plate reader. Percentage biofilm growth (the plot legend is antibiofilm activity) for the peptides was plotted by assuming 100% biofilm growth in bacterial control alone. The data were represented as mean±SD, plots were generated using GraphPad prism 7, where * indicates p<0.05, ** p<0.01, and ***P<0.001, and ****p<0.0001 (one-way analyses of variance).

### In Vivo Efficacy of the Designed Peptides

### Preparation of Inoculum

An overnight culture of S. aureus USA300 LAC was inoculated in fresh MHB and incubated at 37° C. for 3 hours. The suspension was centrifuged at 5,000 g for 5 minutes and the supernatant was discarded. The bacteria were re-suspended in 1×PBS and the OD₆₀₀ was adjusted to 0.6 (˜3×10⁷ CFU/mL).

### Experimental Animals

Female BALB/c mice (3-4 weeks, ˜20 g) were fed with standardized food (Teklad Laboratory diet for rodents) and water (Hydropac® Alternative Watering System) ad libitum. Mice were kept in ventilated cages (IVCs) at a temperature of 20-24° C., humidity of 50-60%, 60 air exchanges per hour, and a 12/12-hour light/dark cycle. All materials, including IVCs, lids, feeders, bottles, bedding, and water, were autoclaved before use. All animal manipulations were performed in a class II laminar flow biological safety cabinet. The study was approved by the Institutional Animal Care and Use Committee (IACUC) of UNMC (Protocol no. 22-015-08-FC).

### In Vivo Biofilm Assay

Mice were anesthetized by intraperitoneal injection of ketamine-xylazine (100 mg/kg+10 mg/kg). The dorsal back hair was removed with a clipper and depilatory cream, followed by cleaning the shaved area with isopropyl alcohol (70%) and povidone iodine swabs. Two full-thickness skin wounds with a diameter of 6 mm were then created using a disposable biopsy punch (Integra Miltex, MA). The wounds were immediately inoculated with MRSA USA300 (10 μL of ˜3×10⁷ CFU/mL) and wounds were covered with a transparent film dressing (3M Tegaderm™, Deutschland GmbH, Neuss, Germany). Buprenorphine (0.05 mg/kg, SC) was injected and mice were kept in individual cages. After 24 hours of inoculation and biofilm formation, treatment groups received 3×10 μL each peptide or daptomycin dissolved in sterile water. Treatment was applied topically on the wound and wounds were covered with the transparent film dressing. The infected control group received an equal volume of sterile water. After 24 hours from the time of treatment, mice were euthanized under CO₂ and specimens were collected using sterile 8 mm biopsy punch (Integra Miltex, MA) into sterilized tubes containing 2 mL of PBS. Tissues were blended using a homogenizer, diluted further in 1×PBS and plated on mannitol salt agar plates. Finally, the plates were incubated at 37° C. for 20 hours and the CFUs were counted.

### Structural Determination of RIK-10+ by NMR Spectroscopy

NMR data were collected for RIK-10+ (>95% pure) in complex with deuterated DPCd38 (1:60 molar ratio) in 300 μl 90% H₂O/10% D₂O on the 600 MHz Bruker spectrometer at 25° C. and pH 4.5 (Narayana, et al. (2020) Proc. Natl. Acad. Sci., 117(32):19446-9454). The NMR data were processed using NMRpipe (Delaglio, et al. (1995) J. Biomol. NMR 6(3):277-293) and signals were assigned via Pipp (Garrett, et al. (1991) J. Magn. Reson., 95:214-220). Structures were then determined using Xplor-NIH (Schwieters, C D, et al. (2018) Protein Sci., 27(1):26-40). Structural quality was checked using Procheck (Laskowski, et al. (1996) J. Biomol. NMR 8(4):477-86). Structural images were made using MOLMOL (Koradi et al. (1996) J. Mol. Graph., 14(1):29-32).

### Charge and Hydrophobic Density Plots for Antimicrobial Peptides

Charge and hydrophobic densities for each amino acid in helical peptides were calculated by writing an in-house R program. The window size was four based on the 3.6 amino acids per turn in an α-helix. The calculations started from the N-terminus and ended at a residue whose fourth count is the C-terminal residue. Multiple sequences could be handled in one shot. Figures were made based on the calculated values using GraphPad Prism 7.

### Statistical Analysis

Bacterial minimal inhibitory concentrations of peptides were expressed as ranges. Other data were analyzed using GraphPad Prism 7 (GraphPad Software, Inc., San Diego, CA) and measured values were expressed as mean±standard deviation. One-way analyses of variance (ANOVA) were used to compare the mean values among treatment groups. P<0.05 was considered statistically significant.

### Results

### Peptide Design

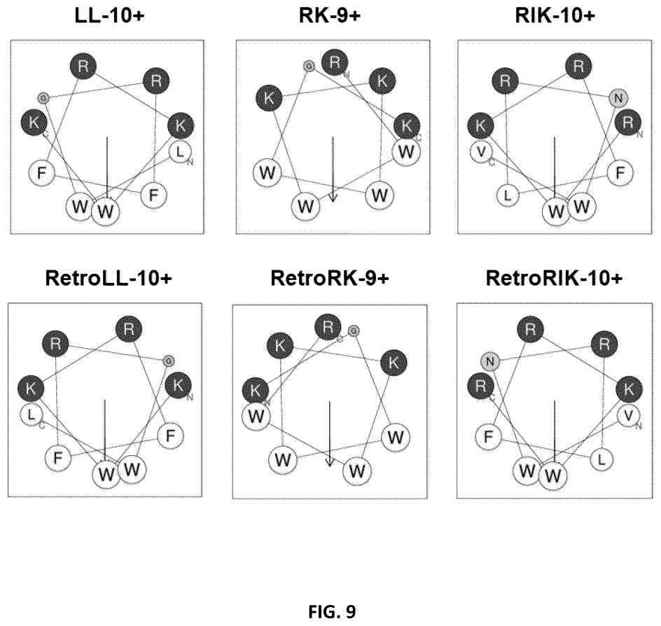

Four peptide segments, including LL-10, RK-9, KR-8, and RIK-10 (FIG. 8C), were produced based on the long helical region (FIG. 8A) of human LL-37 determined by 3D triple-resonance NMR spectroscopy (Wang, G. (2008) J. Biol. Che., 283(47):32637-32643). While LL-10, RK-9, and KR-8 are not antibacterial in the standard MHI medium, RIK-10 is active against E. coli but not S. aureus USA300. To enhance the activity of these peptides, three- to five amino acid substitutions in each segment were made. Based on the preferred amino acids in amphipathic peptides (Wang, G. (2023) Protein Sci., 32 (10):e4778), the following changes were made: (1) in LL-10, L2 was changed to W, and D4 was altered to R, and S9 was replaced with tryptophan (W); (2) four W residues were deployed in both RK-9+ and KR-8+ to increase peptide activity; and (3) in RIK-10, D4 was substituted with R and changed both 12 and L9 to W. The peptide sequences before change are shown in FIG. 8C, while their enhanced sequences (+) are listed in Table 7, which also include physiochemical properties. As these amino acid changes are conservative, the new peptides have the potential to retain the helical structure as observed for the parent peptide. After these changes the four designer peptides are comparable in length (8-10 amino acids), hydrophobic ratios (Pho: 44-50%), and net charges (+3 to +4). The hydrophobic ratios and net charges in these small peptides are similar to those in the majority of natural AMPs in the APD (aps.unmc.edu) (Wang, et al. (2016) Nucleic Acids Res., 44(D1):1087-1093; Wang, G. (2022) Methods in Enzymology 663:1-18). However, there are some variations in GRAVY (Kyte, et al. (1982) J. Mol. Biol., 157(1):105-132) and Boman index (Boman, H. G. (2003) J. Intern. Med. 254:197-215) in Table 7. The GRAVY values of the two Trp-rich peptides, RK-9+ and KR-8+, are twice as negative as those of LL-10+ and RIK-10+ with comparable values (˜−1). While three of the designed peptides have a Boman index in the range of 2.36 to 2.78, RIK-10+ has the highest value at 4.03 kcal/mol. Peptide hydrophobicity and hydrophobic moment was also calculated by assuming an α-helical structure (see the helical wheel plots in FIG. 9) (Eisenberg, et al. (1984) J. Mol. Biol., 179(1):125-42; Gautier, et al. (2008) Bioinformatics 24(18):2101-2). RIK-10+ is slightly less hydrophobic but with a higher hydrophobic moment (Table 7).

TABLE 7
Amino acid sequences and properties of the four peptides designed based on
the helical region of human LL-37. Changed amino acids are in bold. Calculated
using the APD website: aps.unmc.edu/prediction. Pho is the hydrophobic ratio
obtained by summing the eight hydrophobic amino acids (A, V, I, L, M, F, W, and C)
divided by peptide length. Hydrophobicity (Pho2) and hydrophobic moment
(Eisenberg, et al. (1984) J. Mol. Biol., 179(1): 125-42) were calculated using
HeliQuest (heliquest.ipmc.cnrs.fr/cgi-bin/ComputParams.py) by selecting alpha-helix
and full window (Gautier, et al. (2008) Bioinformatics 24(18):2101-2). NA: The
HELIQUEST program was unable to do calculations for KR-8+ due to a shorter
sequence.

| | | SEQ ID | | | Net | |
|---|---|---|---|---|---|---|
| Peptide | Sequence | NO | Length | Pho | charge | GRAVY |
| LL-10+ | LWGRFFRKWK | 30 | 10 | 50 | +4 | −0.96 |
| RK-9+ | RWWKKWWGK | 31 | 9 | 44 | +4 | −2.24 |
| KR-8+ | KRWWQWWK | 32 | 8 | 50 | +3 | −2.43 |
| RIK-10+ | RWKRFLRNWV | 33 | 10 | 50 | +4 | −1.19 |

| | Boman index | Pho2 | Moment |
|---|---|---|---|
| LL-10+ | 2.44 | 0.578 | 0.899 |
| RK-9+ | 2.36 | 0.558 | 0.866 |
| KR-8+ | 2.78 | NA | NA |
| RIK-10+ | 4.03 | 0.459 | 0.915 |

### Antimicrobial Activity of the Designed Peptides

The antimicrobial activity of the four designed peptides (Table 8) was then measured using the standard microdilution assay in Mueller Hinton Broth (MHB). All these peptides are highly purified with a HPLC purity greater than 95%. Two Gram-positive (S. aureus and S. epidermidis) and three Gram-negative bacterial strains (P. aeruginosa, E. coli, and A. baumannii) were tested. Most of the Gram-negative bacteria contain extended-spectrum beta-lactamase (ESBL) and are resistant to beta-lactam antibiotics. LL-10+ and RK-9+ showed moderate activity (MIC 8-32 μM) against four of these bacteria except for A. baumannii (MIC>32 μM). While KR-8+ and RIK-10+ were moderately active against Gram-positive Staphylococcal strains, including methicillin-resistant S. aureus (MRSA), they displayed excellent activity against Gram-negative pathogens, especially E. coli and P. aeruginosa (MIC 4-8 μM). In addition, RIK-10+ was more active against A. baumannii than the other three peptides (Table 8). It appeared that the activity of these peptides against Gram-negative pathogens is in the following order: RIK-10+>KR-8+>RK-9+>LL-10+. Since these peptides are more active against Gram-negative bacteria than Gram-positive bacteria, they are highly desired considering a general lack of new drugs against Gram-negative pathogens. Hence, unique peptides (e.g., KR-8+ and RIK-10+) were designed which differ from the designed LL-37 peptides such as C10-KR8 and 17BIPHE2 with broad antibacterial activity against both Gram-positive and Gram-negative pathogens. As positive controls, daptomycin and colistin displayed anticipated activity spectra against Gram-positive and Gram-negative bacteria only, respectively.

TABLE 8
Antimicrobial activity of human LL-37 derived
antimicrobial peptides. ND: activity not
detected at the highest concentration tested.

| | Gram-positive bacteria | | Gram-negative bacteria | | |
|---|---|---|---|---|---|
| | | S. | P. | | A. |
| | S. aureus | epidermidis | aeruginosa | E. coli | baumannii |
| Peptide | USA300 | 1457 | E411-17 | E423-17 | B28-16 |
| LL-37 | >32 | >32 | 16 | 8 | 8 |
| LL-10+ | 16 | 32 | 8-16 | 32 | >32 |
| RK-9+ | 8-16 | 16 | 8-16 | 8 | >32 |
| KR-8+ | 8-16 | 16-32 | 8 | 4 | 16-32 |
| RIK-10+ | 16 | >32 | 4 | 4 | 8 |
| C10-KR8 | 4 | 4 | 8 | 8 | 4 |
| 17BIPHE2 | 4 | 8 | 4 | 4 | 4 |
| Colistin | ND | ND | 1-2 | 1-2 | 2 |
| Daptomycin | 0.5-1 | 0.5-1 | ND | ND | ND |

Peptide activity was also further increased by substituting Trp with biphenylalanine (Bip). Bip-KR-8+ was obtained by altering both W4 and W7 of KR-8+ to Bip, while Bip-LL-10+ was designed by substituting W9 of LL-10+ to Bip. In Bip-RIK-10+, both W2 and W9 of RIK-10+ were replaced by Bip. Such a Trp to Bip change increased peptide activity against Staphylococcal strains (MIC 1-4 μM). In particular, Bip-RIK-10+ was potent against both Gram-positive and negative pathogens, including K. pneumoniae E406-17, which was not killed by RIK-10+(Table 9).

TABLE 9
Minimal inhibitory concentration (MIC, μM) and 50%
hemolytic concentration (HC₅₀, μM) of additional designed peptides. HC₅₀, the peptide concentration that caused 50%
hemolysis; HC₁₀, the peptide concentration that caused 10%
hemolysis. Hence, the latter is an important indicator for
peptide toxicity as well. The table also provides the therapeutic
index (HC₁₀/MIC) based on HC₁₀ and MIC against E. coli.

| | Bip-KR-8+ | Bip-LL-10+ | Bip-RIK-10+ |
|---|---|---|---|
| S. aureus USA300 | 4 | 2-4 | 1 |
| S. epidermidis 1457 | 4 | 4 | 2 |
| E. coli E416-17 | 16 | 4 | 2 |
| P. aeruginosa E411-17 | 16-32 | 4 | 2 |
| K. pneumoniae E406-17 | >32 | >32 | 2-4 |
| A. baumannii B28-16 | 32 | 8 | 2-4 |
| Hemolysis (HC50) | >200 | >200 | ~30 |
| Hemolysis (HC10) | 15 | 100 | < 8 |
| HC₁₀-based therapeutic | ~1 | 25 | <4 |
| index | | | |

### Bacterial Growth Inhibition and Killing Kinetics

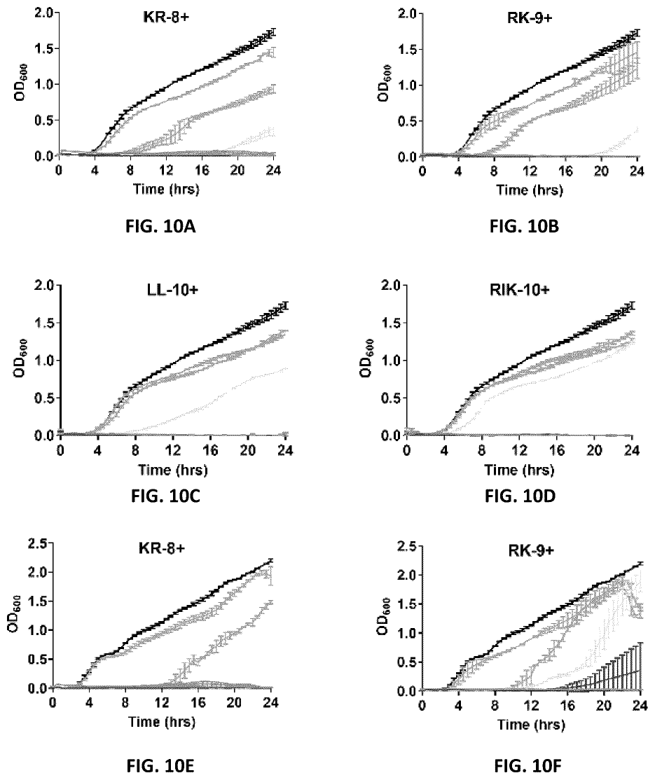

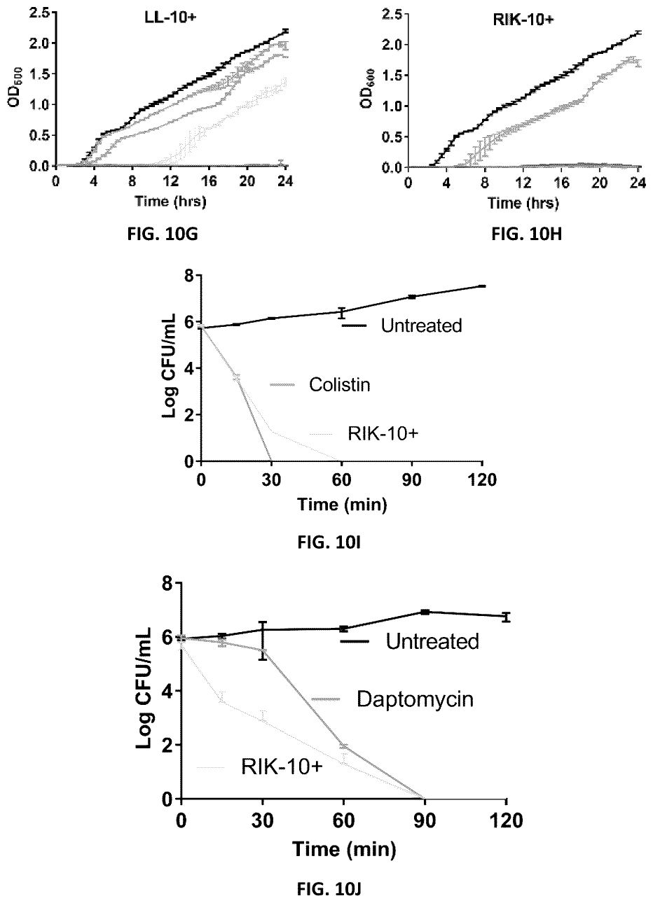

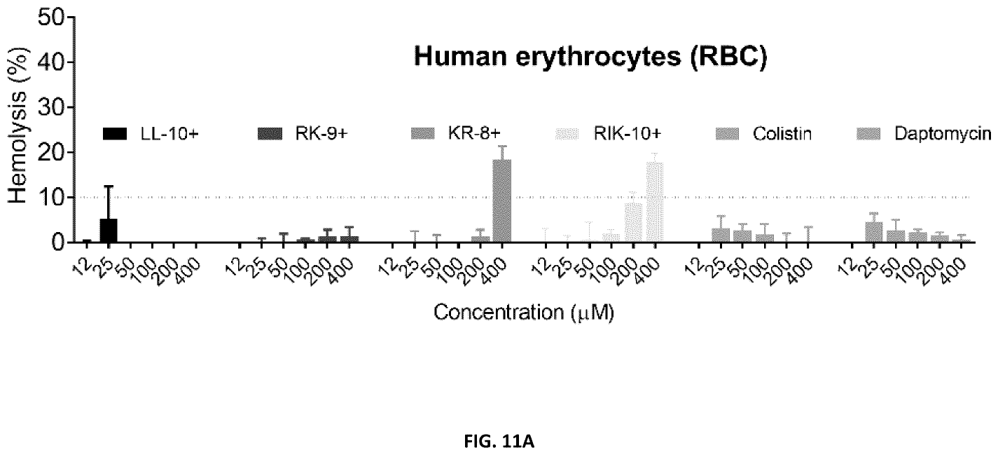

Bacterial growth inhibition kinetics was then followed at varying peptide concentrations. In the case of S. aureus USA300, all the four peptides did not inhibit its growth completely until 16 μM. At 8 μM, KR-8+ (FIG. 10A) and RK-9+(FIG. 10B) were more effective than LL-10+ (FIG. 10C) and RIK-10+ (FIG. 10D). These inhibition curves agree with the MIC values for these peptides against MRSA (Table 8). The growth curves of P. aeruginosa, however, were more distinct for the four peptides. The peptide concentration for complete Pseudomonal inhibition was in the following order: RIK-10+ (4 μM)>KR-8+ (8 μM)>LL-10+ (16 μM)>RK-9+ (32 μM) (FIGS. 10E-10H). These results are consistent with the MIC values in Table 8 and provided additional insight into bacterial growth inhibition kinetics. As RIK-10+ is more effective against Gram-negative bacteria, its bacterial killing kinetics was also compared. At 4×MIC, RIK-10+ was able to eliminate P. aeruginosa in 60 minutes, whereas colistin killed the same bacterium in 30 minutes (FIG. 10I). At the same peptide concentration, it took 90 minutes to kill MRSA USA300 (FIG. 10J). These results indicated stronger antimicrobial power of RIK-10+ to attack Gram-negative pathogen than Gram-positive bacteria, consistent with MIC values (Table 8).

### Effects of pH, Salts, and Serum on Peptide Activity

It is known that media conditions could compromise activity of human LL-37. In human lung, LL-37 becomes less active at pH 6.8 than at pH 7.4. Due to binding, LL-37 can be inactivated by human apolipoprotein A-I (Wang, et al. (1998) J. Biol. Chem., 273(50):33115-8). To further understand antimicrobial potential of these peptides, the effects of pH, physiological salts, and human serum on peptide activity was evaluated. In the case of S. aureus, all the peptides retained activity or even became more active at a basic pH of 8.1, but lost activity at an acidic pH of 6.2 (Table 10). In the presence of 150 mM NaCl, both LL-10+ and RIK-10+ lost activity (MIC>32 μM). In contrast, RK-9+ and KR-8+ retained some activity (MIC 16-32 μM). These peptides retained some activity in the presence of 5% serum, but lost this activity at a higher serum concentration. Under these conditions, however, all the designer peptides appeared to behave better than the parent peptide LL-37 (Table 10). As positive controls, two lipopeptides, C10-KR8 (Lakshmaiah, et al. (2021) ACS Infect. Dis., 77(6):1795-1808) and daptomycin, remained potent against S. aureus USA300 under all these conditions. C10-KR8 is a robust peptide designed by conjugating KR-8 (a short peptide of LL-37) with caprylic acid. As a negative control, colistin was inactive under these conditions against MRSA.

TABLE 10
Effects of pH, salt and serum on antimicrobial activity
of designer peptides against S. aureus USA300.

| | pH | | | NaCl | Human serum | |
|---|---|---|---|---|---|---|
| Peptides | 6.21 | 7.22 | 8.13 | (150 mM) | 5% | 10% |
| RK-9+ | >32 | 8 | 4 | 32 | 32 | >32 |
| LL-10+ | >32 | 16 | 4 | >32 | 32 | >32 |
| RIK-10+ | >32 | 16 | 8 | >32 | 32 | >32 |
| KR-8+ | >32 | 8 | 8 | 16 | 32 | >32 |
| C10-KR8 | 8 | 4 | 2 | 4 | 4-8 | 8 |
| LL-37 | >32 | >32 | 32 | >32 | >32 | >32 |
| Colistin | >8 | >8 | >8 | >8 | >8 | >8 |
| Daptomycin | 0.5-1 | 0.5 | 0.5 | 0.5 | 0.5-1 | 0.5-1 |

To study the effect of bacterial strain, antibacterial activity of these four designer peptides under different pH, salt and serum conditions was evaluated using P. aeruginosa E411-17. As a negative control, daptomycin did not display any activity against P. aeruginosa under all the conditions tested here (Table 11). Interestingly, all the peptides remained pseudomonacidal even at the acidic pH of 6.2 (Table 11). Moreover, the activity of KR-8+ was not compromised in the presence of 150 mM NaCl. As in the case of MRSA, human serum (10%) could reduce the activity of these peptides. Of note, the parent peptide LL-37 retained some activity in 10% serum. Like colistin (positive control), the LL-37 designed C10-KR8 peptide (Lakshmaiah, et al. (2021) ACS Infect. Dis., 77(6):1795-1808) also retained pseudomonacidal activity under these conditions. The version of C10-KR8 was made of D-amino acids, substantially reducing its binding to human serum proteins as proved via mass spectrometry proteomic studies (Lakshmaiah, et al. (2021) ACS Infect. Dis., 77(6):1795-1808).

TABLE 11
Effects of pH, salt and serum on antimicrobial activity
of peptides against P. aeruginosa E411-17.

| | pH | | | NaCl | Human serum | |
|---|---|---|---|---|---|---|
| Peptides | 6.21 | 7.22 | 8.13 | (150 mM) | 5% | 10% |
| RK-9+ | 32 | 8-16 | 4 | >32 | 16-32 | >32 |
| LL-10+ | 16 | 8 | 8 | >32 | 32 | >32 |
| RIK-10+ | 8 | 4-8 | 4 | >32 | 32 | >32 |
| KR-8+ | 8 | 8 | 8 | 8-16 | 32 | >32 |
| C10-KR8 | 16 | 4-8 | 4 | 16 | 8 | 8 |
| LL-37 | >32 | 32 | 16 | 8 | 32 | 32 |
| Colistin | 2 | 1-2 | 1-2 | 2 | 1-2 | 2 |
| Daptomycin | >8 | >8 | >8 | >8 | >8 | >8 |

### Cytotoxicity of the Designed Peptides

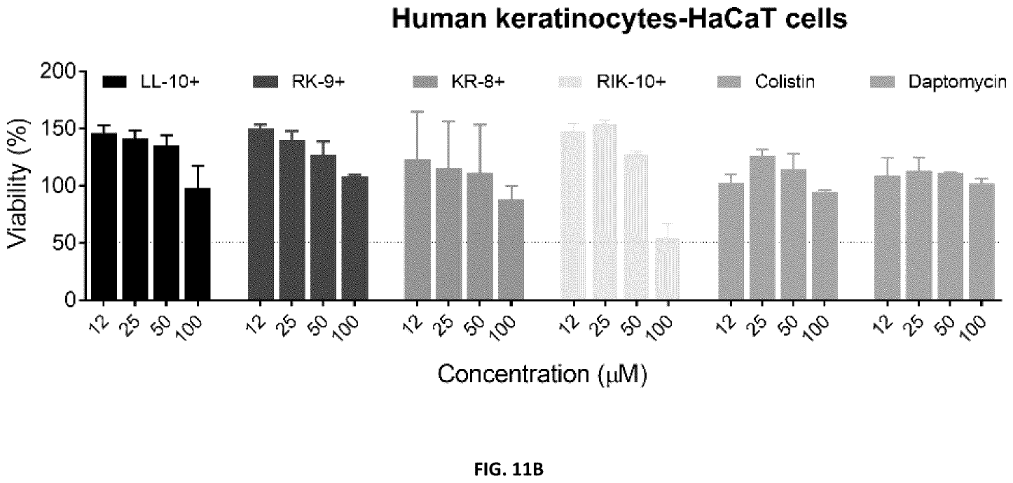

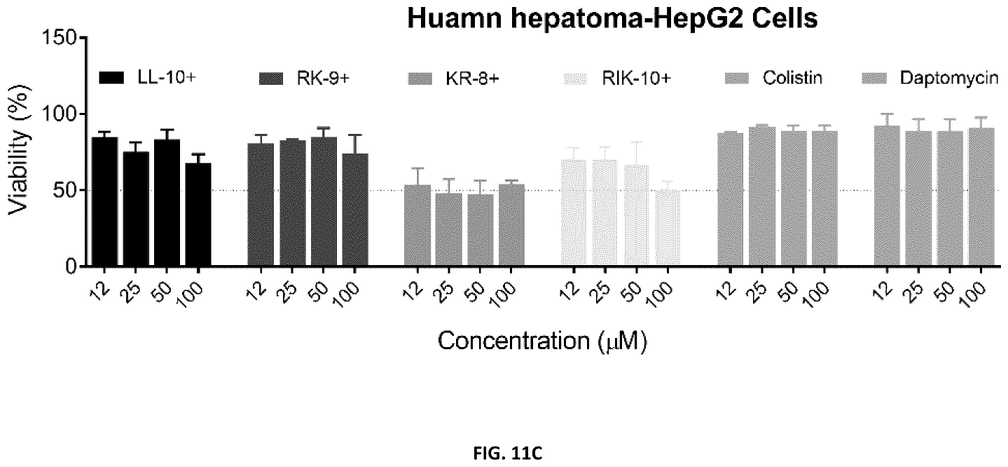

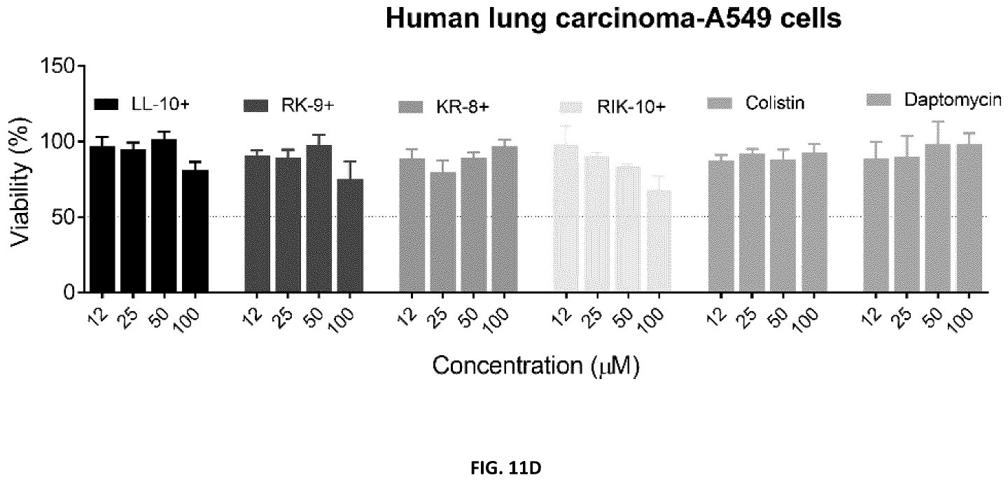

Next, it was determined whether the newly designed peptides are toxic. First, well-established hemolytic assays were used. Like daptomycin and colistin, LL-10+ and RK-9+ did not lyse human red blood cells even at 400 μM. KR-8+ started to show some toxic effect only at 400 uM, whereas RIK-10+ showed a dose-dependent low-level hemolysis only at high peptide concentrations (200 μM). At 400 μM, KR-8+ and RIK-10+ caused 18% hemolysis (FIG. 11A). Since the 50% hemolytic concentrations (HC₅₀) were far greater than 400 μM, all the four designed peptides are highly selective. These short peptides are more selective than its parent peptide LL-37, the major antimicrobial peptide GF-17, and its engineered peptide 17BIPHE2 (Zhang, et al. (2021) Pharmaceuticals 14(12):1245).

Hemolysis of the above peptides was also evaluated after incorporation of Bip (Table 9). Unfortunately, Bip-RIK-10+ became more toxic than RIK-10+ with a HC₅₀ value of ˜30 μM. Although Bip-KR-8+ showed a high HC₅₀ of >200 μM, its HC₁₀ (10% hemolytic concentration) was only 15 μM. The toxicity of these two peptides resulted from the introduction of two Bip amino acids. Bip-LL-10+, with only one Bip, was of some interest with HC₅₀>200 μM and HC₁₀ at 100 μM (Table 9). However, the HC₁₀ values for LL-10+, RK-9+, KR-8+, and RIK-10+ were >400, >400, 300, and 220 μM, respectively, all much less toxic than those Bip-containing peptides.

To provide additional evidence for peptide safety, peptide toxicity was also evaluated using other mammalian cells. In the case of human keratinocytes (HaCaT), LL-10+, RK-9+, and KR-8+ showed little toxicity even at 100 μM similar to daptomycin and colistin. Only RIK-10+ displayed some toxicity at 100 μM with a 50% lethal concentration (LC₅₀) of 100 μM (FIG. 11B). Peptide toxicity to human liver cancer cells (HepG2) commonly used for heptocytoxicity assays was also tested. Like colistin and daptomycin, LL-10+ and RK-9+ showed subtle effects. KR-8+ appeared to be most toxic with LC₅₀ at 12.5 μM. Intriguingly, this effect was not dose dependent as cell viability did not change with increase in peptide concentration. Similar to the effect on human skin cell, RIK-10+ showed a low toxicity below 50 μM with an LC₅₀ at 100 μM. Another two peptides, LL-10+ and RK-9+, were less toxic with LC₅₀>100 μM, comparable to daptomycin and colistin (FIG. 11C). The high cell selectivity of these peptides is also evident in the case of human A549 lung cells with LC₅₀>100 μM (FIG. 11D). These results indicate that three out of the four peptides, LL-10+, RK-9+, and RIK-10+, are safe at the MIC values required to kill these pathogens in Table 8. It appeared that these LL-37 derived peptides might have stimulated the growth of human skin HaCaT but not human liver HepG2 or lung cells at low concentrations.

### Mechanism of Action of the Designed Peptides

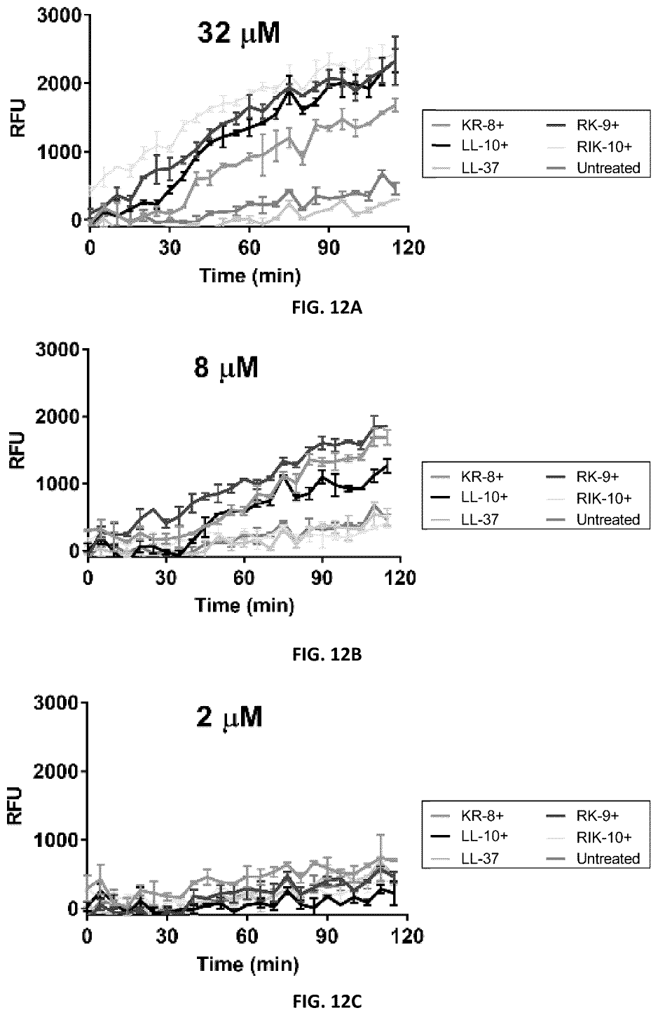

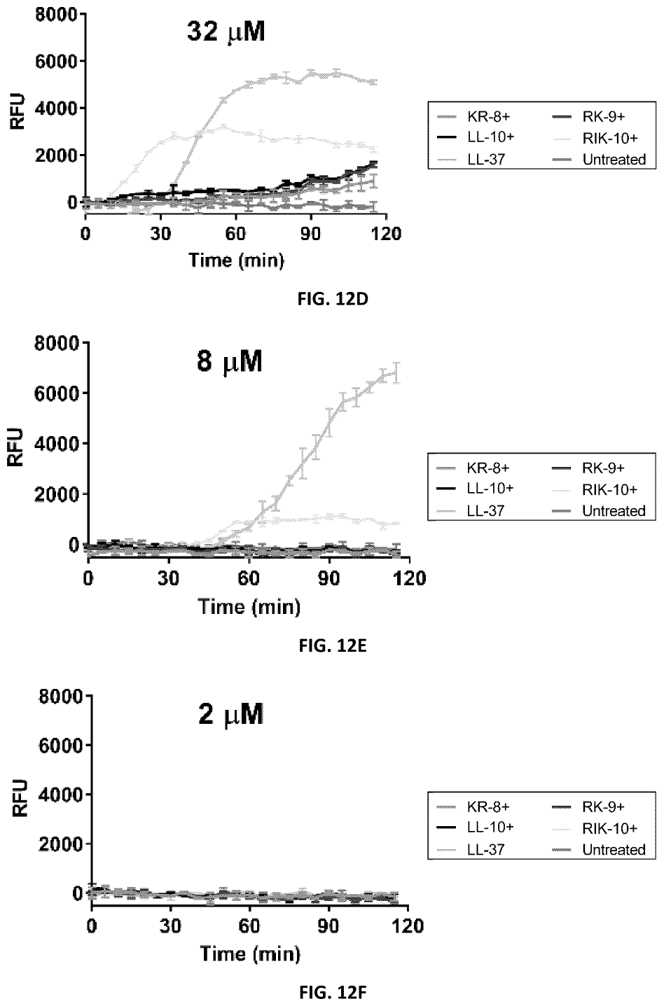

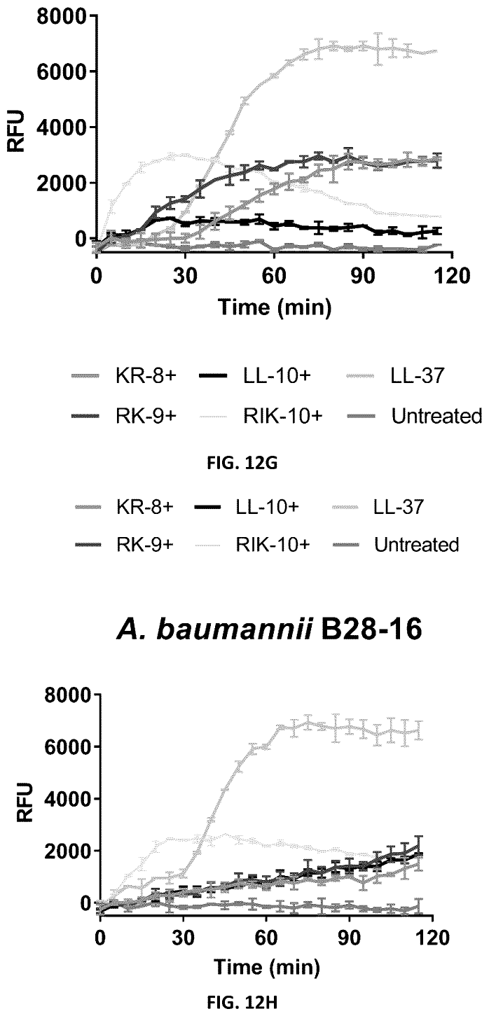

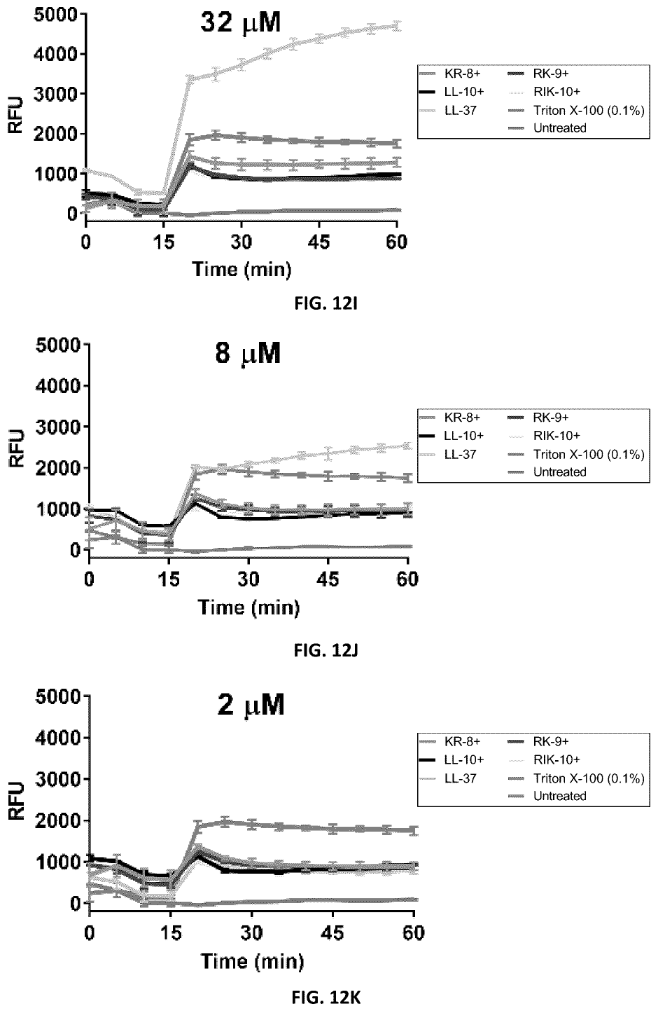

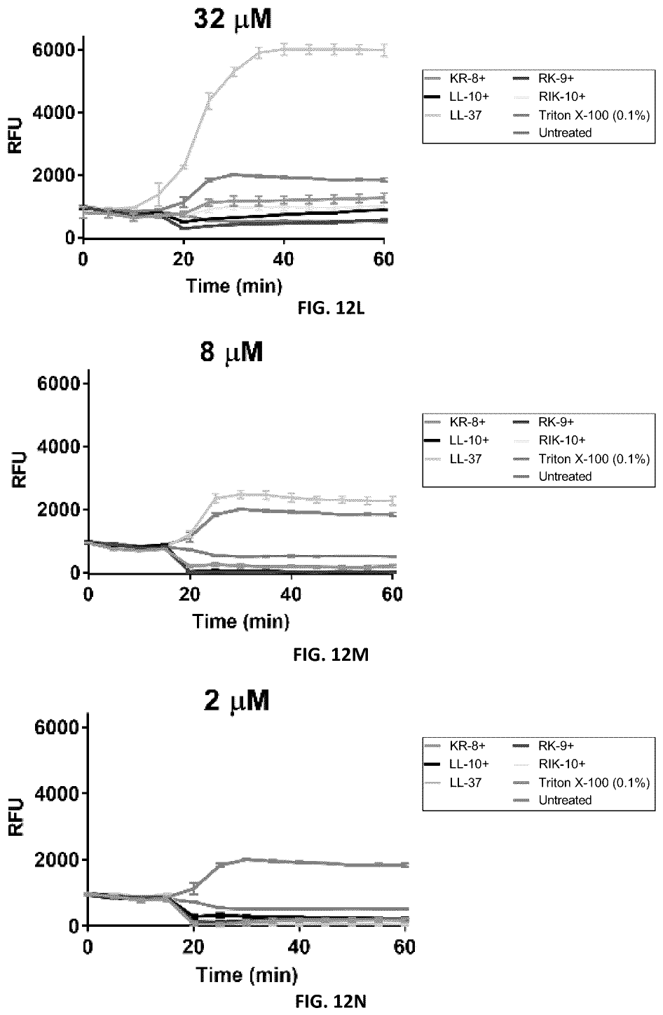

Human LL-37 is known to act on bacterial membranes via the carpet model or by forming the barrel-stave pore or toroidal pore (Oren Z, et al. (1999) Biochem. J., 341(3):501-513; Lee, et al. (2011) Biophys. J. 100 (7):1688; Henzler, et al. (2003) Biochemistry 42(21):6545-6558). The fragments of LL-37, including GF-17 and GI-20, share the same mechanism as indicated by membrane permeabilization (Zhang, et al. (2021) Pharmaceuticals 14(12):1245). Since LL-10+, RK-9+, KR8+, and RIK-10+ are all designed based on LL-37, it is likely that these short peptides also target bacterial membranes. To confirm this, membrane permeabilization experiments were conducted with and without the peptide. In this experiment without the peptide, the indicator dye could not enter bacteria, associate with DNA, and emit fluorescence (FIGS. 12A-12F). In the presence of the designed peptides, an increase in fluorescence implied membrane permeabilization of S. aureus USA300. In contrast, LL-37 failed to do so since it is known to be inactive in TSB (FIG. 12A). Together with FIGS. 12B and 12C, a peptide dose-dependent membrane permeabilization (1-32 μM) was evident. With an increase in peptide concentration till 8 μM, fluorescence increase started to be evident with RK-9+ at the top. This remained the same until 32 μM where RIK-10+ became slightly more powerful than RK-9+. To study the effect of bacterium, P. aeruginosa was used. At 8 μM, RIK-10+ started to permeate bacterial membranes, while the effects of other peptides were marginal. Despite some delay, LL-37 reached a much higher level of fluorescence than RIK-10+. The same trend recurred at 32 μM. It appeared that peptide membrane permeation was related to MIC values. To verify this, E. coli and A. baumannii were also studied. Remarkably, similar membrane permeabilization of E. coli and A. baumannii by LL-37 was observed, which shows similar activity against these Gram-negative bacteria (FIGS. 12G-12H). Consistently, RIK-10+ was strongest, whereas LL-10+ was the weakest in the case of E. coli. In all the cases, RIK-10+ was able to permeate bacterial membranes faster than LL-37 although it did not reach the magnitude achieved by its parent peptide. RIK-10+ is superior to other designed peptides in permeating the membranes of all the three Gram-negative bacteria. This is interesting considering only RIK-10 was active against E. coli before enhancement. These results underscore the significance of the RIK-10 sequence derived from the core antimicrobial region of LL-37 in stopping the infection of Gram-negative pathogens (FIG. 8).

### Cationic AMPs May Also Depolarize Bacterial Membranes

This effect was measured in PBS buffer primed with glucose. Under such a condition, LL-37 was observed to be most powerful in membrane depolarization, even more effective than the positive control Triton®-X100 at 8 μM and above for both S. aureus (FIGS. 12I-12K) and P. aeruginosa (FIG. 12L-12N). Compared to LL-37 or Triton®-X100, the membrane depolarization effects of the designed ultrashort peptides were generally weak. At 32 μM, KR-8+ was slightly better than other short peptides in the case of S. aureus. All the designed peptides only weakly depolarized bacterial membranes of P. aeruginosa even at 32 μM. Again KR-8+ was at the top of the four designed peptides in this experiment. Taken together, without being bound by theory, membrane permeabilization appeared to be the main mechanism of action for these designed short peptides. In contrast, both membrane permeation and depolarization play an evident role in the bacterial killing of their parent peptide LL-37.

### Antibiofilm Capability of the Four Designed Peptides

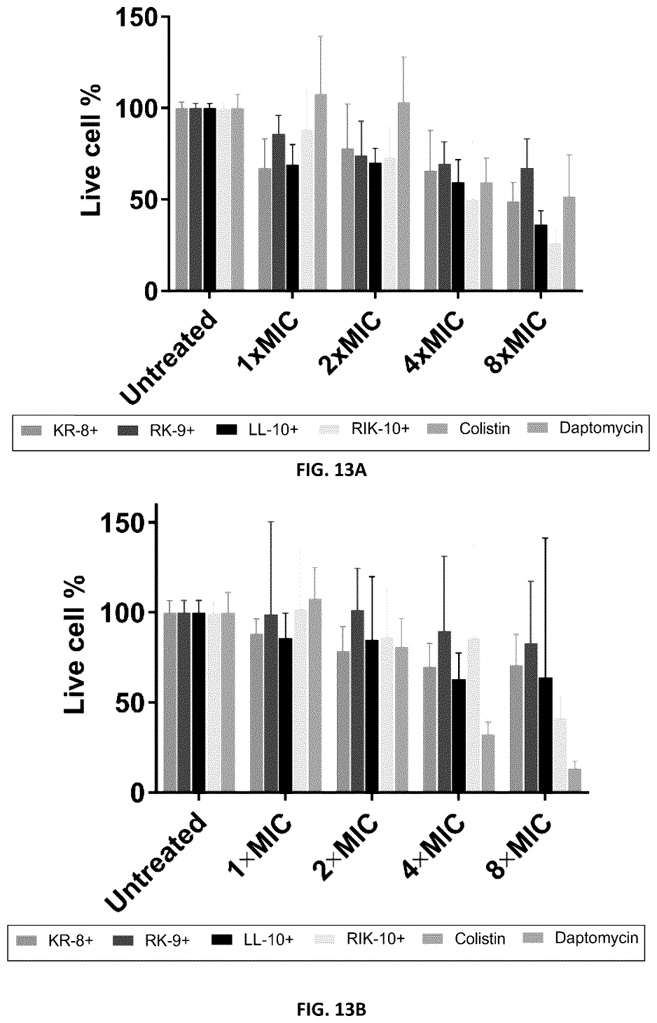

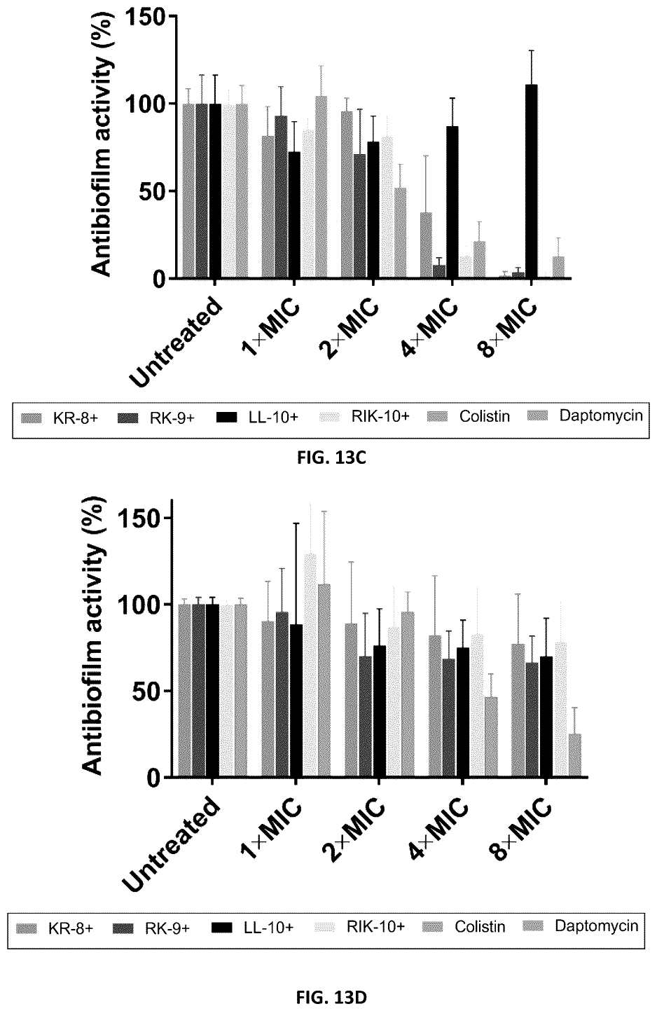

As most of bacteria are in the biofilm state, antibiofilm activity of the four peptides designed based on ultrashort LL-37 segments was compared. Both S. aureus USA300 and P. aeruginosa E411-17 were included in bacterial attachment (FIGS. 13A-13B) and preformed biofilm disruption experiments (FIGS. 13C-13D). In the case of MRSA, RIK-10+at 8 fold of its MIC was found to be more potent than the other three peptides against bacterial attachment, the first step in biofilm formation. Importantly, RIK-10+ was also more potent than daptomycin treated at 8×MIC (FIG. 13A). A dose-dependent antibiofilm effect was also observed for 24-hour preformed biofilms of MRSA. At 4×MIC, peptides LL-10+ and KR-8+ were less effective than RK-9+ and RIK-10+. The latter two appeared to be more potent than daptomycin at 4×MIC and 8×MIC (FIG. 13C).

In the case of P. aeruginosa, these peptides were comparable in inhibiting bacterial attachment at 2×MIC or below (FIG. 13B). At 8×MIC, RIK-10+ appeared to be slightly more effective than other peptides. However, they were all less effective than colistin at 4× or 8×MIC. The biofilm-disruption ability of these peptides was also tested. The dose-dependent effect on P. aeruginosa (FIG. 13D) was less pronounced than that on MRSA (FIG. 13C). As demonstrated for other LL-37 peptides (Mishra, et al. (2016) ACS Med. Chem. Lett., 77 (1):117-121), these designer small peptides can inhibit biofilm formation probably via direct bacterial killing at and above MIC and coating anionic bacterial surface below MIC to block initial attachment.

### In Vivo Antibiofilm Activity of the Four Peptides in a Murine Wound Model

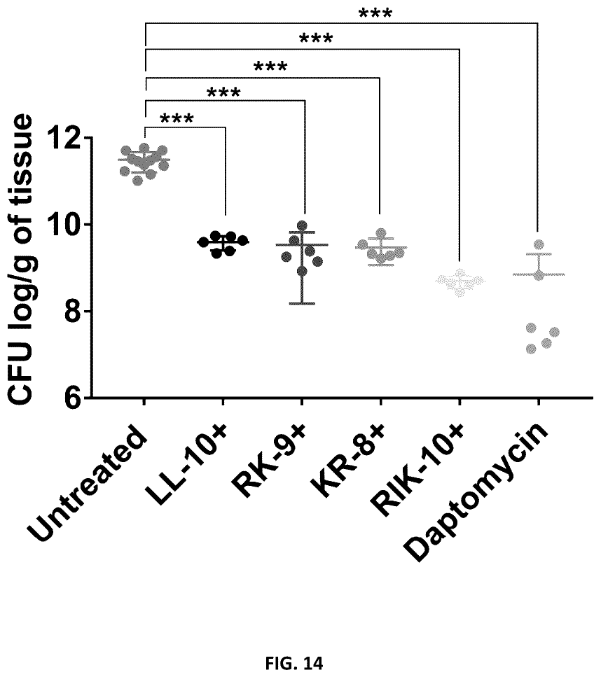

One major hurdle for wound healing is bacterial infection and biofilm formation. The current APD has annotated 30 AMPs to have wound healing effect (Wang, G. (2023) Protein Science 32(10):e4778). Human LL-37 is a typical example that can eliminate bacteria and promote wound healing (Duplantier, et al. (2013) Front. Immunol., 4:143; Heilborn, et al. (2003) J. Invest. Dermatol., 120(3):379-89; John, et al. (2023) Adv. Funct. Mater., 33(1):2206936). To further compare the antibiofilm ability of the four designer peptides, their in vivo efficacy in treating the 24-hour preformed biofilms of S. aureus USA300 in murine wounds was also evaluated. These peptides, when directly loaded to the wound, were able to reduce the bacterial burden by 2-3 logs after one treatment for 24 hours (FIG. 14). They showed similar effects, consistent with similar MIC values against MRSA (Table 8). The antibiofilm effects of these peptides were also comparable to daptomycin in the same animal model. After formulation, an eight-residue LL-37 mimicking peptide can further eliminate bacterial pathogens and shows a synergistic effect with nanomaterial in promoting wound healing (John, et al. (2023) Adv. Funct. Mater., 33(1):2206936). The small size of these designer peptides substantially reduces the production cost compared to LL-37.

### Membrane-Bound Structure of RIK-10+ Determined by 2D NMR Spectroscopy

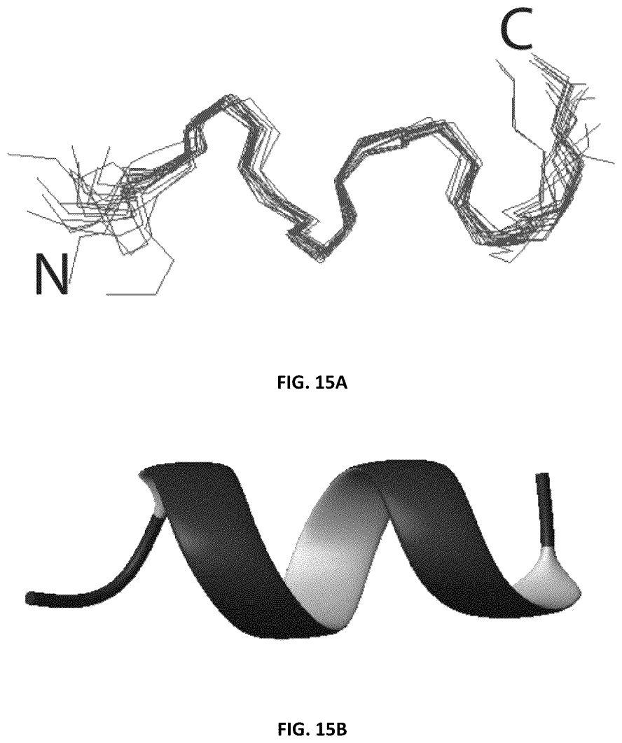

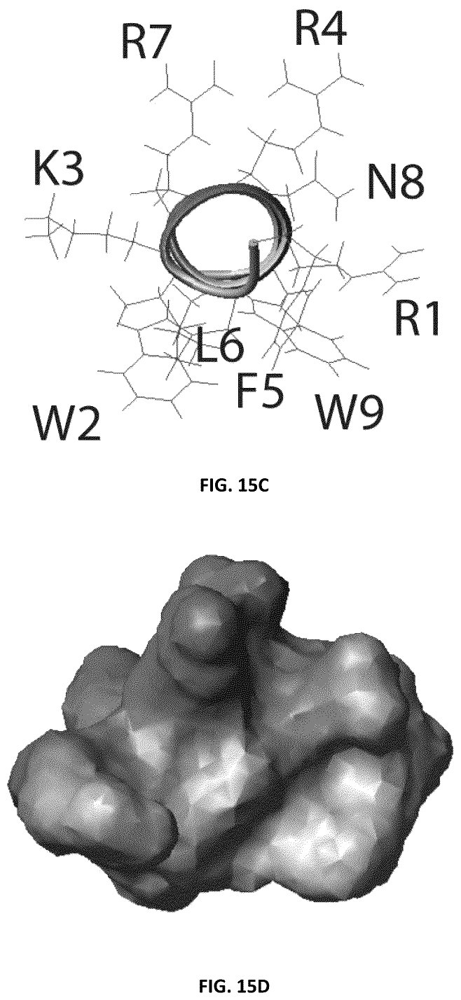

LL-37 has a long helix covering residues 1-31 (FIG. 8A) (Wang, G. (2008) J. Biol. Chem., 283(47):32637-32643). The helical structure is retained for its fragments in the membrane bound state or even in PBS (Wang, G. (2008) J. Biol. Chem., 283(47):32637-32643; Li, et al. (2006) J. Am. Chem. Soc., 128(17):5776-5785; Zhang, et al. (2021) Pharmaceuticals 14(12):1245). It can be projected that the small peptides designed here are potentially helical as well (FIG. 9). To substantiate this, the 3D structure of RIK-10+ was determined, which is a more potent anti-Gram-negative peptide (Table 8 and FIGS. 10A-10H). Since the peptide acted on bacterial membrane (FIGS. 12A-12F), lipid micelles of dodecylphosphocholine (DPC) were utilized to mimic bacterial membranes (Bosch et al. (1980) BBA—Biomembranes, 603(2):298-312). This deuterated lipid could give a signal dispersion comparable to that of dioctanoylphosphatidylglycerol where deuterated version is not yet available (Keifer, et al. (2004) Anal. Biochem., 331(1):33-39). At a peptide:lipid ratio of 1:60, one peptide was bound to one micelle. This sample enabled us to determine the membrane-bound structure of RIK-10+ by using the established solution NMR method (Wuthrich, K. (1986) Europhysics News 17(1):11-13). Peptide proton signals were assigned using the Wuthrich sequential assignment method. 101 NOE restraints were obtained for this 10-residue peptide. In addition, 16 dihedral angle restraints were generated for residues 3-10 based on chemical shifts, which are known to improve structural quality (Cornilescu, et al. (1999) J. Biomol. NMR 13(3): 289-302; Wang, et al. (2005) J. Biol. Chem., 280(7):5803-5811). The NMIR-determined 3D structure is presented in FIG. 15A. The structure was determined to high quality with 90% of the backbone angles in the most favored Ramachandran region and 10% in the additional allowed region. Residues 3-10 were found to be helical with a superimposed backbone RMSD of 0.32 Å (FIGS. 15 A and 15B). When viewed from the end of the structure, the amphipathic nature of RIK-10+ was evident with W2, F5, L6, and W9 forming the hydrophobic surface (FIG. 15C). The amphipathic pattern of the structure could also be seen in the potential surface with basic charge in blue and hydrophobic amino acids in light purple (FIG. 15D). A further analysis of the 2D NOESY spectra revealed that the peptide sidechains directly interacted with residual protons from DPC. There were intermolecular NOE cross peaks from well-resolved DPC protons (0.6 ppm) to sidechains of R1, W2, K3, R4, and W9, confirming interfacial location of these amino acids in the peptide-lipid complex.

### Charge and Hydrophobic Density Plots of the Designed Peptides

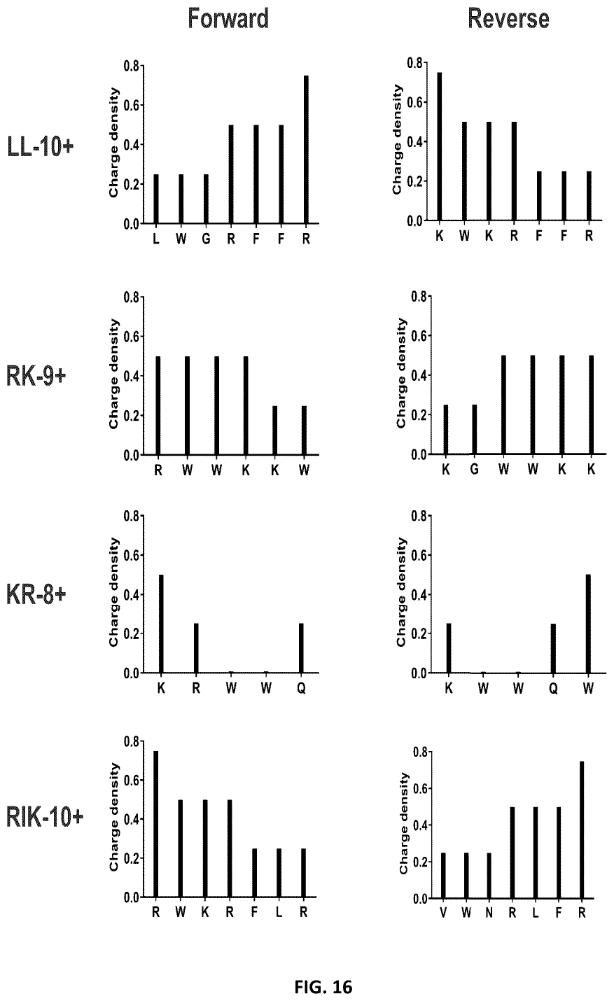

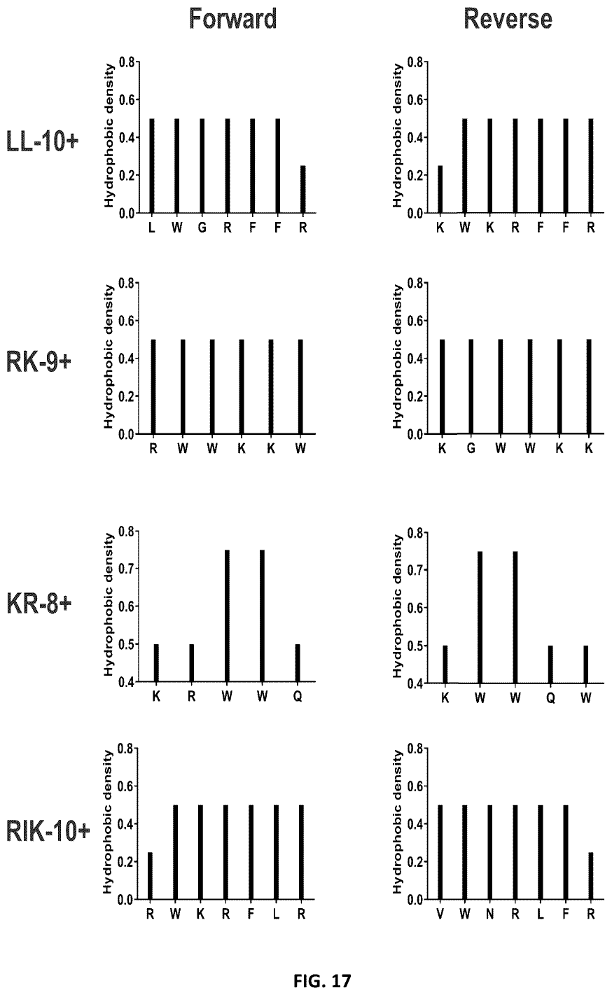

Because basic charge and hydrophobicity are the two most important physiochemical parameters of amphipathic helical peptides, both charge and hydrophobic densities for the four peptides were calculated. As α-helix has 3.6 residues per turn, it was rounded up to a window size of 4. For charge density calculations, the number of arginine (R) and lysine (K) was summed and divided by the window size (there is no histidine in these sequences). Likewise, the hydrophobic density per residue was calculated by summing all the eight hydrophobic amino acids (A, V, L, I, F, M, C, and W) defined in the APD (Wang, et al. (2016) Nucleic Acids Res., 44(D1):1087-1093; Wang, G. (2023) Protein Science 32(10):e4778) and divided by the same window size of 4. A computing R program was written to facilitate these calculations. Both charge and hydrophobic density calculations started from the N-terminal amino acid of each sequence and ended when it reached the last four residues at the C-terminus. Charge plots (FIG. 16) were focused on since the hydrophobic plots (FIG. 17) were rather homogeneous except for KR-8+. For LL-10+, charge density increased from the N-terminus to the C-terminus with the highest at 0.75. In contrast, the plots for another three peptides were higher at the N-termini. Interestingly, RK-9+, KR-8+, and RIK-10+ (MIC 4-8 μM) were all more potent against E. coli than LL-10+ (MIC 32 μM). It appeared that the location of the high-density charge at the N-terminus was important in E. coli killing. To confirm this, reverse peptide sequences were tested. LL-10+ and RIK-10+ were selected as they presented an exactly opposite charge plots (FIG. 16). These two peptides were chemically synthesized and subjected to antibacterial testing. Remarkably, LL-10+ became much more potent (MIC 4 μM) against E. coli after sequence reversal (named retroLL-10+). In contrast, RIK-10+ after sequence reversal became much weaker (MIC 32 μM in Table 12). As anticipated, their charge density plots were also reversed (FIG. 16). Such a difference is not clear in the helical wheel plots after sequence reversal (FIG. 9). These results confirmed the importance of the N-terminal location of the highly charged regions for antibacterial activity against Gram-negative bacteria.

TABLE 12
Effects of sequence reversal on antibacterial activity of designed
peptides. All the peptides are C-terminally amidated. K/R are bolded
to show the different charge distribution patterns in these peptide
sequences. Peptide activity is represented as minimal inhibitory
concentration (MIC) in μM.

| | | | S. | E. | | |
|---|---|---|---|---|---|---|
| | | SEQ | aureus | coli | P. | K. |
| | Peptide | ID | USA | E423 | aeruginosa | pneumoniae |
| Name | sequence | NO | 300 | -17 | E416-17 | E406-17 |
| LL-10+ | LWGRFFRKWK | 30 | 16 | 32 | 8 | >32 |
| RetroLL- | KWKRFFRGWL | 34 | 8 | 4 | 4 | >32 |
| 10+ | | | | | | |
| RIK-10+ | RWKRFLRNWV | 33 | 16-32 | 4-8 | 4 | 32 |
| RetroRIK- | VWNRLFRKWR | 35 | 32 | 32 | 16 | >32 |
| 10++ | | | | | | |

### Charge and Hydrophobic Plots Shine New Light on Nature's Design of Human Cathelicidin

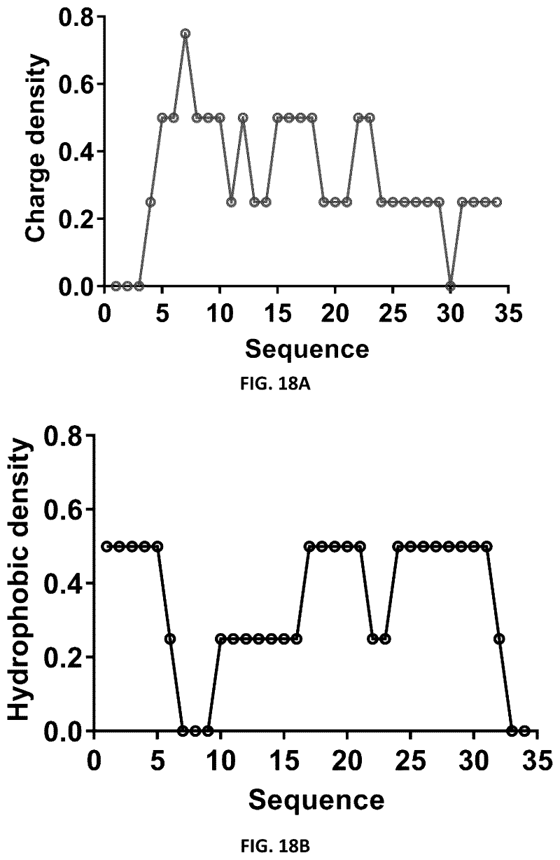

Since RIK-10 is the shortest antibacterial unit of LL-37, the charge and hydrophobic plots of LL-37 were examined. In the charge-density plot (FIG. 18A), the magnitude of charge gradiently decreased from the N-terminal to the C-terminal region, providing one interpretation for its antibacterial activity against Gram-negative bacteria. The hydrophobic density plot of LL-37 (FIG. 18B) is also interesting. There were three 50% hydrophobic zones: residues 1-4, 17-21, and 24-31. A clear hydrophobic valley existed between the first and second zones due to the presence of hydrophilic S9 on the hydrophobic surface as observed in the 3D structure of LL-37 (Wang, G. (2008) J. Biol. Chem., 283(47):32637-32643). Another two 50% hydrophobic zones with a dip at residues 22-23, when combined, correspond exactly to the major antimicrobial region discovered from NMR structural studies (Li, et al. (2006) J. Am. Chem. Soc., 128(17):5776-5785). Overall, the charge and hydrophobic plots of LL-37 are rather complementary to each other since a highly charged region is achieved at the expense of hydrophobic amino acids (FIGS. 18A-18B).

Charge Density Plots for Antimicrobial Peptides Against Gram-Negative Bacteria Obtained from the APD

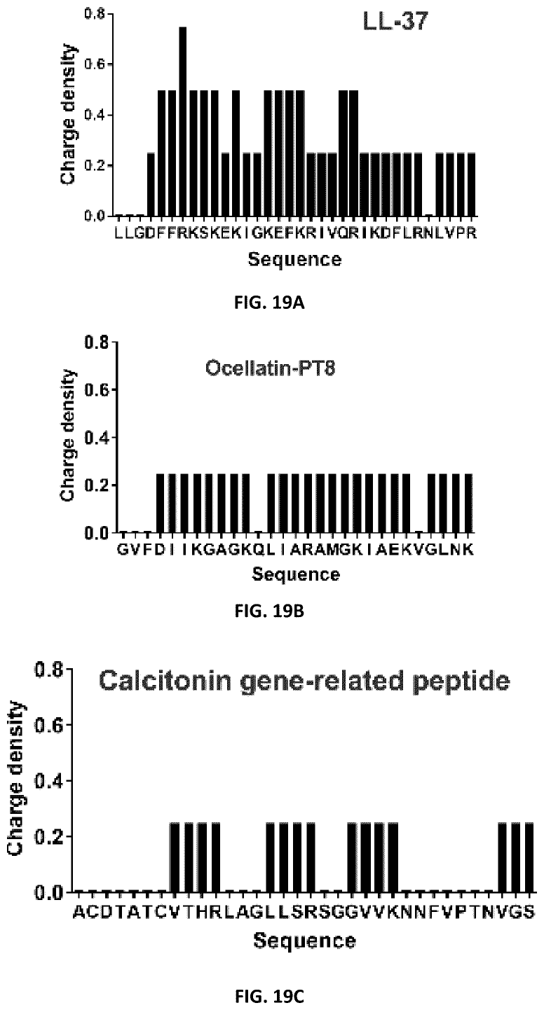

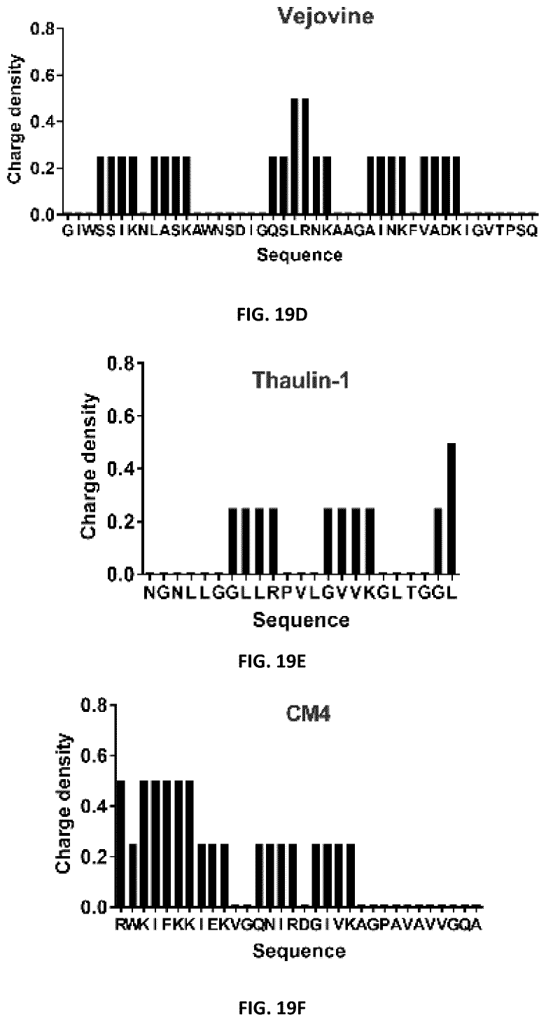

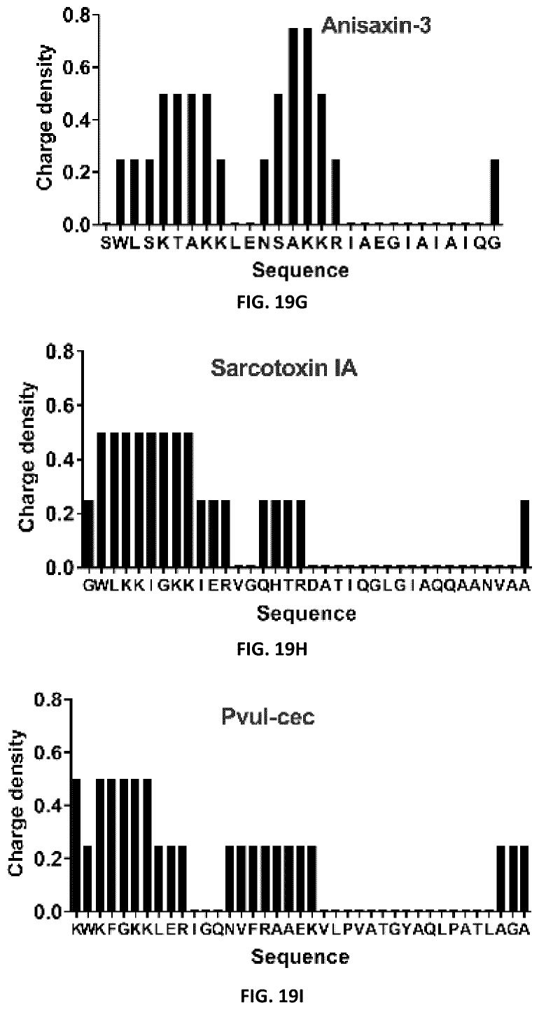

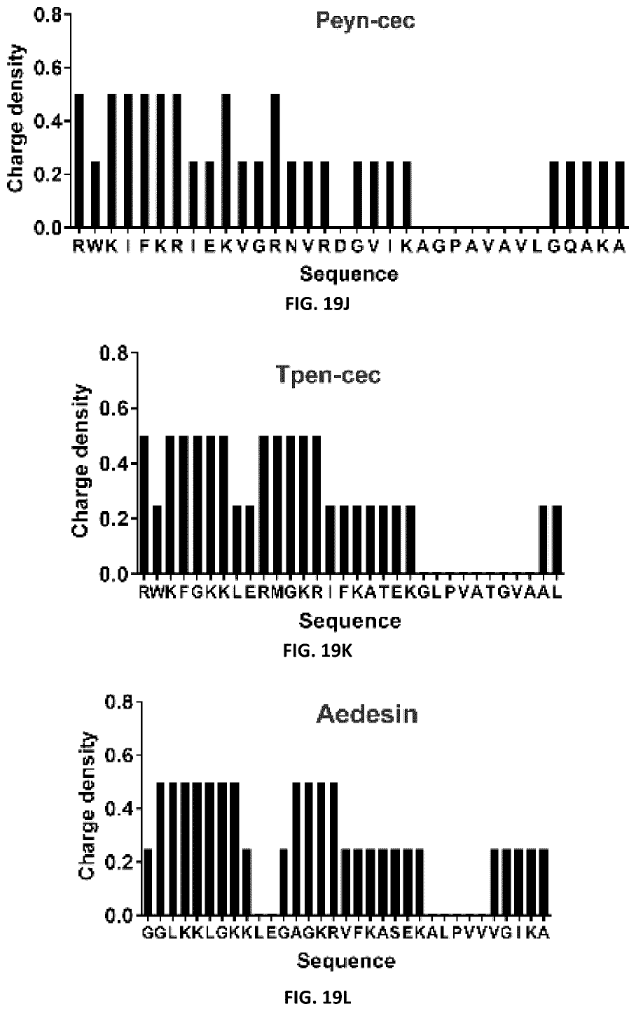

To identify additional examples, the APD was searched for peptides with activity against Gram-negative only. The following search criteria were applied: (1) active against Gram-negative bacteria only; (2) peptide length less than 50 amino acids; (3) peptides containing both arginine and lysine; (4) peptides with known helical structure; and (5) natural AMPs. These database filters led to a total of 11 peptides in Table 13. Of note, numerous peptides deployed more basic amino acids at the N-termini. This can be viewed more clearly from the charge density plots for seven peptides (FIG. 19F-19L). For comparison, LL-37 is provided in FIG. 19A. Biologically, these peptides are active against Gram-negative pathogens such as E. coli (Table 13). While the charge density plot of amphibian ocellatin-PT8 (FIG. 19B) showed a relatively even distribution of positively charged R and K along the sequences, the basic amino acids of amphibian thaulin-1 (FIG. 19E) are mostly located at the C-terminus. Such different charge distribution patterns may explain in part why they showed weak activity. However, two peptides with 4-5 charge clusters (FIG. 19C-19D) showed good activity against E. coli. Hence, other charge distribution patterns such as an even distribution are also useful, enriching charge distribution patterns in natural AMPs.

TABLE 13

| | | | SEQ | | |
|---|---|---|---|---|---|
| APD | | | ID | MIC against | |
| ID | Peptide | Amino acid sequence | NO | E. coli | Ref |
| 2600 | Ocellatin- | GVFDIIKGAGKQLIARAM | 36 | 60 μM | [1] |
| | PT8 | GKIAEKVGLNKDGN | | | |
| 1476 | Calcitonin | ACDTATCVTHRLAGLLS | 37 | 2.1 μg/ml | [2] |
| | gene- | RSGGVVKNNFVPTNVGS | | | |
| | related | KAF | | | |
| | peptide | | | | |
| 1753 | Vejovine | GIWSSIKNLASKAWNSDI | 38 | 4.4-20 μM | [3] |
| | | GQSLRNKAAGAINKFVA | | | |
| | | DKIGVTPSQAAS | | | |
| 2765 | Thaulin- | NGNLLGGLLRPVLGVVK | 39 | 62.5 μg/ml | [4] |
| | 1 | GLTGGLGKK | | | |
| 1259 | CM4 | RWKIFKKIEKVGQNIRDG | 40 | 12 μM | [5] |
| | | IVKAGPAVAVVGQAATI | | | |
| 3409 | Anisaxin- | SWLSKTAKKLENSAKKR | 41 | 0.5-1.0 μM | [6] |
| | 3 | IAEGIAIAIQGGPR | | | |
| 230 | Sarcotoxin | GWLKKIGKKIERVGQHT | 42 | 0.2-6.3 | [7] |
| | IA | RDATIQGLGIAQQAANV | | μg/ml | |
| | | AATAR | | | |
| 3746 | Pvul- | KWKFGKKLERIGQNVFR | 43 | 8 μg/ml | [8] |
| | cec | AAEKVLPVATGYAQLPA | | | |
| | | TLAGAKQG | | | |
| 3745 | Peyn- | RWKIFKRIEKVGRNVRD | 44 | 8 μg/ml | [8] |
| | cec | GVIKAGPAVAVLGQAKA | | | |
| | | LGK | | | |
| 3747 | Tpen- | RWKFGKKLERMGKRIFK | 45 | 8 μg/ml | [8] |
| | cec | ATEKGLPVATGVAALAR | | | |
| | | G | | | |
| 3507 | Aedesin | GGLKKLGKKLEGAGKR | 46 | 2-4 μg/ml | [9] |
| | | VFKASEKALPVVVGIKAI | | | |
| | | GK | | | |

Helical antimicrobial peptides (<50 aa, K and R-containing) against Gram-negative bacteria. Data obtained from the antimicrobial peptide database (aps.unmc.edu) by setting the following filters: length <50 aa, K and R containing, helical structure, active against Gram-negative bacteria only. The peptides were sorted based on net charge, which increases from top to bottom of the table. Notably, highly charged regions are mostly located at the N-termini of these peptides (APD ID bolded). References: [1] Marani, et al. (2015) J. Nat. Prod., 78(7): 1495-1504; [2] El Karim, et al. (2008) J. Neuroimmunol., 200: 11-16; [3] Hernandez-Aponte, et al. (2011) Toxicon., 57(1): 84-92; [4] Marani, et al. (2017) Gene 605: 70-80; [5] Tu, et al. (1989) Sci. China B 32: 473-480; [6] Roncevic, et al. (2022) Acta Biomater., 146: 131-144; [7] Okada, et al. (1985) J. Biol. Chem., 260(12): 7174-7177; [8] Guo, et al. (2023) Insects 14(10): 794; [9] Godreuil, et al. (2014) PLOS One 9(8): e105441.

Antimicrobial peptides are host defense molecules of innate immunity that rapidly stop invading microbes. Despite structural diversity, they have been unified into four classes: linear (e.g., human LL-37), sidechain-linked (e.g., defensins), sidechain-backbone-linked (e.g., daptomycin), and backbone-linked (i.e., cyclotides) peptides based on chain connection patterns (Wang, G. (2022) Methods in Enzymology 663:1-18). Among them, the linear peptides are extensively studied and some important discoveries have been made. For over 1000 amphibian peptides, which are mostly helical after binding to bacterial membranes, net charge (mainly due to lysine) increases whereas hydrophobic ratio (mainly due to leucine) decreases with the increase in peptide length (Wang, G. (2020) Antibiotics 9(8):491). In addition, peptides with low cationicity and high hydrophobicity tend to kill primarily Gram-positive bacteria (Mishra, et al. (2019) Proc. Natl. Acad. Sci., 116(27):13517-13522; Narayana, et al. (2020) Proc. Natl. Acad. Sci., 117(32):19446-9454). However, a high net charge is frequently observed for peptides against Gram-negative bacteria (Wang, G. (2020) Protein Sci., 29:8-18; Wang, G. (2020) Antibiotics 9(8):491). Consistent with this database finding, GF-17, a wide-spectrum human LL-37 peptide (Wang, et al. (2012) Antimicrob. Agents Chemother., 56(2):845-856), was converted into narrow-spectrum peptides that are active against either only Gram-positive or Gram-negative bacteria (Wang, G. (2008) J. Biol. Chem., 283(47):32637-32643; Wang, et al. (2018) Adv. Biosyst., 2(5):1700259). This study has advanced the knowledge by identifying sequence features that determine peptide activity against Gram-negative bacteria. This feature was identified by designing multiple short and active peptides because longer peptides such as human cathelicidin LL-37 can contain different functional regions, which could complicate the analysis. After activity enhancement of the four small segments of LL-37 (FIG. 8), all the peptides achieved a net charge and hydrophobic content comparable to the majority of natural AMPs in the APD. Interestingly, these short peptides demonstrated similar activity against MRSA both in vitro (Table 1) and in vivo (FIG. 14). Nevertheless, they vary in activity against Gram-negative bacteria. A gradient increase of peptide activity against Gram-negative bacteria from LL-10+, RK-9+, KR-8+, to RIK-10+ was noticed. It appears that the antibacterial activity difference is related to Boman index with higher values for KR-8+ and RIK-10+ than LL-10+ and RK-9+ (Table 7). Structurally, these peptides are anticipated to be helical (FIG. 9) like the parent peptide LL-37 (FIG. 8A) as the changes made are largely conservative. This is indeed the case after the 3D structure of RIK-10+ by 2D NMR spectroscopy was completed.

Because basic and hydrophobic amino acids are the two most important parameters for cationic AMPs (Johansson, et al. (1998) J. Biol. Chem., 273(6):3718-24; Wang, G. (2008) J. Biol. Chem., 283(47):32637-32643; Xhindoli, et al. (2016) BBA 1858(3):546-566; Wang, et al. (2019) Adv. Exp. Med. Biol., 1117:215-240; De Breij, et al. (2018) Sci. Transl. Med., 10(423):eaan4044), charge and hydrophobic density plots were calculated to gain additional insight into peptide activity differences against Gram-negative bacteria. While the hydrophobic plots are similar for the four designed short peptides, charge density plots differ. Remarkably, three of the designed peptides deployed cationic amino acids primarily at the N-terminus, corresponding to stronger activity against E. coli (Table 8). In the case of LL-10+, a peptide derived from the N-terminus of LL-37 (FIG. 8), its highly charged region was located at the C-terminus, corresponding to the poorest activity in inhibiting E. coli. The location of a highly basic region at the N-terminus of these short peptides appears important for antimicrobial activity against Gram-negative pathogens. Subsequently, this finding was validated by making sequence reversed peptides. As anticipated, the sequence reversal increased the activity of LL-10+, but decreased the activity of RIK-10+ against E. coli (Table 12). The finding here is in line with the two distinct amphipathic peptides: horine and verine (Narayana, et al. (2020) Proc. Natl. Acad. Sci., 117(32):19446-9454). Horine, with the hydrophobic motif WWW at the N-terminus, kills mainly Gram-positive MRSA. In contrast, verine, with triple basic amino acids RRR at the N-terminus, kills both Gram-positive and Gram-negative bacteria. The deployment of high cationcity in the case of LL-37 was also observed. These results underscore the importance of a proper positioning of basic amino acids in peptide sequences for potency against Gram-negative pathogens. Indeed, multiple natural sequences from invertebrates (e.g., insects) in the antimicrobial peptide database share a similar charge distribution pattern (FIG. 19). Most of 34 insect cecropins place multiple lysine, arginine, histidine, or both lysine and arginine at the N-terminus. Cecropins could preferentially eliminate Gram-negative bacteria over Gram-positive bacteria (Boman, H. G. (2003) J. Intern. Med., 254:197-215; Mathew, et al. (2021) ACS Infect. Dis., 7(8):2536-2545; Guo, et al. (2023) Insects 14(10):794). Likewise, 12 insect moricins, with broad bactericidal activity, take advantage of a similar design for host defense (Hara, et al. (1995) J. Biol. Chem., 270(50):29923-7). Insertion of 3 lysines to the N-terminal region of ctriporin drastically increased peptide activity against E. coli, P. aeruginosa, and K. pneumoniae by 8-16 fold (Luo, et al. (2024) Toxins 16(3):156). Hence, the placement of high cationicity at the peptide N-termini constitutes a natural strategy to control invading Gram-negative pathogens. Driven by electrostatic interactions, such a molecular design would enable the host defense bullet to more effectively reach the targeted pathogen like an arrow.

Short active peptides within LL-37 have been sought via structural studies to reduce the production cost (Wang, G. (2008) J. Biol. Chem., 283(47):32637-32643; Li, et al. (2006) J. Am. Chem. Soc., 128(17):5776-5785). A series of antimicrobial fragments from LL-37 was identified with KR-12 being the smallest (FIG. 1B) (Wang, G. (2008) J. Biol. Chem., 283(47):32637-32643). KR-8 only became active after MHB medium dilution. The peptide designed based on KR-8 was named LL-37mini. Among the four ultrashort natural segments of human LL-37 (FIG. 8), however, only RIK-10 is able to inhibit the growth of E. coli without media dilution. After peptide activity enhancement, it is remarkable that RIK-10+ remains more potent than other small designer peptides. Since both KR-8 and RIK-10 with an overlapping sequence are derived from the same major antimicrobial region of LL-37, KR-8+ is referred to as LL-37mini1 and RIK-10+ is referred to as LL-37mini2, which has gained numerous merits: being short, potent, and highly selective. These LL-37mini peptides are 2-4 residues shorter than KR-12, the initial minimal antibacterial peptide (Wang, G. (2008) J. Biol. Chem., 283(47):32637-32643). Taken together, this study has not only deepened the fundamental understanding of natural AMPs but also brought about effective peptides for developing novel antimicrobials to combat difficult-to-kill Gram-negative pathogens.

LL-37 based peptide design utilized the active regions discovered from either structural biology or peptide library (Braff, et al. (2005) J. Immun., 174(7):4271-4278; Molhoek, et al. (2009) Biol. Chem., 390(4):295-303; Nagant, et al. (2012) Antimicrob. Agents Chemother., 56(11):5698-5708; Li, et al. (2006) J. Am. Chem. Soc., 128(17):5776-5785; De Breij, et al. (2018) Sci. Transl. Med., 10(423): eaan4044). This study took another step in peptide design by dissecting the helical region of human cathelicidin into overlapping ultrashort fragments with 10 amino acids or less. The statistical differences between natural and synthetic AMPs (Wang, G. (2023) Protein Sci., 32(10):e4778) guided the design. A similar net charge, hydrophobic ratio, and length for these designer peptides (LL-10+, RK-9+, KR-8+, and RIK-10+) might be responsible for their comparable anti-MRSA activity. However, they showed different MICs against Gram-negative bacteria with RIK-10+ being strongest. This is remarkable since RIK-10+ is derived from the core antimicrobial region of LL-37 discovered based on structural studies (Li, et al. (2006) J. Am. Chem. Soc., 128(17):5776-5785). As these peptides are very small and highly selective, they are interesting candidates for further development. While high cationcity has been recognized to be important for killing Gram-negative bacteria, this study also revealed the importance of charge distribution patterns for such a peptide activity. LL-10+ with charged residues mostly at the C-terminus was weakest in inhibiting E. coli, while other peptides with higher activity placed their basic charges mainly at the N-terminus. When the sequences for LL-10+ and RIK-10+ were reversed, their activity in combating E. coli also reversed. Since numerous natural AMPs in the APD deployed more basic amino acids at the N-terminal region, the observation uncovered one useful strategy for designing new peptide antibiotics to fight drug-resistant Gram-negative pathogens.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An antimicrobial peptide comprising the sequence X₁RX₂X₃X₄X₅X₆X₇ (SEQ ID NO: 47), wherein X₁ is R or K, X₂ is W or I, X₃ is W or V, X₄ is R or Q, X₅ is W or R, X₆ is I or W, and X₇ is R, K, or L.

2. The antimicrobial peptide of claim 1, with the proviso that the peptide is not KRIVQRIK (SEQ ID NO: 6).

3. The antimicrobial peptide of claim 1, wherein the peptide comprises
(SEQ ID NO: 12)
KRIWQRIK,
(SEQ ID NO: 13)
KRIWQRWK,
(SEQ ID NO: 14)
KRWWQRWK,
(SEQ ID NO: 15)
KRWWQWWK,
(SEQ ID NO: 16),
RRWWRWWR
or
(SEQ ID NO: 17)
RRWWRWWL.

4. The antimicrobial peptide of claim 1, wherein the peptide comprises RRWWRWWR (SEQ ID NO: 16).

5. The antimicrobial peptide of claim 1, wherein the peptide comprises the sequence X₁RWWX₄X₅WX₇ (SEQ ID NO: 48), wherein X₁ is R or K, X₄ is R or Q, X₅ is W or R, and X₇ is R or K.

6. The antimicrobial peptide of claim 5, wherein X₅ is W.

7. The antimicrobial peptide of claim 5, wherein X₄ is R.

8. The antimicrobial peptide of claim 1, wherein the peptide has 12 or fewer amino acids.

9. The antimicrobial peptide of claim 1, wherein said peptide comprises at least one D-amino acid.

10. The antimicrobial peptide of claim 8, wherein all of the amino acids are D-amino acids.

11. The antimicrobial peptide of claim 1, wherein said peptide is amidated.

12. An antimicrobial peptide comprises:
a) the sequence LX₁GX₂FFRKX₃K (SEQ ID NO: 49), wherein X₁ is L or W; X₂ is D or R; and X₃ is S or W;
b) the sequence RX₁X₂KX₃X₄X₅GK (SEQ ID NO: 50), wherein X₁ is K or W; X₂ is S or W; X₃ is E or K; X₄ is K or W; and X₅ is I or W;
c) the sequence KRX₁X₂QX₃X₄K (SEQ ID NO: 51), wherein X₁ is I or W; X₂ is V or W; X₃ is R or W; and X₄ is I or W;
d) the sequence RX₁KX₂FLRNX₃V (SEQ ID NO: 52), wherein X₁ is I or W; X₂ is D or R; and X₃ is L or W; or
e) the reverse sequence of any of the above.

13. The antimicrobial peptide of claim 12, wherein
a) the peptide comprises the sequence LWGRFFRKWK (SEQ ID NO: 30);
b) the peptide comprises the sequence RWWKKWWGK (SEQ ID NO: 31);
c) the peptide comprises the sequence KRWWQWWK (SEQ ID NO: 32);
d) the peptide comprises the sequence RWKRFLRNWV (SEQ ID NO: 33); or
e) the peptide comprises the sequence KWKRFFRGWL (SEQ ID NO: 34).

14. A composition comprising at least one antimicrobial peptide of claim 1 and at least one pharmaceutically acceptable carrier, optionally further comprising at least one antibiotic.

15. A composition comprising at least one antimicrobial peptide of claim 12 and at least one pharmaceutically acceptable carrier, optionally further comprising at least one antibiotic.

16. A method for inhibiting or treating a microbial infection and/or biofilm in a subject in need thereof, said method comprising administering to said subject at least one antimicrobial peptide of claim 1.

17. A method for inhibiting or treating a microbial infection and/or biofilm in a subject in need thereof, said method comprising administering to said subject at least one antimicrobial peptide of claim 12.

18. A method of screening a compound for antibacterial activity, wherein said method comprises contacting the bacteria with the compound in a diluted media and measuring the cytotoxicity of the compound.

19. The method of claim 18, wherein the diluted media is Mueller-Hinton Broth (MHB) diluted to 10% to 15% with water.

20. A method for generating a peptide with antimicrobial activity against Gram-negative bacteria, said method comprising synthesizing a peptide with the following criteria:
a) the peptide is 8 to 12 amino acids in length;
b) at least 50% of the N-terminal four amino acids of the peptide are R or K;
c) the hydrophobicity of the peptide is at least 40%; and
d) the peptide has a net charge of at least +3.


SEQUENCE LISTING (52 sequences)

SEQ ID NO: 1 - protein, 37 aa, Homo sapiens
LLGDFFRKSKEKIGKEFKRIVQRIKDFLRNLVPRTES

SEQ ID NO: 2 - protein, 31 aa, synthetic construct
LLGDFFRKSKEKIGKEFKRIVQRIKDFLRNL

SEQ ID NO: 3 - protein, 24 aa, synthetic construct
SKEKIGKEFKRIVQRIKDFLRNLV

SEQ ID NO: 4 - protein, 16 aa, synthetic construct
FKRIVQRIKDFLRNLV

SEQ ID NO: 5 - protein, 12 aa, synthetic construct
KRIVQRIKDFLR

SEQ ID NO: 6 - protein, 8 aa, synthetic construct
KRIVQRIK

SEQ ID NO: 7 - protein, 10 aa, synthetic construct
LLGDFFRKSK

SEQ ID NO: 8 - protein, 10 aa, synthetic construct
KEKIGKEFKR

SEQ ID NO: 9 - protein, 10 aa, synthetic construct
LRNLVPRTES

SEQ ID NO: 10 - protein, 9 aa, synthetic construct
RKSKEKIGK

SEQ ID NO: 11 - protein, 10 aa, synthetic construct
RIKDFLRNLV

SEQ ID NO: 12 - protein, 8 aa, synthetic construct
KRIWQRIK

SEQ ID NO: 13 - protein, 8 aa, synthetic construct
KRIWQRWK

SEQ ID NO: 14 - protein, 8 aa, synthetic construct
KRWWQRWK

SEQ ID NO: 15 - protein, 8 aa, synthetic construct
KRWWQWWK

SEQ ID NO: 16 - protein, 8 aa, synthetic construct
RRWWRWWR

SEQ ID NO: 17 - protein, 8 aa, synthetic construct
RRWWRWWL

SEQ ID NO: 18 - protein, 34 aa, synthetic construct
LLGDFFRKSKEKIGKEFKRIVQRIKDFLRNLVPR

SEQ ID NO: 19 - protein, 29 aa, synthetic construct
GVFDIIKGAGKQLIARAMGKIAEKVGLNK

SEQ ID NO: 20 - protein, 34 aa, synthetic construct
ACDTATCVTHRLAGLLSRSGGVVKNNFVPTNVGS

SEQ ID NO: 21 - protein, 44 aa, synthetic construct
GIWSSIKNLASKAWNSDIGQSLRNKAAGAINKFVADKIGVTPSQ

SEQ ID NO: 22 - protein, 23 aa, synthetic construct
NGNLLGGLLRPVLGVVKGLTGGL

SEQ ID NO: 23 - protein, 32 aa, synthetic construct
RWKIFKKIEKVGQNIRDGIVKAGPAVAVVGQA

SEQ ID NO: 24 - protein, 28 aa, synthetic construct
SWLSKTAKKLENSAKKRIAEGIAIAIQG

SEQ ID NO: 25 - protein, 36 aa, synthetic construct
GWLKKIGKKIERVGQHTRDATIQGLGIAQQAANVAA

SEQ ID NO: 26 - protein, 39 aa, synthetic construct
KWKFGKKLERIGQNVFRAAEKVLPVATGYAQLPATLAGA

SEQ ID NO: 27 - protein, 34 aa, synthetic construct
RWKIFKRIEKVGRNVRDGVIKAGPAVAVLGQAKA

SEQ ID NO: 28 - protein, 32 aa, synthetic construct
RWKFGKKLERMGKRIFKATEKGLPVATGVAAL

SEQ ID NO: 29 - protein, 33 aa, synthetic construct
GGLKKLGKKLEGAGKRVFKASEKALPVVVGIKA

SEQ ID NO: 30 - protein, 10 aa, synthetic construct
LWGRFFRKWK

SEQ ID NO: 31 - protein, 9 aa, synthetic construct
RWWKKWWGK

SEQ ID NO: 32 - protein, 8 aa, synthetic construct
KRWWQWWK

SEQ ID NO: 33 - protein, 10 aa, synthetic construct
RWKRFLRNWV

SEQ ID NO: 34 - protein, 10 aa, synthetic construct
KWKRFFRGWL

SEQ ID NO: 35 - protein, 10 aa, synthetic construct
VWNRLFRKWR

SEQ ID NO: 36 - protein, 32 aa, Leptodactylus pustulatus
GVFDIIKGAGKQLIARAMGKIAEKVGLNKDGN

SEQ ID NO: 37 - protein, 37 aa, Homo sapiens
ACDTATCVTHRLAGLLSRSGGVVKNNFVPTNVGSKAF

SEQ ID NO: 38 - protein, 47 aa, vaejovis mexicanus
GIWSSIKNLASKAWNSDIGQSLRNKAAGAINKFVADKIGVTPSQAAS

SEQ ID NO: 39 - protein, 26 aa, Pleurodema thaul
NGNLLGGLLRPVLGVVKGLTGGLGKK

SEQ ID NO: 40 - protein, 35 aa, Bombyx mori
RWKIFKKIEKVGQNIRDGIVKAGPAVAVVGQAATI

SEQ ID NO: 41 - protein, 31 aa, Anisakis sp.
SWLSKTAKKLENSAKKRIAEGIAIAIQGGPR

SEQ ID NO: 42 - protein, 39 aa, Drosophila melanogaster
GWLKKIGKKIERVGQHTRDATIQGLGIAQQAANVAATAR

SEQ ID NO: 43 - protein, 42 aa, Panorpa sp.
KWKFGKKLERIGQNVFRAAEKVLPVATGYAQLPATLAGAKQG

SEQ ID NO: 44 - protein, 37 aa, Phyllodes eyndhovii
RWKIFKRIEKVGRNVRDGVIKAGPAVAVLGQAKALGK

SEQ ID NO: 45 - protein, 35 aa, Tunga penetrans
RWKFGKKLERMGKRIFKATEKGLPVATGVAALARG

SEQ ID NO: 46 - protein, 36 aa, Aedes aegypti
GGLKKLGKKLEGAGKRVFKASEKALPVVVGIKAIGK

SEQ ID NO: 47 - skipped

SEQ ID NO: 48 - protein, 8 aa, synthetic construct
  VARIANT 1: note = X is R or K
  VARIANT 5: note = X is R or Q
  VARIANT 6: note = X is W or R
  VARIANT 8: note = X is R or K
XRWWXXWX

SEQ ID NO: 49 - protein, 10 aa, synthetic construct
  VARIANT 2: note = X is L or W
  VARIANT 4: note = X is D or R
  VARIANT 9: note = X is S or W
LXGXFFRKXK

SEQ ID NO: 50 - protein, 9 aa, synthetic construct
  VARIANT 2: note = X is K or W
  VARIANT 3: note = X is S or W
  VARIANT 5: note = X is E or K
  VARIANT 6: note = X is K or W
  VARIANT 7: note = X is I or W
RXXKXXXGK

SEQ ID NO: 51 - protein, 8 aa, synthetic construct
  VARIANT 3: note = X is I or W
  VARIANT 4: note = X is V or W
  VARIANT 6: note = X is R or W
  VARIANT 7: note = X is I or W
KRXXQXXK

SEQ ID NO: 52 - protein, 10 aa, synthetic construct
RXKXFLRNXV